SEQUENCE LISTING (1 sequence)

SEQ ID NO: 1 - protein, 345 aa, synthetic construct
GRGEAETRECLYYNANWELERTNQSGVERCEGEKDKRLHCYASWRNSSGSLEIVKKGCWLDDFNCYDRDTCVATKENPQVYFCCCEGNMCNERFTHLPEAGGPEVTYEPPPTAPTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0188148 A1
(43) Pub. Date: Jun. 12, 2025

(54) METHODS OF ADMINISTERING AN ACTIVIN RECEPTOR TYPE IIB VARIANT

(71) Applicant: Keros Therapeutics, Inc., Lexington, MA (US)

(72) Inventors: Jasbir S. SEEHRA, Lexington, MA (US); Jennifer LACHEY, Lincoln, MA (US); Harveen Dhillon NATARAJAN, Braintree, MA (US); Christopher R. ROVALDI, Swampscott, MA (US)

(21) Appl. No.: 19/067,565

(22) Filed: Feb. 28, 2025

Related U.S. Application Data
(63) Continuation of application No. PCT/US2023/073749, filed on Sep. 8, 2023.
(60) Provisional application No. 63/530,761, filed on Aug. 4, 2023.
(60) Provisional application No. 63/405,260, filed on Sep. 9, 2022.

(51) Int. Cl. C07K 14/71; A61K 38/00

(52) U.S. Cl. CPC C07K 14/71; A61K 38/00; C07K 2319/30

(57) ABSTRACT

The invention features methods of treating a disease or condition associated with elevated activin signaling (e.g., a bone disease, pulmonary hypertension, fibrosis, a muscle disease, a metabolic disease, thrombocytopenia, or neutropenia) by administering to a human subject a polypeptide including an extracellular ActRIIB variant fused to an Fc domain in an amount of 1.5 mg/kg to 4.5 mg/kg at a frequency of once every 28 days.

### SEQUENCE LISTING

The instant application contains a Sequence Listing which has been submitted electronically in XML file format and is hereby incorporated by reference in its entirety. Said XML copy, created on Feb. 27, 2025, is named “51184-050004_Sequence_Listing_2_27_25” and is 2,119 bytes in size.

### BACKGROUND OF THE INVENTION

Duchenne muscular dystrophy (DMD), facioscapulohumeral muscular dystrophy (FSHD), inclusion body myositis (IBM), and amyotrophic lateral sclerosis (ALS) are examples of muscle diseases that involve weakness and atrophy of muscles and/or motor neurons that control voluntary muscle movements. DMD is caused by mutations in the X-linked dystrophin gene and characterized by progressive muscle degeneration and weakness in all skeletal muscles. FSHD particularly affects skeletal muscles of the face, shoulders, upper arms, and lower legs. IBM is an inflammatory muscle disease that mainly affects muscles of the thighs and muscles of the arms that control finger and wrist flexion. ALS is a motor neuron disease characterized by stiff muscles, muscle twitching, and muscle atrophy throughout the body due to the degeneration of the motor neurons. Efforts to improve treatment and survival of subjects having these devastating muscle diseases have not been successful.

Healthy bone undergoes a constant remodeling that involves both bone breakdown and bone growth. Bone growth is mediated by the osteoblast cell type whereas the osteoclasts resorb the bone. Pathology occurs when these systems fall out of balance either through downregulation of the anabolic program, upregulation of the catabolic system or a combination of both, resulting in a net bone loss. Therefore, controlling the balance in bone remodeling can be useful for promoting the healing of damage to bone as well as the treatment of disorders, such as osteoporosis, associated with loss of bone mass and bone demineralization.

Bone damage can result from a range of root causes, including age-or cancer-related bone loss, genetic conditions, or adverse side effects of drug treatment. The World Health Organization estimates that osteoporosis alone affects 75 million people in the U.S., Europe, and Japan, and is a significant risk factor in bone damage. In general, the whole of bone loss represents pathological states for which there are few effective treatments. Treatment instead focuses on immobilization, exercise, and dietary modifications rather than agents that directly promote bone growth and increase bone density. With respect to osteoporosis, estrogen, calcitonin, osteocalcin with vitamin K, or high doses of dietary calcium are all used as therapeutic interventions. Other therapeutic approaches to osteoporosis include bisphosphonates, parathyroid hormone, parathyroid hormone related protein, calcimimetics, statins, anabolic steroids, lanthanum and strontium salts, and sodium fluoride. Such therapeutics, however, are often associated with undesirable side effects.

Fibrosis is the formation of excess connective tissue in an organ or tissue. The connective tissue, which can form in response to damage (e.g., injury) or as part of an immune response (e.g., an inflammatory response), can disrupt the structure and function of the organ or tissue in which it forms, leading to an increase in tissue stiffness. Fibrosis can occur in many organs and tissues within the body, including the lung (e.g., pulmonary fibrosis, cystic fibrosis), liver (e.g., cirrhosis), heart (e.g., endomyocardial fibrosis or fibrosis after myocardial infarction), brain (e.g., glial scar formation), skin (e.g., formation of keloids), kidney (e.g., renal fibrosis), and eye (e.g., corneal fibrosis), among others; and is known to be associated with certain medical treatments (e.g., chemotherapy, radiation therapy, and surgery). There are limited treatment options for patients with fibrosis, and most treatments are focused on improving quality of life or temporarily slowing disease progression.

Thrombocytopenia is a condition characterized by abnormally low levels of platelets, also called thrombocytes, in the blood, and occurs when the bone marrow makes too few platelets or when too many platelets are destroyed or accumulate within an enlarged spleen. Patients with thrombocytopenia may experience internal or external bleeding, bleeding under the skin, and/or bruising. Treatment for thrombocytopenia depends on its cause and severity and is primarily focused on preventing death or disability caused by bleeding. Certain types of thrombocytopenia (e.g., immune thrombocytopenia) may be treated using corticosteroids, but other types of thrombocytopenia may require splenectomy or platelet transfusion.

Neutropenia is a condition characterized by an abnormally low number of neutrophils in the blood. Neutrophils typically constitute 45% to 75% of all white blood cells in the bloodstream and serve as the primary defense against infections. Reduced numbers of neutrophils can lead to difficulty in controlling infections and increase the risk of dying from an infection. In patients with severe neutropenia, infections can rapidly become severe or fatal. Antibiotics are used treat infection in patients having neutropenia, but treatments for neutropenia itself are limited, and primarily involve the use of growth factors, such as colony stimulating factors, to stimulate the production of white blood cells. Blood transfusions have not proven effective.

Pulmonary hypertension (PH) is a serious condition characterized by higher than normal pressure in the blood vessels between the lungs and the heart. PH can be categorized into five major types: arterial (PAH), venous (PH secondary to left-sided heart disease), hypoxic (PH caused by lung disease), thromboembolic (PH caused by chronic arterial obstruction, e.g., blood clots), or miscellaneous (PH with unclear or multifactorial mechanisms), also known as WHO groups I-V. PAH features increased pressure in blood vessels of the lungs caused by obstruction in or narrowing of small blood vessels in the lungs due to scarring. This leads to increased resistance to blood flow through the lungs and forces the right side of the heart to work harder, which may lead to heart failure, reduced blood oxygenation, and reduced life expectancy. PAH can be idiopathic (e.g., having no identifiable cause), heritable (e.g., familial, often due to a genetic mutation), or may be related to drug use (e.g., methamphetamine or cocaine use), infection (e.g., HIV infection or schistosomiasis), cirrhosis of the liver, congenital heart abnormalities, or connective tissue/autoimmune disorders (e.g., scleroderma or lupus). Treatments for PH include vasodilators, anticoagulants, and supplemental oxygen, but these treatments manage disease symptoms rather than targeting the biological mechanisms that cause the disease.

Excess body weight is an increasing problem in large parts of the world, with about 39% of adults aged 18 years and over found to be overweight in 2016 and about 13% of the world's adult population found to be obese. Increased visceral and subcutaneous fact causes dysfunction of various organs. Excessive body weight is a risk factor for an array of complications, including diabetes (e.g., Type 1 and Type 2 diabetes), cardiovascular disease, and several forms of cancer. Insulin resistance is also associated with obesity and results in pancreatic tissues producing an elevated amount of insulin. Once pancreatic B cells can no longer produce sufficient insulin to meet the demand, hyperglycemia occurs and Type 2 diabetes develops. Adipocytes, which are increased in obesity, are believed to play a role in this process. Despite the prevalence of obesity and metabolic diseases few therapeutic options are available. There exists a need for effective treatments for muscular diseases, bone diseases, thrombocytopenia, neutropenia, fibrosis, PH, and metabolic diseases.

### SUMMARY OF THE INVENTION

Described herein are methods of treating a subject having or at risk of developing a bone disease, pulmonary hypertension, fibrosis, a muscle disease, a metabolic disease, thrombocytopenia, neutropenia, or a disease or condition that can be treated with erythropoietin or an erythropoiesis-stimulating agent by administering to the subject a polypeptide including an extracellular activin receptor type IIB (ActRIIB) variant fused to an Fc domain. The polypeptide can be administered to a human subject once every 28 days at a dose of 1.5 mg/kg to 4.5 mg/kg to treat a disease or condition that would benefit from reducing or inhibiting endogenous activin signaling (e.g., a disease or condition in which an activin receptor ligand, such as activin A, activin B, myostatin, or GDF-11 is elevated compared to levels observed in a healthy subject).

Exemplary embodiments of the invention are described in the enumerated paragraphs below.
E1. A method of treating a human subject having or at risk of developing a bone disease or pulmonary hypertension (PH), the method comprising the step of administering to the subject a polypeptide of SEQ ID NO: 1 in an amount of 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) at a frequency of once every 28 days.
E2. The method of E1, wherein the subject has or is at risk of developing a bone disease.
E3. The method of E1 or E2, wherein the bone disease is osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss.
E4. The method of E3, wherein the bone disease is osteoporosis.
E5. The method of E3 or E4, wherein the osteoporosis is primary osteoporosis.
E6. The method of E5, wherein the primary osteoporosis is age-related osteoporosis or hormone-related osteoporosis.
E7. The method of E3 or E4, wherein the osteoporosis is secondary osteoporosis.
E8. The method of E7, wherein the secondary osteoporosis is immobilization-induced osteoporosis or glucocorticoid-induced osteoporosis, or results from an endocrinopathy, a gastrointestinal disorder, a hematological disorder, an autoimmune disorder, renal disease, a medication, alcoholism, or transplantation.
E9. The method of E3, wherein the bone disease is osteogenesis imperfecta.
E10. The method of E3, wherein the bone disease is osteopenia.
E11. The method of E3, wherein the bone disease is bone fracture.
E12. The method of E3, wherein the bone disease is bone cancer or cancer metastasis-related bone loss.
E13. The method of E3 or E12, wherein the cancer is multiple myeloma.
E14. The method of E3, wherein the bone disease is Paget's disease.
E15. The method of E3, wherein the bone disease is renal osteodystrophy.
E16. The method of E3, wherein the bone disease is treatment-related bone loss.
E17. The method of E3 or E16, wherein the treatment is FGF-21 treatment, GLP-1 treatment, treatment with an FGF-21-or GLP-1-containing therapeutic, cancer therapy (e.g., chemotherapy or radiation), bariatric surgery (e.g., gastric bypass), androgen or estrogen deprivation therapy, or treatment for obesity or Type 2 diabetes.
E18. The method of E3, wherein the bone disease is diet-related bone loss.
E19. The method of E3 or E18, wherein the diet-related bone loss is rickets.
E20. The method of E3, wherein the bone disease is low gravity-related bone loss.
E21. The method of E3, wherein the bone disease is immobility-related bone loss.
E22. The method of E3, wherein the bone disease is neuromuscular disease-related bone loss.
E23. The method of E3, wherein the bone disease is burn-induced bone loss.
E24. The method of E3, wherein the bone disease is anorexia-related bone loss.
E25. The method of any one of E1-E24, wherein the subject is at risk of bone fracture.
E26. The method of any one of E1-E25, wherein the method increases bone formation in the subject or increases the rate of bone formation.
E27. The method of any one of E1-E26, wherein the method decreases bone resorption in the subject or reduces the rate of bone resorption.
E28. The method of any one of E1-E27, wherein the method decreases bone loss in the subject.
E29. The method of any one of E1-E28, wherein the method increases osteoblast activity or osteoblastogenesis.
E30. The method of any one of E1-E29, wherein the method decreases osteoclast activity or decreases osteoclastogenesis.
E31. The method of any one of E1-E30, wherein the method decreases the risk or occurrence of bone fracture.
E32. The method of any one of E1-E31, wherein the method increases bone strength.
E33. The method of any one of E1-E32, wherein the method increases bone mineral density.
E34. The method of any one of E1-E33, wherein the bone is cortical bone.
E35. The method of any one of E1-E33, wherein the bone is trabecular bone.
E36. The method of E1, wherein the subject has or is at risk of developing PH.
E37. The method of E1 or E36, wherein the PH is pulmonary arterial hypertension (PAH), venous PH, hypoxic PH, thromboembolic PH, or miscellaneous PH.
E38. The method of E37, wherein the PH is pulmonary arterial hypertension (PAH) (World Health Organization (WHO) Group 1 PH).
E39. The method of E38, wherein the PAH is idiopathic PAH.
E40. The method of E38, wherein the PAH is heritable PAH.
E41. The method of E38, wherein the PAH is associated with HIV infection, schistosomiasis, cirrhosis of the liver, a congenital heart abnormality, portal hypertension, pulmonary veno-occlusive disease, pulmonary capillary hemangiomatosis, a connective tissue disorder, an autoimmune disorder (e.g., scleroderma, lupus (systemic lupus erythematosus), mixed connective tissue disease, Sjogren syndrome, an inflammatory idiopathic myopathy, or rheumatoid arthritis), drug use or abuse (e.g., use of cocaine or methamphetamine), a toxin, or congenital systemic-pulmonary intracardiac shunt.
E42. The method of any one of E38-E41, wherein the subject has WHO/New York Heart Association (NYHA) Functional Class (FC) Il symptoms or FC III symptoms.
E43. The method of any one of E38-E42, wherein the subject has a 6-minute walk distance ≥150 and ≤500 meters.
E44. The method of any one of E38-E43, wherein the method further comprises administering a PAH background therapy.
E45. The method of any one of E38-E43, wherein the human subject is on a PAH background therapy.
E46. The method of E44 or E45, wherein the PAH background therapy is an endothelin-receptor antagonist (ERA) (e.g., ambrisentan, bosentan, macitentan, or thelin), a phosphodiesterase-5 inhibitor (PDE5-I) (e.g., e.g., sildenafil, tadalafil, or vardenafil), a soluble guanylate cyclase (sGC) stimulator (e.g., riociguat or cinaciguat), or a prostacyclin analogue or receptor agonist (e.g., epoprostenol, iloprost, treprostinil, beraprost, or selexipag), an anticoagulant (e.g., warfarin), a diuretic, oxygen therapy, digoxin, a calcium channel blocker (e.g., nifedipine, diltiazem, or amlodipine), atrial septostomy, pulmonary thromboendarterectomy, an ASK-1 inhibitor (e.g., CIIA, SCH79797, GS-4997, MSC2032964A, a 3H-naphtho [1,2,3-de]quiniline-2,7-diones, NQDI-1, 2-thioxo-thiazolidines, or 5-bromo-3-(4-oxo-2-thioxo-thiazolidine-5-ylidene)-1,3-dihydro-indol-2-one), a NF-κB antagonist (e.g., dh404, CDDO-epoxide, 2.2-difluoropropionamide, C28 imidazole (CDDO-Im), 2-cyano-3,12-dioxoolean-1,9-dien-28-oic acid (CDDO), 3-Acetyloleanolic Acid, 3-Triflouroacetyloleanolic Acid, 28-Methyl-3-acetyloleanane, 28-Methyl-3-trifluoroacetyloleanane, 28-Methyloxyoleanolic Acid, SZC014, SCZ015, SZC017, PEGylated derivatives of oleanolic acid, 3-O-(beta-D-glucopyranosyl) oleanolic acid, 3-O-[beta-D-glucopyranosyl-(1→3)-beta-D-glucopyranosyl] oleanolic acid, 3-O-[beta-D-glucopyranosyl-(1→2)-beta-D-glucopyranosyl] oleanolic acid, 3-O-[beta-D-glucopyranosyl-(1→3)-beta-D-glucopyranosyl]oleanolic acid 28-O-beta-D-glucopyranosyl ester, 3-O-[beta-D-glucopyranosyl-(1→2)-beta-D-glucopyranosyl] oleanolic acid 28-O-beta-D-glucopyranosyl ester, 3-O-[a-L-rhamnopyranosyl-(1→3)-beta-D-glucuronopyranosyl] oleanolic acid, 3-O-[alpha-L-rhamnopyranosyl-(1→3)-beta-D-glucuronopyranosyl] oleanolic acid 28-O-beta-D-glucopyranosyl ester, 28-O-β-D-glucopyranosyl-oleanolic acid, 3-O-β-D-glucopyranosyl (1→3)-3-D-glucopyranosiduronic acid (CS1), oleanolic acid 3-O-β-D-glucopyranosyl (1→3)-3-D-glucopyranosiduronic acid (CS2), methyl 3,11-dioxoolean-12-en-28-olate (DIOXOL), ZCVI₄-2, or Benzyl 3-dehydr-oxy-1,2,5-oxadiazolo [3′,4′: 2,3]oleanolate), or lung and/or heart transplantation.
E47. The method of E46, wherein the PAH background therapy is an ERA, a PDE5-1, an sGC stimulator, or a prostacyclin analogue or receptor agonist.
E48. The method of any one of E44-E47, wherein the subject is administered a single PAH background therapy.
E49. The method of any one of E44-E47, wherein the subject is administered two or more (e.g., 2, 3, 4, 5, or more) PAH background therapies (e.g., an ERA and a PDE5-I, an ERA and an sGC stimulator, an ERA and a prostacyclin receptor analogue or agonist, a PDE5-I and a prostacyclin receptor analogue or agonist, an sGC stimulator, and a prostacyclin receptor analogue or agonist, an ERA, a PDE5-I, and a prostacyclin receptor analogue or agonist, or an ERA, an sGC stimulator, and a prostacyclin receptor analogue or agonist).
E50. The method of any one of E44-E49, wherein the PAH background therapy is administered for at least 90 days prior to treatment initiation with the polypeptide of SEQ ID NO: 1 (e.g., stable background therapy for at least 90 days prior to treatment initiation, defined as no change in dose/regimen of PAH background therapy for at least 90 days prior to treatment initiation).
E51. The method of E37, wherein the PH is venous PH (WHO Group 2 PH).
E52. The method of E51, wherein the venous PH is associated with left ventricular systolic dysfunction, left ventricular diastolic dysfunction, valvular heart disease, congenital cardiomyopathy, or congenital or acquired pulmonary venous stenosis.
E53. The method of E37, wherein the PH is hypoxic PH (WHO Group 3 PH).
E54. The method of E53, wherein the hypoxic PH is associated with chronic obstructive pulmonary disease (e.g., emphysema), interstitial lung disease, sleep-disordered breathing (e.g., sleep apnea), a lung disease (e.g., pulmonary fibrosis), an alveolar hypoventilation disorder, chronic exposure to high altitude, or a developmental abnormality.
E55. The method of E37, wherein the PH is thromboembolic PH (WHO Group 4 PH).
E56. The method of E55, wherein the thromboembolic PH is associated with chronic thromboembolic pulmonary hypertension, pulmonary emboli, angiosarcoma, arteritis, congenital pulmonary artery stenosis, or parasitic infection.
E57. The method of E37, wherein the PH is miscellaneous PH (WHO Group 5 PH).
E58. The method of E57, wherein the miscellaneous PH is associated with a hematologic disease (e.g., chronic hemolytic anemia, sickle cell disease), a systemic disease (e.g., sarcoidosis, pulmonary Langerhans cell histiocytosis, lymphangioleiomyomatosis, neurofibromatosis, or vasculitis), a metabolic disorder (e.g., glycogen storage disease, Gaucher disease, or thyroid diseases), pulmonary tumoral thrombotic microangiopathy, fibrosing mediastinitis, chronic kidney failure, or segmental pulmonary hypertension.
E59. The method of any one of E36-E58, wherein the method reduces the frequency or severity of one or more symptoms of PH (e.g., reduces the severity or frequency of one or more of shortness of breath (dyspnea), fatigue, swelling (e.g., edema) of the legs, feet, belly (ascites), or neck, chest pain or pressure, racing pulse or heart palpitations, bluish color to lips or skin (cyanosis), dizziness, or fainting), reduces inflammation, or reduces fibrosis.
E60. The method of any one of E36-E59, wherein the method prevents PH (e.g., prevents the development of PH).
E61. The method of any one of E36-E60, wherein the method slows or inhibits the progression of PH.
E62. The method of any one of E36-E61, wherein the method reduces the risk of developing PH.
E63. The method of any one of E36-E62, wherein the method reduces pulmonary vascular remodeling.
E64. The method of any one of E36-E63, wherein the method reduces vascular remodeling in the heart.
E65. The method of any one of E36-E64, wherein the method reduces right ventricular hypertrophy and/or right ventricle overload.
E66. The method of any one of E36-E65, wherein the method reduces pulmonary vascular resistance (e.g., reduces pulmonary vascular resistance compared to measurements taken prior to treatment, for example, by 24 weeks of treatment with the polypeptide of SEQ ID NO: 1).
E67. The method of any one of E36-E66, wherein the method improves cardiac output and/or improves performance in the 6-minute walk test (e.g., improves 6-minute walk distance, e.g., improves performance compared to measurements taken prior to treatment, for example, by 24 weeks of treatment with the polypeptide of SEQ ID NO: 1).
E68. The method of any one of E36-E67, wherein the method reduces bone loss.
E69. The method of any one of E36-E68, wherein the method reduces pulmonary arterial muscularization or pulmonary arterial wall thickening.
E70. The method of any one of E36-E69, wherein the method reduces right ventricular compensation.
E71. The method of any one of E36-E70, wherein the method delays the development of PH.
E72. The method of any one of E36-E71, wherein the method improves WHO/NYHA FC (e.g., compared to baseline pre-treatment assessments).
E73. The method of any one of E36-E72, wherein the method improves one or more of mean pulmonary arterial pressure (mPAP), cardiac output (CO), cardiac index (CI), pulmonary artery wedge pressure (PAWP), right atrial pressure (RAP), mixed venous oxygen saturation (SvO₂), stroke volume (SV), stroke volume index (SVI), or pulmonary artery compliance (PAC) (e.g., compared to baseline pre-treatment measurements, for example, by 24 or 96 weeks of treatment with the polypeptide of SEQ ID NO: 1).
E74. The method of any one of E36-E73, wherein the method attenuates clinical worsening (e.g., reduces the incidence of clinical worsening or increases the time to clinical worsening, e.g., the incidence or time to first clinical worsening).
E75. The method of any one of E36-E74, wherein the method improves risk stratification measures (e.g., leads to an improvement or a maintenance of low risk in ESC/ERC 4-strata risk category assessment or leads to an improvement in REVEAL (Registry to Evaluate Early and Long-term PAH Disease Management) Lite 2 and/or COMPERA 2.0 as compared to baseline pre-treatment measurements).
E76. The method of any one of E36-E75, wherein the method improves physical activity (overall activity) (e.g., compared to baseline pre-treatment measurements, e.g., as measured by actigraphy).
E77. The method of any one of E36-E76, wherein the method improves health-related quality of life (HRQOL) (e.g., assessed using Pulmonary Arterial Hypertension-Symptoms and Impact (PAH-SYMPACT) and/or emPHasis-10 compared to baseline pre-treatment assessments).
E78. The method of any one of E36-E77, wherein the method reduces NT-proBNP (e.g., compared to baseline pre-treatment levels).
E79. A method of treating a human subject having or at risk of developing fibrosis, a disease or condition involving muscle weakness or atrophy (i.e., a muscle disease), a metabolic disease, thrombocytopenia, neutropenia, or a disease or condition that can be treated with erythropoietin or an erythropoiesis-stimulating agent, the method comprising the step of administering to the subject a polypeptide of SEQ ID NO: 1 in an amount of 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) once every 28 days.
E80. The method of E79, wherein the subject has or is at risk of developing fibrosis.
E81. The method of E79 or E80, wherein the fibrosis is chemotherapeutic drug-induced fibrosis, radiation-induced fibrosis, pulmonary fibrosis, hepatic fibrosis, renal fibrosis (e.g., fibrosis related to chronic kidney disease), corneal fibrosis, heart fibrosis, bone marrow fibrosis, myelofibrosis, mediastinal fibrosis, retroperitoneal fibrosis, arthrofibrosis, osteoarticular fibrosis, tissue fibrosis, a tumor stroma, a desmoplastic tumor, a surgical adhesion, a hypertrophic scar, or a keloid.
E82. The method of E81, wherein the tissue fibrosis is fibrosis affecting a tissue selected from the group consisting of muscle tissue, skin epidermis, skin dermis, tendon, cartilage, pancreatic tissue, uterine tissue, neural tissue, testis, ovary, adrenal gland, artery, vein, bone marrow, colon, small intestine, large intestine, biliary tract, and gut.
E83. The method of any one of E79-E81, wherein the fibrosis is fibrosis associated with a wound, a burn, hepatitis B or C infection, fatty liver disease, Schistosoma infection, kidney disease (e.g., chronic kidney disease), heart disease, macular degeneration, Crohn's disease, retinal or vitreal retinopathy, systemic or local scleroderma, atherosclerosis, or restenosis.
E84. The method of any one of E80-E83, wherein the method improves the function of a fibrotic tissue or organ.
E85. The method of any one of E80-E84, wherein the method slows, inhibits, or reverses the progression of fibrosis.
E86. The method of any one of E80-E85, wherein the method reduces the risk of developing fibrosis.
E87. The method of any one of E80-E86, wherein the method delays or prevents the development of fibrosis.
E88. The method of any one of E80-E87, wherein the method reduces fibrosis or reduces (e.g., reduces the frequency or severity of) one or more symptom of fibrosis.
E89. The method of E79, wherein the subject has or is at risk of developing a disease or condition involving muscle weakness or atrophy (i.e., a muscle disease).
E90. The method of E79 or E89, wherein the disease or condition involving muscle weakness or atrophy (the muscle disease) is a neuromuscular disease, sarcopenia, cachexia, disuse atrophy, treatment-related muscle loss or atrophy, hypotonia, muscle loss or atrophy associated with hypoxia, or muscle loss or atrophy associated with a burn injury.
E91. The method of E90, wherein the disease or condition is a neuromuscular disease.
E92. The method of any one of E3, E22, E90, and E91, wherein the neuromuscular disease is a muscular dystrophy, amyotrophic lateral sclerosis (ALS), autonomic neuropathy, botulism, Charcot-Marie-Tooth disease (CMT), chronic inflammatory demyelinating polyradiculoneuropathy, congenital myasthenic syndrome, a congenital myopathy, cramp-fasciculation syndrome, dermatomyositis, diabetic neuropathy, a distal myopathy, a dystrophinopathy, an endocrine myopathy, a focal muscular atrophy, glycogen storage disease type II, Guillain-Barre syndrome, hereditary spastic paraplegia, inclusion body myositis (IBM), Isaac's syndrome, Kearns-Sayre syndrome, Kennedy disease, Lambert-Eaton myasthenic syndrome, a metabolic myopathy, a metabolic neuropathy, a mitochondrial myopathy, a motor neuron disease, multiple sclerosis, myasthenia gravis, myotonic dystrophy, a necrotizing myopathy, neuromyotonia, neuropathy of Friedreich's Ataxia, a nutritional neuropathy, peripheral neuropathy, polymyositis, primary lateral sclerosis, Schwartz-Jampel Syndrome, small fiber neuropathy, spinal and bulbar muscular atrophy, spinal muscular atrophy (SMA), spinal muscular atrophy with respiratory distress type 1, stiff person syndrome, toxic neuropathy, or Troyer syndrome.
E93. The method of E92, wherein the neuromuscular disease is a muscular dystrophy.
E94. The method of E93, wherein the muscular dystrophy is Duchenne muscular dystrophy (DMD), facioscapulohumeral muscular dystrophy (FSHD), Becker muscular dystrophy (BMD), myotonic dystrophy (DM), congenital muscular dystrophy, limb-girdle muscular dystrophy (LGMD), distal muscular dystrophy (DD), oculopharyngeal muscular dystrophy (OPMD), or Emery-Dreifuss muscular dystrophy (EDMD).
E95. The method of E94, wherein the muscular dystrophy is DMD.
E96. The method of E94, wherein the muscular dystrophy is FSHD.
E97. The method of E94, wherein the muscular dystrophy is BMD.
E98. The method of E94, wherein the muscular dystrophy is DM.
E99. The method of E94, wherein the muscular dystrophy is LGMD.
E100. The method of E94, wherein the muscular dystrophy is DD.
E101. The method of E94, wherein the muscular dystrophy is OPMD.
E102. The method of E94, wherein the muscular dystrophy is EDMD.
E103. The method of E94, wherein the muscular dystrophy is a congenital muscular dystrophy.
E104. The method of E103, wherein the congenital muscular dystrophy is congenital muscular dystrophy type 1A (MDC1A), congenital muscular dystrophy type 1C (MDC1C), congenital muscular dystrophy type 1D (MDC1D), congenital muscular dystrophy type 1B (MDC1B),

Fukuyama congenital muscular dystrophy (FCMD), muscle-eye-brain disease (MEB), Walker-Warburg Syndrome (WWS), rigid spine muscular dystrophy (RSMD1), Ullrich congenital muscular dystrophy (UCMD), or muscular dystrophy associated with a mutation in integrin alpha 7, integrin alpha 9, docking protein 7, laminin A/C, SECIS binding protein 2, or choline kinase beta.
E105. The method of E104, wherein the congenital muscular dystrophy is MDC1A.
E106. The method of E104, wherein the congenital muscular dystrophy is MDC1B.
E107. The method of E104, wherein the congenital muscular dystrophy is MDC1C.
E108. The method of E104, wherein the congenital muscular dystrophy is MDC1D.
E109. The method of E104, wherein the congenital muscular dystrophy is FCMD.
E110. The method of E104, wherein the congenital muscular dystrophy is MEB.
E111. The method of E104, wherein the congenital muscular dystrophy is WWS.
E112. The method of E104, wherein the congenital muscular dystrophy is RSMD1.
E113. The method of E104, wherein the congenital muscular dystrophy is UCMD.
E114. The method of E92, wherein the neuromuscular disease is CMT.
E115. The method of E92, wherein the neuromuscular disease is ALS.
E116. The method of E92, wherein the neuromuscular disease is SMA.
E117. The method of E92, wherein the neuromuscular disease is IBM.
E118. The method of E92, wherein the neuromuscular disease is myasthenia gravis.
E119. The method of E92, wherein the neuromuscular disease is multiple sclerosis.
E120. The method of E90, wherein the disease or condition is sarcopenia.
E121. The method of E90, wherein the disease or condition is disuse atrophy.
E122. The method of E90, wherein the disease or condition is treatment-related muscle loss or atrophy.
E123. The method of E90 or E122, wherein the treatment is glucocorticoid treatment, FGF-21 treatment, GLP-1 treatment, treatment with an FGF-21-or GLP-1-containing therapeutic, bariatric surgery (e.g., gastric bypass), cancer therapy (e.g., chemotherapy or radiation), or treatment for obesity or Type 2 diabetes.
E124. The method of E90, wherein the disease or condition is hypotonia.
E125. The method of E90, wherein the disease or condition is muscle loss or atrophy associated with hypoxia.
E126. The method of E90, wherein the disease or condition is muscle loss or atrophy associated with a burn injury.
E127. The method of E90, wherein the disease or condition is cachexia.
E128. The method of E90 or E127, wherein the cachexia is cancer cachexia, HIV-related cachexia, cardiac cachexia (e.g., cachexia associated with heart failure), cachexia associated with chronic kidney disease, or pulmonary cachexia (e.g., cachexia associated with COPD).
E129. The method of any one of E89-E128, wherein the method increases muscle mass.
E130. The method of any one of E89-E129, wherein the method increases lean mass.
E131. The method of any one of E89-E130, wherein the method increases muscle strength.
E132. The method of E79, wherein the subject has or is at risk of developing a metabolic disease.
E133. The method of E79 or E132, wherein the metabolic disease is age-related metabolic disease.
E134. The method of E79 or E132, wherein the metabolic disease is treatment-related metabolic disease.
E135. The method of E134, wherein the treatment is treatment with a glucocorticoid (e.g., a corticosteroid, such as prednisone), a selective serotonin reuptake inhibitors (SSRI, e.g., paroxetine, mirtazapine, fluoxetine, escitalopram, or sertraline), a serotonin-norepinephrine reuptake inhibitors (SNRI), a tricyclic antidepressant (e.g., amitriptyline), a mood stabilizer (e.g., valproic acid or lithium), an antipsychotic (e.g., olanzapine, chlorpromazine, or clozapine), or a diabetes medication (e.g., insulin, chlorpropamide).
E136. The method of any one of E132-E135, wherein the metabolic disease is obesity, Type 1 diabetes, or Type 2 diabetes.
E137. The method of E136, wherein the metabolic disease is obesity.
E138. The method of E136, wherein the metabolic disease is Type 1 diabetes.
E139. The method of E136, wherein the metabolic disease is Type 2 diabetes.
E140. The method of any one of E132-E139, wherein the method reduces body weight and/or percentage of body weight gain of said subject.
E141. The method of any one of E132-E140, wherein the method reduces amount of body fat and/or percentage of body fat of said subject.
E142. The method of any one of E132-E141, wherein the method does not affect the appetite for food intake of said subject.
E143. The method of any one of E132-E142, wherein the method reduces adiposity of said subject.
E144. The method of any one of E132-E143, wherein the method reduces the weights of epididymal and perirenal fat pads of said subject.
E145. The method of any one of E132-E144, wherein, the method reduces the amount of subcutaneous, visceral, and/or hepatic fat of said subject.
E146. The method of any one of E132-E145, wherein the method lowers the level of fasting insulin of said subject.
E147. The method of any one of E132-E146, wherein the method lowers the level of blood glucose of said subject.
E148. The method of any one of E132-E147, wherein the method increases insulin sensitivity of said subject.
E149. The method of any one of E132-E148, wherein the method increases the rate of glucose clearance of said subject.
E150. The method of any one of E132-E149, wherein the method improves the serum lipid profile of said subject.
E151. The method of any one of E132-E150, wherein the method delays, reduces, or eliminates the need for insulin treatment.
E152. The method of any one of E132-E151, wherein the method reduces LDL.
E153. The method of any one of E132-E152, wherein the method reduces triglycerides.
E154. The method of any one of E132-E153, wherein the method regulates insulin biosynthesis and/or secretion from B-cells,
E155. The method of any one of E132-E154, wherein the method does not reduce lean mass.
E156. The method of E79, wherein the subject has or is at risk of developing thrombocytopenia.
E157. The method of E79 or E156, wherein the thrombocytopenia is associated with a bone marrow defect, a myelodysplastic syndrome, bone marrow transplantation, myelofibrosis, myelofibrosis treatment (e.g., treatment with a JAK inhibitor, such as with ruxolitinib, fedratinib, or pacritinib), ineffective hematopoiesis, Gaucher disease, aplastic anemia, Fanconi anemia, Diamond Blackfan anemia, Shwachman Diamond syndrome, heavy alcohol consumption, cirrhosis of the liver, cancer (e.g., leukemia or lymphoma), an autoimmune disease, a viral infection, a bacterial infection, an enlarged spleen, a vitamin deficiency, cancer treatment, thrombotic thrombocytopenia purpura, idiopathic thrombocytopeniarpura, disseminated intravascular coagulation, hemolytic uremic syndrome, paroxysmal nocturnal hemoglobinuria, a reduction of platelets caused by medication (medication-induced thrombocytopenia, e.g., thrombocytopenia caused by treatment with heparin, quinine, a sulfa-containing antibiotic, such as vancomycin, rifampin, or trimethoprim, or an anticonvulsant, such as phenytoin), hematopoietic stem cell transplantation, acquired amegakaryocytic thrombocytopenia, Pearson syndrome, dyskeratosis congenita, contraindication to transfusion, or a dilution of platelets caused by a blood transfusion.
E158. The method of E79 or E156, wherein the thrombocytopenia is familial thrombocytopenia.
E159. The method of E158, wherein the familial thrombocytopenia is May-Hegglin anomaly, Sebastian syndrome, Fechtner syndrome, Epstein's syndrome, Wiskott-Aldrich syndrome, congenital amegakaryocytic thrombocytopenia, platelet storage pool deficiency, Hermansky-Pudlak syndrome, Bernard-Soulier syndrome, Von Willebrand Disease Type 2B, ANKRD26-related thrombocytopenia, thrombocytopenia absent radius syndrome, familial platelet disorder with associated myeloid malignancy (FPD/AML), thrombocytopenia associated with a mutation in Filamin-A, or thrombocytopenia associated with a mutation in GATA-1.
E160. The method of E79 or E156, wherein the thrombocytopenia is immune thrombocytopenia.
E161. The method of any one of E156-E160, wherein the method increases platelet count (e.g., platelet levels), platelet production and/or megakaryocyte differentiation and/or maturation.
E162. The method of any one of E156-E161, wherein the method reduces the accumulation of platelet progenitor cells.
E163. The method of any one of E156-E162, wherein the method improves blood clotting, reduces bleeding events (e.g., reduces the incidence of bleeding events), and/or reduces bleeding in the skin of the subject.
E164. The method of any one of E156-E163, wherein the subject is identified as having thrombocytopenia prior to administration of the polypeptide of SEQ ID NO: 1.
E165. The method of any one of E156-E164, wherein the method further comprises identifying the subject as having thrombocytopenia prior to administration of the polypeptide of SEQ ID NO: 1.
E166. The method of any one of E156-E165, wherein the method further comprises evaluating platelet levels after administration of the polypeptide of SEQ ID NO: 1.
E167. The method of E79, wherein the subject has or is at risk of developing neutropenia.
E168. The method of E79 or E167, wherein the neutropenia is associated with a bone marrow defect, a myelodysplastic syndrome, bone marrow transplantation, myelofibrosis, ineffective hematopoiesis, aplastic anemia, Fanconi anemia, Diamond Blackfan anemia, Shwachman Diamond syndrome, paroxysmal nocturnal hemoglobinuria, Pearson syndrome, dyskeratosis congenita, cancer (e.g., leukemia), a vitamin deficiency, an enlarged spleen, an autoimmune disease, a viral infection, a bacterial infection, cancer treatment, a reduction in neutrophils caused by medication (e.g., medication used to treat overactive thyroid, such as methimazole and propylthiouracil; an antibiotic, such as vancomycin, penicillin G, trimethoprim, and oxacillin; an antiviral drug, such as ganciclovir and valganciclovir; an anti-inflammatory medication for ulcerative colitis or rheumatoid arthritis, such as sulfasalazine; a drug used to treat irregular heart rhythms, such as quinidine and procainamide; an anticonvulsant, such as phenytoin and valproate; an antipsychotic, such as clozapine; or levamisole), inflammation, hematopoietic stem cell transplantation, or contraindication to transfusion.
E169. The method of E79 or E167, wherein the neutropenia is chronic idiopathic neutropenia.
E170. The method of E79 or E167, wherein the neutropenia is familial neutropenia.
E171. The method of E79 or E167, wherein the familial neutropenia is cyclic neutropenia, chronic benign neutropenia, or severe congenital neutropenia (e.g., neutropenia associated with mutations in the genes ELANE (associated with SCN1), HAX1 (associated with SCN3), G6PC3 (associated with SCN4), GFI1 (associated with SCN2), CSF3R, WAS (associated with X-linked neutropenia/X-linked SCN), CXCR4, VPS45A (associated with SCN5), or JAGN1).
E172. The method of any one of E167-E171, wherein the method increases neutrophil count (e.g., neutrophil levels), neutrophil production, and/or the differentiation and/or maturation of progenitor cells (e.g., myeloid progenitors, myeloblasts, and/or myelocytes) into neutrophils.
E173. The method of any one of E167-E172, wherein the method reduces the subject's susceptibility to infection.
E174. The method of any one of E167-E173, wherein the subject is identified as having neutropenia prior to administration of the polypeptide of SEQ ID NO: 1.
E175. The method of any one of E167-E174, wherein the method further comprises identifying the subject as having neutropenia prior to administration of the polypeptide of SEQ ID NO: 1.
E176. The method of any one of E167-E175, wherein the method further comprises evaluating neutrophil levels after administration of the polypeptide of SEQ ID NO: 1.
E177. The method of E157 or E168, wherein the myelodysplastic syndrome is myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)), myelodysplastic syndrome associated with isolated del chromosome abnormality (myelodysplastic syndrome with isolated del (5q)), myelodysplastic syndrome with excess blasts (e.g., myelodysplastic syndrome with excess blasts-type 1 (MDS-EB-1) or myelodysplastic syndrome with excess blasts-type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T).
E178. The method of E177, wherein the myelodysplastic syndrome is MDS-SLD.
E179. The method of E177, wherein the myelodysplastic syndrome is MDS-MLD.
E180. The method of E177, wherein the myelodysplastic syndrome is MDS-RS-SLD.
E181. The method of E177, wherein the myelodysplastic syndrome is MDS-RS-MLD.
E182. The method of E177, wherein the myelodysplastic syndrome is myelodysplastic syndrome with isolated del (5q).
E183. The method of E177, wherein the myelodysplastic syndrome is MDS-EB-1.
E184. The method of E177, wherein the myelodysplastic syndrome is MDS-EB-2.
E185. The method of E177, wherein the myelodysplastic syndrome is MDS-U.
E186. The method of E177, wherein the myelodysplastic syndrome is MDS/MPN-RS-T.
E187. The method of any one of E177-E186, wherein the myelodysplastic syndrome is a ring sideroblast positive myelodysplastic syndrome (RS positive MDS, e.g., the subject has ring sideroblasts).
E188. The method of E187, wherein the RS-positive myelodysplastic syndrome is associated with a splicing factor mutation.
E189. The method of E188, wherein the splicing factor mutation is a mutation in Splicing Factor 3b Subunit 1 (SF3B1).
E190. The method of any one of E177-E179 and E182-E186, wherein the myelodysplastic syndrome is a non-ring sideroblast myelodysplastic syndrome (non-RS, e.g., the subject lacks ring sideroblasts).
E191. The method of any one of E177-E190, wherein the myelodysplastic syndrome is a very low, low, or intermediate risk myelodysplastic syndrome (e.g., as determined by the Revised International Prognostic Scoring System).
E192. The method of E191, wherein the myelodysplastic syndrome is a very low risk myelodysplastic syndrome (e.g., as determined by the Revised International Prognostic Scoring System).
E193. The method of E191, wherein the myelodysplastic syndrome is a low risk myelodysplastic syndrome (e.g., as determined by the Revised International Prognostic Scoring System).
E194. The method of E191, wherein the myelodysplastic syndrome is an intermediate risk myelodysplastic syndrome (e.g., as determined by the Revised International Prognostic Scoring System).
E195. The method of any one of E177-E194, wherein the myelodysplastic syndrome is associated with a defect in terminal maturation.
E196. The method of any one of E177-E195, wherein the myelodysplastic syndrome is associated with a defect in early-stage hematopoiesis (e.g., commitment or differentiation of progenitor cells).
E197. The method of any one of E177-E196, wherein the myelodysplastic syndrome is associated with elevated endogenous erythropoietin levels.
E198. The method of any one of E177-E197, wherein the myelodysplastic syndrome is associated with hypocellular bone marrow (e.g., the subject has hypocellular bone marrow).
E199. The method of any one of E156-E198, wherein the subject does not respond well to treatment with erythropoietin (EPO), is susceptible to the adverse effects of EPO, or does not respond well to treatment with an erythroid maturation agent.
E200. The method of any one of E156-E199, wherein the subject has previously been treated with an erythropoiesis stimulating agent (ESA).
E201. The method of any one of E156-E200, wherein the subject has not previously been treated with an erythropoiesis stimulating agent (ESA).
E202. The method of any one of E156-E201, wherein the subject has a low transfusion burden.
E203. The method of E202, wherein the subject has received 1-3 units of RBCs (1-3 RBC transfusions) within eight weeks prior to starting treatment with the polypeptide of SEQ ID NO: 1.
E204. The method of E202, wherein the subject has received 0 units of RBCs (0 RBC transfusions) within eight weeks prior to starting treatment with the polypeptide of SEQ ID NO: 1.
E205. The method of any one of E156-E201, wherein the subject has a high transfusion burden.
E206. The method of any one of E156-E205, wherein the method reduces the subject's need for a blood transfusion (e.g., reduces transfusion burden).
E207. The method of E79, wherein the subject has or is at risk of developing a disease or condition that can be treated with erythropoietin or an erythropoiesis-stimulating agent.
E208. The method of E79 or E207, wherein the disease or condition that can be treated with erythropoietin or an erythropoiesis-stimulating agent is end-stage renal disease, renal insufficiency, polycythemia, hemochromatosis, a disease or condition associated with dysfunction of endothelial progenitor cells, a disease or condition having an autoimmune or inflammatory component, a neurological disorder or inflammatory brain disease, gastrointestinal dysmotility, a disease of the endocrine system, a disease of the reproductive system, aging, pregnancy, a menstrual disorder, ischemia or an ischemic disorder or condition, hypoxia or a hypoxic disorder or condition, an ulcer, a burn, a wound (e.g., a chronic wound), ischemia-reperfusion injury, asthma, hypertension, a viral disease or infection, a systemic microbial infection, a gastrointestinal disease, arterial sclerosis, cancer, psychosis, a genetic disease, an inflammatory disease, graft-versus-host disease, cardiovascular disease, an allergy, or arthritis.
E209. The method of E208, wherein the disease or condition that can be treated with erythropoietin or an erythropoiesis-stimulating agent is ischemia.
E210. The method of E209, wherein the ischemia is central nervous system ischemia, liver ischemia, renal ischemia, or cardiac ischemia.
E211. The method of E208, wherein the disease or condition that can be treated with erythropoietin or an erythropoiesis-stimulating agent is an ischemic disorder or condition.
E212. The method of E211, wherein the ischemic disorder or condition is occlusive arterial disease, chronic venous insufficiency, circulatory shock (e.g., hemorrhagic, septic, or cardiogenic shock), pulmonary embolism, myocardial infarction, ischemic stroke, acute respiratory failure, chronic heart failure, atherosclerosis, cardiac cirrhosis, macular degeneration, sleep apnea, Raynaud's disease, systemic sclerosis, nonbacterial thrombotic endocarditis, a transient ischemic attack, or ischemia resulting from general anesthesia.
E213. The method of E208, wherein the disease or condition that can be treated with erythropoietin or an erythropoiesis-stimulating agent is a hypoxic disorder or condition.
E214. The method of E213, wherein the hypoxic disorder or condition is a pulmonary disorder (e.g., chronic obstructive pulmonary disease), perinatal hypoxia, severe pneumonia, pulmonary edema, hyaline membrane disease, liver disease, renal disease, cancer, or altitude sickness.
E215. The method of E208, wherein the disease or condition that can be treated with erythropoietin or an erythropoiesis-stimulating agent is a viral disease or infection.
E216. The method of E215, wherein the viral disease or infection is a Hepatitis C virus infection or an HIV infection.
E217. The method of E208, wherein the disease or condition that can be treated with erythropoietin or an erythropoiesis-stimulating agent is a disease or condition associated with dysfunction of endothelial progenitor cells.
E218. The method of E217, wherein the disease or condition associated with dysfunction of endothelial progenitor cells is heart failure, angina pectoris, endotheliosis, reticuloendotheliosis, age-related cardiovascular disorder, coronary heart disease, atherosclerosis, myocardial ischemia, hypercholesterolemia, an ischemic disorder of the extremities, Raynaud's disease, preeclampsia, pregnancy induced hypertension, an endothelium-mediated chronic inflammatory disorder (e.g., inflammation of the vessels), wound healing, chronic renal failure (chronic kidney disease), or acute renal failure (acute kidney failure).
E219. The method of E218, wherein the disease or condition associated with dysfunction of endothelial progenitor cells is chronic renal failure (chronic kidney disease).
E220. The method of E208, wherein the disease or condition that can be treated with erythropoietin or an erythropoiesis-stimulating agent is an autoimmune or inflammatory disease or condition.
E221. The method of E220, wherein the autoimmune or inflammatory disease or condition is acute cerebrovascular injury, acute brain injury, acute cardiovascular injury, arthritis, an autoimmune disease, a stroke, a neurological injury, or immune-mediated inflammation.
E222. The method of E208, wherein the disease or condition that can be treated with erythropoietin or an erythropoiesis-stimulating agent is a neurological disorder or inflammatory brain disease.
E223. The method of E222, wherein the neurological disorder or inflammatory brain disease is a demyelinating disease, epilepsy, spinal cord injury (e.g., an acute spinal cord injury), a complication following traumatic brain injury (e.g., to treat a symptom of the traumatic brain injury, such as hypotension, hypoxemia, brain swelling, headache, neck pain, difficulty remembering, difficulty concentrating, difficulty making decisions, fatigue, a mood change, nausea, photophobia, blurred vision, ear ringing, a loss of sense of taste, and a loss of sense of smell, seizures, coma, muscle weakness, paralysis, or a progressive decline in neurologic function), a chronic inflammatory brain disease, or a neurological disorder associated with a surgery (e.g., thoracoabdominal aortic surgery).
E224. The method of E223, wherein the chronic inflammatory brain disease is a neurodegenerative disease.
E225. The method of E224, wherein the neurodegenerative disease is Alzheimer's disease, Parkinson's disease, Huntington's disease, amyotrophic lateral sclerosis (ALS), or age-related macular degeneration (AMD).
E226. The method of E223, wherein the demyelinating disease is multiple sclerosis, neuromyelitis optica, acute disseminated encephalomyelitis, or transverse myelitis.
E227. The method of E208, wherein the disease or condition that can be treated with erythropoietin or an erythropoiesis-stimulating agent is gastrointestinal dysmotility.
E228. The method of E227, wherein the gastrointestinal dysmotility is associated with an intestinal injury, abdominal trauma, an intestinal inflammatory condition, an intestinal infection, slow transit constipation, post-operative ileus, a neurodegenerative injury, a neurotraumatic injury, a congenital problem, or a malnutrition-malabsorption problem.
E229. The method of E228, wherein the intestinal infection is a bacterial infection (e.g., an infection that leads to sepsis or bacteremia), peritonitis, or ascites.
E230. The method of E228, wherein the intestinal inflammatory condition is inflammatory bowel disease, Crohn's disease, or ulcerative colitis.
E231. The method of E228, wherein the slow transit constipation is chronic constipation, idiopathic constipation, constipation due to post-operative ileus, or constipation caused by opiate use.
E232. The method of E228, wherein the congenital problem is gastroschisis, omphalocele, aganglionic megacolon, Hirschsprung's disease, chronic intestinal pseudo-obstruction, small left colon syndrome, anorectal anomalies, esophageal dysplasia and atresia, ectopic anus, congenital hernias, or internal anal sphincter achalasia.
E233. The method of E228, wherein the malnutrition-malabsorption problem is associated with an intestinal injury, an abdominal trauma, an intestinal inflammatory condition, an intestinal infection, constipation, post-operative ileus, a neurodegenerative injury, a neurotraumatic injury, a congenital problem, Gaucher disease, refeeding syndrome, extremely low birth weight, cancer cachexia, infection, cancer, spinal cord dysfunction, spinal dysraphism, bifida, a tumor, central nervous system dysfunction, peripheral neuropathy, removal part of the gastrointestinal tract, hemorrhage, liver dysfunction, celiac disease, cystic fibrosis, muscular dystrophies, or cerebral palsy.
E234. The method of E208, wherein the disease or condition that can be treated with erythropoietin or an erythropoiesis-stimulating agent is end-stage renal disease.
E235. The method of E208, wherein the disease or condition that can be treated with erythropoietin or an erythropoiesis-stimulating agent is polycythemia.
E236. The method of any one of E207-E225, wherein the polypeptide of SEQ ID NO: 1 is administered to the subject prior to surgery, after stem cell transplantation, prior to or during a space flight, during or after tissue or organ transplantation, to promote the growth of new blood vessels, for granulation tissue formation, for trauma treatment, or for post-vascular graft treatment.
E237. The method of any one of E207-E236, wherein the subject is receiving kidney dialysis.
E238. The method of any one of E207-E237, wherein the subject does not have anemia.
E239. The method of any one of E207-E238, wherein the subject has normal hematopoiesis.
E240. The method of any one of E207-E239, wherein the subject has low serum erythropoietin.
E241. The method of any one of E207-E240, wherein the method increases erythropoietin levels and/or erythropoietin receptor levels in the subject.
E242. The method of any one of E207-E241, wherein the method promotes the growth of new blood vessels and/or the replacement of damaged vascular regions.
E243. The method of any one of E207-E242, wherein the method promotes granulation tissue formation.
E244. The method of any one of E207-E243, wherein the method reduces infiltration of mononuclear cells into the brain of the subject.
E245. The method of any one of E207-E244, wherein the method improves a neurological deficit, reduces axonal damage, reduces neuronal cell death, or reduces glial cell death.
E246. The method of any one of E1-E245, wherein the method reduces or inhibits the binding of activin A, activin B and/or myostatin to their receptors (e.g., their endogenous receptors).
E247. The method of any one of E1-E246, wherein the polypeptide of SEQ ID NO: 1 is administered in an amount of 1.5 mg/kg to 2.5 mg/kg (e.g., 1.5, 1.6, 1.7, 1.75, 1.8, 1.9, 2.0, 2.1, 2.2, 2.25, 2.3, 2.4, or 2.5 mg/kg).
E248. The method of E247, wherein the polypeptide of SEQ ID NO: 1 is administered in an amount of 1.5 mg/kg.
E249. The method of any one of E1-E246, wherein the polypeptide of SEQ ID NO: 1 is administered in an amount of 2.5 mg/kg to 3.5 mg/kg (e.g., 2.5, 2.6, 2.7, 2.75, 2.8, 2.9, 3.0, 3.1, 3.2, 3.25, 3.3, 3.4, or 3.5 mg/kg).
E250. The method of E249, wherein the polypeptide of SEQ ID NO: 1 is administered in an amount of 3 mg/kg.
E251. The method of any one of E1-E246, wherein the polypeptide of SEQ ID NO: 1 is administered in an amount of 3.5 mg/kg to 4.5 mg/kg (e.g., 3.5, 3.6, 3.7, 3.75, 3.8, 3.9, 4.0, 4.1, 4.2, 4.25, 4.3, 4.4, or 4.5 mg/kg).
E252. The method of E251, wherein the polypeptide of SEQ ID NO: 1 is administered in an amount of 4.5 mg/kg.
E253. The method of any one of E1-E252, wherein the polypeptide of SEQ ID NO: 1 is administered subcutaneously.
E254. The method of any one of E1-E253, wherein the polypeptide of SEQ ID NO: 1 is administered as a homodimer.
E255. The method of any one of E1-E254, wherein the method does not cause a vascular complication in the subject.
E256. The method of E255, wherein the method does not increase vascular permeability or leakage.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “extracellular activin receptor type IIB (ActRIIB) variant” refers to a peptide including a soluble, extracellular portion of the single transmembrane receptor, ActRIIB, that has at least one amino acid substitution relative to a wild-type extracellular ActRIIB.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human hair cell).

As used herein, the terms “bone mineral density (BMD),” “bone density,” and “bone mass” refer to a measure of the amount of bone mineral (e.g., calcium) in bone tissue. BMD may be measured by well-established clinical techniques known to one of skill in the art (e.g., by single-1 or dual-energy photon or X-ray absorptiometry). The concept of BMD relates to the mass of mineral per volume of bone, although clinically it is measured by proxy according to optical density per square centimeter of bone surface upon imaging. BMD measurement is used in clinical medicine as an indirect indicator of osteoporosis and fracture risk. In some embodiments, BMD test results are provided as a T-score, where the T-score represents the BMD of a subject compared to the ideal or peak bone mineral density of a healthy 30-year-old adult. A score of 0 indicates that the BMD is equal to the normal reference value for a healthy young adult. Differences between the measured BMD of subject and that of the reference value for a healthy young adult are measured in standard deviations units (SDs). Accordingly, a T-score of between +1 SD and −1 SD may indicate a normal BMD, a T-score of between −1 SD and −2.5 SD may indicate low bone mass (e.g., osteopenia), and a T-score lower than −2.5 SD may indicate osteoporosis or severe osteoporosis. In some embodiments, a polypeptide of SEQ ID NO: 1, a nucleic acid encoding such a polypeptide, or a vector containing such a nucleic acid molecule is administered to a subject in need thereof, wherein the patient has low bone mass (e.g., a T-Score of between −1 SD and −2.5 SD). In some embodiments, a polypeptide of SEQ ID NO: 1, a nucleic acid encoding such a polypeptide, or a vector containing such a nucleic acid molecule is administered to a subject in need thereof, wherein the patient has osteoporosis (e.g., a T-Score of less than −2.5 SD). In some embodiments, administration of a polypeptide of SEQ ID NO: 1, a nucleic acid encoding such a polypeptide, or a vector containing such a nucleic acid molecule treats the subject by increasing their BMD. In some embodiments, administration of a polypeptide of SEQ ID NO: 1, a nucleic acid encoding such a polypeptide, or a vector containing such a nucleic acid molecule increases the BMD of a subject resulting in an increase in the T-Score of the subject (e.g., resulting in an increase in the T-Score of the subject of 0.1 or more, 0.2 or more, 0.3 or more, 0.4 or more, 0.5 or more, 1.0 or more, or 2.0 or more).

As used herein, the term “bone strength” refers to a measurement of bone that is determined by bone quality in addition to bone mineral density. Bone quality is influenced by bone geometry, microarchitecture, and the properties of constituent tissues. Bone strength can be used to assess the bone's risk of fracture.

As used herein, the term “bone disease” refers to a condition characterized by bone damage (e.g., decreased bone mineral density, decreased bone strength, and/or bone loss). Such diseases or conditions may be caused by an imbalance in osteoblast and/or osteoclast activity (e.g., increased bone resorption or reduced bone formation). Bone diseases include osteoporosis (e.g. primary or secondary osteoporosis), osteopenia, osteopetrosis, bone fracture, bone cancer or cancer metastasis-related bone loss (e.g., bone loss associated with multiple myeloma), Paget's disease, renal osteodystrophy, osteogenesis imperfecta, neuromuscular disease-related bone loss, burn-induced bone loss, anorexia-related bone loss, treatment-related bone loss, diet-related bone loss, bone loss associated with the treatment of obesity, low gravity-related bone loss, and immobility-related bone loss.

As used herein, the term “neuromuscular disease-related bone loss” refers to bone loss that occurs in a subject having a neuromuscular disease. Poor bone health is often a significant problem for patients with neuromuscular disease. Deficiency of bone mineral density and increased incidence of bone fractures, for example, are a well-recognized clinical consequence of diseases such as DMD, ALS, and SMA.

As used herein, the terms “bone remodeling” or “bone metabolism” refer to the process for maintaining bone strength and ion homeostasis by replacing discrete parts of old bone with newly synthesized packets of proteinaceous matrix. Bone is resorbed by osteoclasts and is deposited by osteoblasts in a process called ossification. Osteocyte activity plays a key role in this process. Conditions that result in a decrease in bone mass can either be caused by an increase in resorption, or a decrease in ossification. In a healthy individual, during childhood, bone formation exceeds resorption. As the aging process occurs, resorption exceeds formation. Bone resorption rates are also typically much higher in post-menopausal older women due to estrogen deficiency related to menopause.

As used herein, the terms “bone resorption” or “bone catabolic activity” refer to a process by which osteoclasts break down the tissue in bones and release the minerals, resulting in a transfer of the mineral (e.g., calcium) from bone tissue to the blood. Increased rates of bone resorption are associated with aging, including in post-menopausal women. High rates of bone resorption, or rates of bone resorption that exceed the rate of ossification, are associated with bone disorders, such as decreased bone mineral density, including osteopenia and osteoporosis, and can result in bone loss. In some embodiments, a polypeptide of SEQ ID NO: 1, a nucleic acid encoding such a polypeptide, or a vector containing such a nucleic acid molecule is administered to a subject in need thereof to decrease bone resorption (e.g., decrease bone loss) in the subject (e.g., the amount or rate of bone resorption in the subject).

As used herein, the terms “bone formation,” “ossification,” “osteogenesis,” or “bone anabolic activity” refer to the process of forming new bone tissue by osteoblasts. In some embodiments, a polypeptide of SEQ ID NO: 1, a nucleic acid encoding such a polypeptide, or a vector containing such a nucleic acid molecule is administered to a subject in need thereof, to increase bone formation (e.g., increase the amount or rate of bone formation or osteogenesis in the subject). Reduced rates of bone formation, or rates of bone formation that are exceeded by the rate of bone resorption, can result in bone loss.

As used herein, the terms “increasing” and “decreasing” refer to modulating resulting in, respectively, greater or lesser amounts, of function, expression, or activity of a metric relative to a reference. For example, subsequent to administration of a polypeptide of SEQ ID NO: 1 in a method described herein, the amount of a marker of a metric (e.g., bone mineral density) as described herein may be increased or decreased in a subject relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “fibrosis” refers to the pathological process of excess formation of fibrous connective tissue. Fibrosis is characterized by fibroblast accumulation and collagen deposition in excess of normal deposition in any particular tissue. In response to inflammation or an injury to a tissue, nearby fibroblasts can migrate into the wound, proliferate, and produce large amounts of collagenous extracellular matrix. When fibrosis occurs in response to injury, the term “scarring” can be used as synonym. Fibrosis may occur in many tissues of the body, including, e.g., lungs, skin, liver, kidney, heart, eye, tendon, cartilage, pancreatic tissue, uterine tissue, neural tissue, testis, ovary, adrenal gland, artery, vein, bone marrow, colon, small and large intestine, biliary tract, and gut.

As used herein, the terms “pulmonary hypertension” or “PH” refer to a disease characterized by an increase in blood pressure between the heart and lungs, which can include an increase in blood pressure in pulmonary arteries (pulmonary arterial hypertension), pulmonary veins, or pulmonary capillaries. Pulmonary hypertension can have a number of symptoms, shortness of breath (dyspnea), fatigue, swelling (e.g., edema) of the legs, feet, belly (ascites), or neck, chest pain or pressure, racing pulse or heart palpitations, bluish color to lips or skin (cyanosis), dizziness, or fainting. PH also features reduced exercise tolerance and may lead to heart failure.

As used herein, the terms “pulmonary arterial hypertension” or “PAH” refer to a form of pulmonary hypertension characterized by a narrowing or obstruction in the small pulmonary arteries, often caused by scarring, and an increase in pulmonary arterial blood pressure. PAH is also known as WHO Group I PH. PAH can be diagnosed based on an increase in blood pressure in the pulmonary artery mean pulmonary arterial pressure above 25 mmHg at rest, with a normal pulmonary artery capillary wedge pressure. PAH can lead to shortness of breath, dizziness, fainting, and other symptoms, all of which are exacerbated by exertion. PAH can be a severe disease with a markedly decreased exercise tolerance and heart failure. Two major types of PAH include idiopathic PAH (e.g., PAH in which no predisposing factor is identified) and heritable PAH (e.g., PAH associated with a mutation in BMPR2, ALK1, SMAD9, caveolin 1, KCNK3, or EIF2AK4). In 70% of familial PAH cases, mutations are located in the BMPR2 gene. Risk factors for the development of PAH include family history of PAH, drug use (e.g., methamphetamine or cocaine use), infection (e.g., HIV infection or schistosomiasis), cirrhosis of the liver, congenital heart abnormalities, portal hypertension, pulmonary veno-occlusive disease, pulmonary capillary hemangiomatosis, or connective tissue/autoimmune disorders (e.g., scleroderma or lupus).

As used herein, the terms “venous pulmonary hypertension” and “venous PH” refer to a form of pulmonary hypertension that is secondary to left heart disease. Venous PH is also known as WHO Group II PH. Venous PH may be associated with or caused by left ventricular systolic dysfunction (e.g., failure of the left ventricle), left ventricular diastolic dysfunction, valvular heart disease (e.g., mitral valve or aortic valve disease), congenital cardiomyopathy, or congenital/acquired pulmonary venous stenosis.

As used herein, the terms “hypoxic pulmonary hypertension” and “hypoxic PH” refer to a form of pulmonary hypertension that is due to lung disease or chronic hypoxia. This form of PH is also known as WHO Group III PH. Hypoxic PH may be associated with or caused by chronic obstructive pulmonary disease (e.g., emphysema), interstitial lung disease, sleep-disordered breathing (e.g., sleep apnea), lung disease (e.g., pulmonary fibrosis), alveolar hypoventilation disorders, chronic exposure to high altitude, or developmental abnormalities.

As used herein, the terms “thromboembolic pulmonary hypertension” and “thromboembolic PH” refer to a form of pulmonary hypertension that is related to chronic arterial obstruction (e.g., blood clots). Thromboembolic PH is also known as WHO Group IV PH. Thromboembolic PH may be associated with or caused by chronic thromboembolic pulmonary hypertension, or other pulmonary artery obstructions (e.g., pulmonary emboli, angiosarcoma, arteritis, congenital pulmonary artery stenosis, or parasitic infection).

As used herein, the terms “miscellaneous pulmonary hypertension” and “miscellaneous PH” refer to a form of pulmonary hypertension with unclear or multifactorial mechanisms. This form of PH is categorized as WHO Group V PH. Miscellaneous PH may be associated with or caused by a hematologic disease (e.g., chronic hemolytic anemia, sickle cell disease), a systemic disease (e.g., sarcoidosis, pulmonary Langerhans cell histiocytosis, lymphangioleiomyomatosis, neurofibromatosis, or vasculitis), a metabolic disorder (e.g., glycogen storage disease, Gaucher disease, or thyroid diseases), pulmonary tumoral thrombotic microangiopathy, fibrosing mediastinitis, chronic kidney failure, or segmental pulmonary hypertension.

As used herein, the terms “increase platelet levels” and “promote platelet formation” refer to clinically observable metrics, such as platelet counts, and are intended to be neutral as to the mechanism by which such changes occur. The term “low platelet levels” as used herein refers to platelet counts that are below the range of values that is considered normal for the subject's age and gender. The terms “platelet formation” and “platelet production” refer to the generation of platelets, such as the process in which platelets are produced from megakaryocytes.

As used herein, the terms “increase neutrophil levels” and “promote neutrophil formation” refer to clinically observable metrics, such as neutrophil counts, and are intended to be neutral as to the mechanism by which such changes occur. The term “low neutrophil levels” as used herein refers to neutrophil counts that are below the range of values that is considered normal for the subject's age and gender. The terms “neutrophil formation” and “neutrophil production” refer to the generation of neutrophils such as the process in which neutrophils are produced in the bone marrow.

As used herein, the term “anemia” refers to any abnormality in hemoglobin or red blood cells that leads to reduced oxygen levels in the blood. Anemia can be associated with abnormal production, processing, or performance of erythrocytes and/or hemoglobin. The term anemia refers to any reduction in the number of red blood cells and/or level of hemoglobin in blood relative to normal blood levels.

As used herein, the term “normal hematopoiesis” refers to the process by which the components of blood and blood plasma are produced, which includes the formation of red blood cells (erythrocytes), white blood cells (leukocytes, which includes the formation of lymphocytes, neutrophils, eosinophils, basophils, and macrophages), and platelets (thrombocytes). A subject has normal hematopoiesis if the production of these cells is not impaired and the number of these cells in the blood falls within a range accepted as normal by a medical professional. For example, the normal red blood cell (RBC) range for men is 4.7 to 6.1 million cells per microliter (mcL), the normal RBC range for women who are not pregnant is 4.2 to 5.4 million mcL, and the normal RBC range for children is 4.0 to 5.5 million mcL.

As used herein, the term “a disease or condition that can be treated with EPO or an ESA” refers to a disease or condition that is currently treated by administering EPO, recombinant EPO, an EPO mimetic, or another agent that increases EPO or EPO receptor levels, a disease or condition that could be expected to benefit from increasing EPO or EPO receptor levels based on studies performed in cell culture conditions, animal models, or human trials, or a disease or condition that is associated with low serum EPO. Such diseases and conditions include end-stage renal disease, renal insufficiency, kidney dialysis, spinal cord injury, an iron overload disorder (e.g., hemochromatosis), an inflammatory brain disease, gastrointestinal dysmotility, ischemia, and other diseases and conditions as described in U.S. Pat. Nos. 5,013,718, 7,745,387, 8,466,172, 8,729,030, and 10,695,402 and U.S. Patent Application Publication Nos. US20180303903A1 and US20170312268A1, each of which is hereby incorporated by reference.

As used herein, the term “low serum erythropoietin” refers to a level of serum erythropoietin that is below the normal range. Normal levels of erythropoietin range from 4 to 26 milliunits per liter (mU/mL).

“Hypercholesterolemia” is characterized by elevated concentrations of cholesterol in the blood. By far the most common form of primary hypercholesterolemia is polygenic hypercholesterolemia.

Secondary hypercholesterolemias frequently occur in association with diabetes mellitus, nephrotic syndrome, hypothyroidism, and hepatic disorders.

“Endothelium-mediated chronic inflammatory disorders” are disorders or conditions of a human or animal body that derive from a defense response of the body and its tissues to harmful stimuli, with certain signal molecules altering the properties of endothelial cells so that, in concert with the activation of other cell types, leukocytes remain adherent to endothelial cells, finally penetrate into the tissue and there initiate inflammation. One example of an endothelium-mediated inflammation is leukocytic vasculitis. A central part is played in the activation of an endothelium-mediated inflammatory event by the transcription factor NF-κB. Another system leading to the development of endothelial cell-mediated chronic inflammations is the AGE-RAGE system.

“Endotheliosis” refers to degenerative and proliferative endothelial changes associated with non-thrombopenic purpura. “Reticuloendotheliosis” refers to diseases of the reticulohistiocytic system, such as reticulum, reticulosis, reticulohistiocytosis and Hand-Schüller-Christian disease.

“Myocardial ischemia” refers to bloodlessness or hypoperfusion, that is an impairment of the blood supply, of the muscular wall of the heart as a result of inadequate or absent arterial supply of blood.

A “cardiac infarct” or “myocardial infarct” is a necrosis of a localized region of the myocardium, which usually occurs as an acute event complicating chronic coronary heart disease.

“Coronary heart disease” or “ischemic heart disease” is a degenerative coronary disorder which, owing to a constriction or a closure of coronary vessels of the heart, leads to a reduced blood supply to the myocardium.

“Angina pectoris” refers to an acute coronary insufficiency or stenocardia which may be induced by an imbalance of the oxygen supply and oxygen demand associated with coronary heart disease, coronary spasms, impairments of blood flow, cardiac arrhythmias, hypertension, or hypotension.

“Raynaud's disease” refers to ischemic states which are caused by vasoconstriction, that is vessel spasms, and occurs episodically, usually in the arteries of the fingers. Primary Raynaud's disease is a purely functional impairment of the small vessels supplying the distal parts of the extremities, whereas secondary Raynaud's disease has another disease underlying it, for example an inflammation of vessels.

“Preeclampsia” is an endothelial and vascular disease of the maternal body and appears to be the effect of endotheliotropic substances from the placenta. Preeclampsia is a multisystem disorder which may lead to disturbances of function of numerous organs and be manifested by diverse symptoms. The impairments of blood supply which are typical of the disorder are the result of an increased vascular resistance, possibly with local variations in severity. It is regarded as confirmed that an endothelial dysfunction is the central component of the pathogenesis of preeclampsia.

“Renal failure” refers to the restricted ability of the kidneys to excrete substances normally excreted in the urine, and in advanced stages there is also loss of the ability to regulate the electrolyte, water and acid-base balance. Terminal renal failure is characterized by a collapse of the excretory and endocrine function of the kidneys.

“Heart failure” refers to a pathological state that is also referred to as myocardial insufficiency or weakness of the heart muscle. Heart failure is characterized by inadequate functioning of the heart, the heart no longer being capable of efficient delivery to comply with the requirements. Heart failure can be categorized according to various aspects. For example, according to the affected segment of the heart it is classified as right heart failure, left heart failure and failure on both sides (global failure). According to the stability of an equilibrium influenced by physiological and therapeutic mechanisms, a distinction is made between compensated and decompensated heart failure. Classification takes place into acute and chronic heart failure according to the time course. Causes of heart failure are, inter alia, myocardial infarction, cardiomyopathy, inborn or acquired cardiac defects, essential or pulmonary hypertension, cardiac arrhythmias, coronary heart disease or myocarditis.

The term “ischemia” refers to a reduction in blood flow. Ischemia is associated with a reduction in nutrients, including oxygen, delivered to tissues. Ischemia may arise due to conditions such as atherosclerosis, formation of a thrombus in an artery or vein, or blockage of an artery or vein by an embolus, vascular closure due to other causes, e.g., vascular spasm, etc. Such conditions may reduce blood flow, producing a state of hypoperfusion to an organ or tissue, or block blood flow completely. Other conditions that can produce ischemia include tissue damage due to trauma or injury, such as, e.g., spinal cord injury; viral infection, which can lead to, e.g., congestive heart failure, etc. The terms “ischemic conditions” and “ischemic disorders” refer to acute ischemic conditions including myocardial infarction, ischemic stroke, pulmonary embolism, perinatal hypoxia, circulatory shock including, e.g., hemorrhagic, septic, cardiogenic, etc., acute respiratory failure, etc., chronic ischemic conditions including atherosclerosis, chronic venous insufficiency, chronic heart failure, cardiac cirrhosis, macular degeneration, sleep apnea, Raynaud's disease, systemic sclerosis, nonbacterial thrombotic endocarditis, occlusive artery disease, angina pectoris, TIAs, chronic alcoholic liver disease, etc. Ischemic conditions may also result when individuals are placed under general anesthesia and can cause tissue damage in organs prepared for transplant.

The terms “hypoxia” and “hypoxic” refer to an environment with levels of oxygen below normal. Hypoxia may be induced in cells by culturing the cells in a reduced oxygen environment, or cells may be treated with compounds that mimic hypoxia. Determining oxygen levels that define hypoxia in cell culture is well within the skill in the art. The terms “hypoxic conditions” and “hypoxic disorders” include ischemic disorders (ischemic hypoxia) such as those listed above, wherein hypoxia results from reduced circulation; pulmonary disorders (hypoxic hypoxia) such as chronic obstructive pulmonary disease (COPD), severe pneumonia, pulmonary edema, hyaline membrane disease, and the like, wherein hypoxia results from reduced oxygenation of the blood in the lungs; liver or renal disease, cancer or other chronic illness, and altitude sickness, etc.

“Wound healing” refers to the physiological processes for regenerating damaged tissue and for closing a wound, especially formation of new connective tissue and capillaries. The wound healing may be primary wound healing (first intention healing), which is characterized by rapid and complication-free closure and substantially complete recovery as a result of minimal formation of new connective tissue between the edges of a wound, which have a good blood supply and are approximated where appropriate, of a clean wound. Wounds where the edges of the wound are further apart and, in particular, crushed or necrotic, and infected wounds, undergo delayed secondary wound healing (second intention healing) in which, as a result of an (a) bacterial inflammation, there is filling of the tissue defect with granulation tissue and extensive formation of scar tissue. Epithelialization starting from the edge terminates the wound healing. The wound healing is divided into three phases, namely latency phase, proliferative phase and repair phase. The latency phase in turn is divided into the oxidative phase with scab formation, especially in the first few hours after the wound occurred, and the absorptive phase with catabolic autolysis, which extends over a period of from one to three days after the wound occurred. The proliferative phase is characterized by anabolic repair with production of collagen by fibroblasts and occurs on the fourth to seventh day after the wound occurred. The repair phase occurs after the eighth day after the wound occurred and is characterized by transformation of the granulation tissue into a scar.

A “wound” refers to an interruption of the coherence of body tissues with or without loss of substance and caused by mechanical injury or physically caused cell damage. Types of wounds are mechanical wounds, thermal wounds, chemical wounds, radiation wounds and disease-related wounds. Mechanical wounds arise through traumatic violence and occur in particular as incision and puncture wounds, crushing, lacerating, tearing and abrading wounds, scratch and bite wounds and projective wounds. Thermal wounds arise through exposure to heat or cold. Chemical wounds arise in particular through the action of acids or alkalis. Radiation wounds arise for example through exposure to actinic and ionizing radiation. Wounds occurring in relation to disease are in particular congestion-related wounds, traumatic wounds, diabetic wounds, etc.

The term “inflammatory brain disease or disorder” as used herein refers to a brain disease or disorder caused by acute or chronic inflammatory responses in the central nervous system. Acute inflammatory responses in the brain include activation of microglia, appearance of dendritic cells, and the release of pro-inflammatory cytokines and chemokines in the central nervous system. Chronic inflammatory responses include long-standing activation of microglia and subsequent sustained release of inflammatory mediators. Such long-standing activation of microglia results in activation and proliferation of additional microglia, and further release of inflammatory factors. Examples of chronic inflammatory brain diseases or disorders include demyelinating diseases, such as multiple sclerosis, and neurodegenerative diseases, such as Alzheimer's disease (AD), Parkinson's disease (PD), Huntington's disease, amyotrophic lateral sclerosis (ALS), and age-related macular degeneration (AMD).

As used herein, the term “thrombocytopenia” refers to a condition in which the blood contains a lower-than-normal number of platelets, which may be due to a deficiency in platelet production, accumulation of platelets within an enlarged spleen, or the destruction of platelets. Normal blood platelet levels range from about 150,000 to 450,000 per microliter blood in humans. A platelet count of less than 150,000 platelets per microliter is lower than normal. Bleeding can occur after a relatively minor injury if the platelet count falls below 50,000 platelets per microliter of blood, and serious bleeding may occur without any recognized injury if the platelet count falls below 10,000 to 20,000 platelets per microliter of blood.

As used herein, the term “immune thrombocytopenia” is used herein to refer to any type of thrombocytopenia arising from an autoimmune response directed against an individual's own platelets. Immune thrombocytopenia includes primary immune thrombocytopenia, in which autoimmune response is the original cause for the decrease in the platelet counts, such as idiopathic thrombocytopenia purpura. Immune thrombocytopenia also includes secondary immune thrombocytopenia, in which the decrease in platelet counts is associated with one or more other diseases that cause an individual's body to generate an autoimmune response against its own platelets, such as systemic lupus erythematosus (SLE), antiphospholipid syndrome (APS), Evans syndrome, immune thyroid disease, leukemia (e.g., chronic lymphocytic leukemia or large granular T-lymphocyte lymphocytic leukemia), or chronic infection (e.g., with Helicobacter pylori, human immunodeficiency virus (HIV), or Hepatitis C).

As used herein, the term “neutropenia” refers to a condition in which the blood contains an abnormally low number of neutrophils. The typical lower limit of the neutrophil count is about 1500 cells per microliter of blood. Below this level, the risk of infection increases. Neutropenia severity is classified as: mild (1000 to 1500 neutrophils per microliter of blood), moderate (500 to 1000 neutrophils per microliter of blood), and severe (below 500 neutrophils per microliter of blood). Neutropenia has many causes, but they typically fall into two main categories: destruction or depletion of neutrophils faster than the bone marrow can produce new neutrophils, or reduced production of neutrophils in the bone marrow.

As used herein, the term “low transfusion burden” refers to a condition of a subject that has received less than four units of red blood cells (RBCs) within eight weeks (e.g., 3, 2, 1, or 0 units of RBCs within eight weeks) prior to treatment with a polypeptide of SEQ ID NO: 1. A subject with a low transfusion burden can be identified as having anemia based on measurements of mean hemoglobin concentration. A subject with a low transfusion burden and a mean hemoglobin concentration of less than 10.0 g/dL of two measurements performed at least one week apart prior to treatment with a polypeptide of SEQ ID NO: 1 (e.g., one measurement performed within one day prior to treatment and the other performed 7-28 days prior, not influenced by RBC transfusion within seven days of measurement) is defined as having anemia. In some embodiments, a subject with a low transfusion burden receives 1-3 units of RBCs (1-3 RBC transfusions) within eight weeks prior to treatment with a polypeptide of SEQ ID NO: 1. In some embodiments, a subject with a low transfusion burden does not receive any units of RBCs (0 RBC transfusions) within eight weeks prior to treatment with a polypeptide of SEQ ID NO: 1.

As used herein, the term “high transfusion burden” refers to a condition of a subject requiring greater than or equal to four units of RBCs (e.g., 4, 5, 6, 7, 8, or more units) within eight weeks prior to treatment with a polypeptide of SEQ ID NO: 1. A subject with a high transfusion burden can be identified as having anemia based on measurements of mean hemoglobin concentration. A subject with a high transfusion burden and a mean hemoglobin concentration of less than or equal to 9.0 g/dL is defined as having anemia.

As used herein, the term “ineffective hematopoiesis” refers to the failure to produce fully mature hematopoietic cells (e.g., the failure to produce red blood cells, platelets, and neutrophils). Ineffective hematopoiesis may be due to single or multiple defects, such as abnormal proliferation and/or differentiation of progenitor cells (e.g., an excessive production of progenitors that are unable to complete differentiation), that can lead to a hyperproliferation or a shortage of progenitor cells.

As used herein, the terms “erythropoiesis stimulating agent” and “ESA” refer to a class of drugs that act on the proliferation stage of red blood cell development by expanding the pool of early-stage progenitor cells. Examples of erythropoiesis-stimulating agents are epoetin alfa and darbepoetin alfa.

As used herein, the term “metabolic disease” refers to a disease, disorder, or syndrome that is related to a subject's metabolism, such as breaking down carbohydrates, proteins, and fats in food to release energy, and converting chemicals into other substances and transporting them inside cells for energy utilization and/or storage. Some symptoms of a metabolic disease include high serum triglycerides, high low-density cholesterol (LDL), low high-density cholesterol (HDL), and/or high fasting insulin levels, elevated fasting plasma glucose, abdominal (central) obesity, and elevated blood pressure. Metabolic diseases increase the risk of developing other diseases, such as cardiovascular disease. In the present invention, metabolic diseases include, but are not limited to, obesity, Type 1 diabetes, and Type 2 diabetes.

As used herein, the term “treatment-related metabolic disease” refers to a metabolic disease (e.g., obesity, Type 1 diabetes, or Type 2 diabetes) associated with a medication taken by the subject (e.g., a metabolic disease developed during treatment with the medication). The medication can be one that the subject continues to take, or one taken previously that led to the development of metabolic disease. Medications associated with the development of obesity include glucocorticoids (e.g., corticosteroids, such as prednisone), selective serotonin reuptake inhibitors (SSRIs, e.g., paroxetine, mirtazapine, fluoxetine, escitalopram, sertraline), tricyclic antidepressants (e.g., amitriptyline), mood stabilizers (e.g., valproic acid, lithium), antipsychotics (e.g., olanzapine, chlorpromazine, clozapine), and diabetes medication (e.g., insulin, chlorpropamide). Medications associated with the development of diabetes include glucocorticoids (e.g., corticosteroids, which may cause glucocorticoid-induced diabetes mellitus), SSRIs, serotonin-norepinephrine reuptake inhibitors (SNRIs), mood stabilizers (e.g., lithium and valproic acid), and antipsychotics (e.g., olanzapine and clozapine). In some embodiments, the development of obesity may lead to the development of diabetes.

As used herein, the term “age-related metabolic disease” refers to a metabolic disease (e.g., obesity, Type 1 diabetes, or Type 2 diabetes) that develops with age. For example, the risk of diabetes increases with age and is more common in older adults, with approximately 25% of adults over 60 having diabetes. Adults can develop Type 2 diabetes or new-onset Type 1 diabetes. Rates of obesity also increase with age, with the highest rates of obesity in the United States occurring in adults aged 40-59 (with a prevalence of obesity of 45%). Aging also reduces the body's ability to burn fat, leading to increased fat surrounding internal organs.

As used herein, the term “percentage of body weight gain” refers to the percentage of gained body weight compared to a prior body weight of a subject at a prior time. The percentage of body weight gain can be calculated as follows:

100×[(body weight at a later time-body weight at a prior time)/(body weight at a prior time)]

In the present invention, administration of a polypeptide of SEQ ID NO: 1, a nucleic acid molecule encoding the polypeptide, or vector containing such a nucleic acid molecule to a subject can reduce the percentage of body weight gain of the subject.

As used herein, the term “appetite for food intake” refers to a subject's natural desire or need for food. The appetite for food intake of a subject can be monitored by measuring the amount of food consumed after the polypeptide of SEQ ID NO: 1 is administered. In the present invention, administration of a polypeptide of SEQ ID NO: 1, a nucleic acid molecule encoding the polypeptide, or vector containing such a nucleic acid molecule to a subject does not affect the subject's appetite for food intake.

As used herein, the term “adiposity” refers to the fat stored in the adipose tissue of a subject. In the present invention, administration of a polypeptide of SEQ ID NO: 1, a nucleic acid molecule encoding the polypeptide, or vector containing such a nucleic acid molecule to a subject can reduce the subject's adiposity without affecting lean mass.

As used herein, the term “epididymal and perirenal fat pads” refers to the tightly packed fat cells in the epididymis and around the kidney. In the present invention, administration of a polypeptide of SEQ ID NO: 1, a nucleic acid molecule encoding said polypeptide, or vector containing such a nucleic acid molecule to a subject can reduce the weights of epididymal and perirenal fat pads of the subject.

As used herein, the term “fasting insulin” refers to a subject's level of insulin while the subject has not had any food intake for a length of time (i.e., 12-24 hours). Fasting insulin level is used in diagnosing metabolic diseases. Fasting insulin level is also used as an indication of whether a subject is at the risk of developing a metabolic disease. Normally, in a subject suffering from Type 1 diabetes, the subject's fasting insulin level is low compared to that of a healthy subject. In a subject suffering from insulin resistance (i.e., Type 2 diabetes), the subject's fasting insulin level is high compared to that of a healthy subject. In the present invention, administration of a polypeptide of SEQ ID NO: 1, a nucleic acid molecule encoding the polypeptide, or vector containing such a nucleic acid molecule to a subject can modulate the subject's fasting insulin level.

As used herein, the term “rate of glucose clearance” refers to the rate at which glucose is being cleared from the blood. The rate of glucose clearance can be measured in a glucose tolerance test (GTT). In a GTT, a subject is given a certain amount of glucose and blood samples are taken afterward to determine how quickly it is cleared from the blood. The rate of glucose clearance can be used as a parameter in diagnosing and/or determining the risk of developing metabolic diseases such as obesity, diabetes, and insulin resistance.

As used herein, the term “serum lipid profile” refers to the measurement of the distribution of different types of lipids and lipoproteins in a subject's serum. Such measurement can be accomplished by a panel of blood tests. The types of lipids and lipoproteins in a subject's serum include, but are not limited to, cholesterol (e.g., high-density lipoprotein (HDL) and low-density lipoprotein (LDL)), triglyceride, and free fatty acid (FFA). The distribution of the different types of lipids and lipoproteins can be used as a parameter in diagnosing and/or determining the risk of developing metabolic diseases such as obesity, diabetes, and insulin resistance. High levels of cholesterol, especially low-density lipoprotein, are generally regarded as an indication or risk factor for developing certain metabolic diseases, or in some severe medical cases, cardiovascular diseases. In the present invention, administration of a polypeptide of SEQ ID NO: 1, a nucleic acid molecule encoding the polypeptide, or a vector containing such a nucleic acid molecule to a subject improves the subject's serum lipid profile such that the levels of cholesterol (especially low-density lipoprotein) and triglyceride are lowered.

As used herein, the term “serum half-life” refers to, in the context of administering a therapeutic protein to a subject, the time required for plasma concentration of the protein in the subject to be reduced by half. The protein can be redistributed or cleared from the bloodstream, or degraded, e.g., by proteolysis. Serum half-life comparisons can be made by comparing the serum half-life of Fc fusion proteins.

As used herein, the term “lean mass” refers to a component of body composition which includes, e.g., lean mass, body fat, and body fluid. Normally lean mass is calculated by subtracting the weights of body fat and body fluid from total body weight. Typically, a subject's lean mass is between 60% and 90% of totally body weight. In the present invention, administration of a polypeptide of SEQ ID NO: 1, a nucleic acid molecule encoding the polypeptide, or a vector containing such a nucleic acid molecule to a subject increases the subject's lean mass.

As used herein, the term “muscle mass” refers to the primary component of lean mass. Muscle mass can be measured experimentally by measuring muscle weight.

As used herein, the term “muscle disease” refers to a disease or condition involving muscle weakness or atrophy (e.g., skeletal muscle weakness or atrophy). Motor neurons may also be affected in subjects with a muscle disease. A muscle disease may be caused by a genetic mutation (e.g., a muscular dystrophy) or may result from another disease or condition (e.g., cancer cachexia). Muscle diseases include neuromuscular diseases (e.g., a muscular dystrophy, IBM, ALS, SMA, CMT, myasthenia gravis, or multiple sclerosis), sarcopenia, cachexia, disuse atrophy, treatment-related muscle loss or atrophy, hypotonia, muscle loss or atrophy associated with hypoxia, and muscle loss or atrophy associated with a burn injury.

As used herein, the term “neuromuscular disease” refers to a disease that affects voluntary or involuntary muscle function due to problems in the nerves and muscles, typically leading to muscle weakness. Exemplary neuromuscular diseases include amyotrophic lateral sclerosis (ALS), autonomic neuropathy, botulism, Charcot-Marie-Tooth disease (CMT), chronic inflammatory demyelinating polyradiculoneuropathy, congenital myasthenic syndrome, congenital myopathies, cramp-fasciculation syndrome, dermatomyositis, diabetic neuropathy, distal myopathies, dystrophinopathies, endocrine myopathies, focal muscular atrophies, glycogen storage disease type II, Guillain-Barre syndrome, hereditary spastic paraplegia, inclusion body myositis (IBM), Isaac's syndrome, Kearns-Sayre syndrome, Kennedy disease, Lambert-Eaton myasthenic syndrome, metabolic myopathies, metabolic neuropathies, mitochondrial myopathies, motor neuron diseases, multiple sclerosis, muscular dystrophy (e.g., Duchenne (DMD), Becker (BMD), myotonic (DM), facioscapulohumeral (FSHD), limb-girdle (LGMD), distal (DD), oculopharyngeal (OPMD), Emery-Dreifuss (EDMD), and congenital (e.g., MDC1A, MDC1B, MDC1C, FCMD, WWS, RSMD1, MEB, and UCMD)), myasthenia gravis, myotonic dystrophy, necrotizing myopathies, neuromyotonia, neuropathy of Friedreich's Ataxia, nutritional neuropathy, peripheral neuropathy, polymyositis, primary lateral sclerosis, Schwartz-Jampel Syndrome, small fiber neuropathy, spinal and bulbar muscular atrophy, spinal muscular atrophy, spinal muscular atrophy with respiratory distress type 1, spinocerebellar ataxia, stiff person syndrome, toxic neuropathy, and Troyer syndrome. A neuromuscular disease may be inherited in an autosomal dominant or recessive pattern or mutations may occur spontaneously.

As used herein, the phrase “affecting myostatin, GDF-11, activin A, and/or activin B signaling” means changing the binding of myostatin, GDF-11, activin A, and/or activin B to their receptors, e.g., ActRIIA and/or ActRIIB (e.g., endogenous ActRIIB). In some embodiments, a polypeptide including an extracellular ActRIIB variant described herein reduces or inhibits the binding of myostatin, GDF-11, activin A, and/or activin B to their receptors, e.g., ActRIIA and/or ActRIIB (e.g., endogenous ActRIIB).

As used herein, the term “vascular complication” refers to a vascular disorder or any damage to the blood vessels, such as damage to the blood vessel walls. Damage to the blood vessel walls may cause an increase in vascular permeability or leakage. The term “vascular permeability or leakage” refers to the capacity of the blood vessel walls to allow the flow of small molecules, proteins, and cells in and out of blood vessels. An increase in vascular permeability or leakage may be caused by an increase in the gaps (e.g., an increase in the size and/or number of the gaps) between endothelial cells that line the blood vessel walls and/or thinning of the blood vessel walls.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the term “homodimer” refers to a molecular construct formed by two identical macromolecules, such as proteins or nucleic acids. The two identical monomers may form a homodimer by covalent bonds or non-covalent bonds. For example, an Fc domain may be a homodimer of two Fc domain monomers if the two Fc domain monomers contain the same sequence. In another example, a polypeptide described herein including an extracellular ActRIIB variant fused to an Fc domain monomer may form a homodimer through the interaction of two Fc domain monomers, which form an Fc domain in the homodimer.

As used herein, the term “host cell” refers to a vehicle that includes the necessary cellular components, e.g., organelles, needed to express proteins from their corresponding nucleic acids. The nucleic acids are typically included in nucleic acid vectors that can be introduced into the host cell by conventional techniques known in the art (transformation, transfection, electroporation, calcium phosphate precipitation, direct microinjection, etc.). A host cell may be a prokaryotic cell, e.g., a bacterial cell, or a eukaryotic cell, e.g., a mammalian cell (e.g., a CHO cell or a HEK293 cell).

As used herein, the term “therapeutically effective amount” refers an amount of a polypeptide, nucleic acid, or vector of the invention or a pharmaceutical composition containing a polypeptide, nucleic acid, or vector of the invention effective in achieving the desired therapeutic effect in treating a patient having or at risk of developing a disease, such as a muscle disease, a condition involving weakness or atrophy of muscles (e.g., a neuromuscular disease, such as a muscular dystrophy, IBM, ALS, SMA, CMT, myasthenia gravis, or multiple sclerosis; sarcopenia; or cachexia), a disease or condition involving bone damage (e.g., osteoporosis, or a condition involving bone damage, e.g., primary osteoporosis, secondary osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss), a disease or condition involving low platelet levels (e.g., thrombocytopenia), a disease or condition involving low neutrophil levels (e.g., neutropenia), a disease or condition involving fibrosis, a metabolic disease, PH (e.g., PAH, venous PH, hypoxic PH, thromboembolic PH, or miscellaneous PH), or a disease or condition that can be treated with EPO or an ESA (e.g., end-stage renal disease, renal insufficiency, kidney dialysis, spinal cord injury, an iron overload disorder (e.g., hemochromatosis), an inflammatory brain disease, ischemia, or gastrointestinal dysmotility). In particular, the therapeutically effective amount of the polypeptide, nucleic acid, or vector avoids adverse side effects.

As used herein, the term “pharmaceutical composition” refers to a medicinal or pharmaceutical formulation that includes an active ingredient as well as excipients and diluents to enable the active ingredient suitable for the method of administration. The pharmaceutical composition includes pharmaceutically acceptable components that are compatible with the polypeptide, nucleic acid, or vector described herein. The pharmaceutical composition may be in tablet or capsule form for oral administration or in aqueous form for intravenous or subcutaneous administration.

As used herein, the term “pharmaceutically acceptable carrier or excipient” refers to an excipient or diluent in a pharmaceutical composition. The pharmaceutically acceptable carrier must be compatible with the other ingredients of the formulation and not deleterious to the recipient. In the present invention, the pharmaceutically acceptable carrier or excipient must provide adequate pharmaceutical stability to the polypeptide including the polypeptide of SEQ ID NO: 1, the nucleic acid molecule(s) encoding the polypeptide, or a vector containing such nucleic acid molecule(s). The nature of the carrier or excipient differs with the mode of administration. For example, for intravenous administration, an aqueous solution carrier is generally used; for oral administration, a solid carrier is preferred.

As used herein, the term “treating and/or preventing” refers to the treatment and/or prevention of a disease or condition, e.g., a muscle disease (e.g., a neuromuscular disease, such as a muscular dystrophy, IBM, SMA, CMT, ALS, myasthenia gravis, or multiple sclerosis; sarcopenia; or cachexia), a bone disease (e.g., a disease or condition involving bone damage, e.g., osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss), a disease involving low platelet levels (e.g., thrombocytopenia), a disease involving low neutrophil levels (e.g., neutropenia), fibrosis, a metabolic disease, PH (e.g., PAH, venous PH, hypoxic PH, thromboembolic PH, or miscellaneous PH), or a disease or condition that can be treated with EPO or an ESA, such as end-stage renal disease, renal insufficiency, kidney dialysis, spinal cord injury, an iron overload disorder (e.g., hemochromatosis), an inflammatory brain disease, ischemia, or gastrointestinal dysmotility, using methods and compositions of the invention. Generally, treating a muscle, bone, low platelet, low neutrophil, metabolic, or fibrotic disease, PH, or a disease or condition that can be treated with EPO or an ESA occurs after a subject has developed the muscle, bone, low platelet, low neutrophil, metabolic, or fibrotic disease, PH, or the disease or condition that can be treated with EPO or an ESA and/or is already diagnosed with the muscle, bone, low platelet, low neutrophil, metabolic, or fibrotic disease, PH, or the disease or condition that can be treated with EPO or an ESA. Preventing a muscle, bone, low platelet, low neutrophil, metabolic, or fibrotic disease, PH, or a disease or condition that can be treated with EPO or an ESA refers to steps or procedures taken when a subject is at risk of developing the muscle, bone, low platelet, low neutrophil, metabolic, or fibrotic disease, PH, or a disease or condition that can be treated with EPO or an ESA. The subject may show signs or mild symptoms that are judged by a physician to be indications or risk factors for developing the muscle, bone, low platelet, low neutrophil, metabolic, or fibrotic disease, PH, or a disease or condition that can be treated with EPO or an ESA, have another disease or condition associated with the development of the muscle, bone, low platelet, low neutrophil, metabolic, or fibrotic disease, PH, or a disease or condition that can be treated with EPO or an ESA, be undergoing treatment that may cause thrombocytopenia, neutropenia, fibrosis, obesity or diabetes, a disease or condition that can be treated with EPO or an ESA, or loss of bone density (e.g., surgery, chemotherapy, or radiation), or have a family history or genetic predisposition to developing the muscle, bone, low platelet, low neutrophil, metabolic or fibrotic disease, PH, or a disease or condition that can be treated with EPO or an ESA but has not yet developed the disease or condition. As used herein, the term “subject” refers to a human subject.

### DESCRIPTION OF THE DRAWINGS

FIG. 1 is a graph showing that ActRIIB 2.12-Fc elicited dose-dependent reductions in serum FSH in healthy postmenopausal women in Part 2 of the study. In Part 2, maximal suppression was observed at the 4.5 mg/kg dose level with five of six subjects achieving ≥40% reduction in FSH, indicating that treatment maximally inhibited activin signaling.

FIGS. 2A-2B are a series of graphs showing changes in BSAP, a marker of osteoblast activity. Results from Part 2 of the study are shown in FIG. 2A and results from Part 1 of the study are shown in FIG. 2B.

FIG. 3 is a graph showing that serum BSAP increased after administration of each dose of ActRIIB 2.12-Fc. Administration of ActRIIB 2.12-Fc at a 28-day interval resulted in increases in BSAP after each dose in Part 2 (MAD), supportive of activation of osteoblasts after each dose.

FIGS. 4A-4B are a series of graphs showing changes in serum Procollagen Type 1 N-Terminal Propeptide (P1NP) and Osteocalcin after a single dose of ActRIIB 2.12-Fc. Results for P1NP are shown in FIG. 4A and results for osteocalcin are shown in FIG. 4B.

FIGS. 5A-5B are a series of graphs showing that multiple doses of ActRIIB 2.12-Fc did not elicit changes in erythropoiesis. Treatment with three doses of ActRIIB 2.12-Fc at 28-day intervals did not elicit changes in hemoglobin (FIG. 5A) or red blood cells (FIG. 5B).

### DETAILED DESCRIPTION OF THE INVENTION

Described herein are methods for treating or preventing (e.g., preventing or slowing the development of) a disease or condition involving weakness or atrophy of muscles (i.e., a muscle disease), a bone disease, fibrosis, thrombocytopenia, neutropenia, pulmonary hypertension (PH) (e.g., PAH, venous PH, hypoxic PH, thromboembolic PH, or miscellaneous PH), or a metabolic disease (e.g., obesity, Type 1 diabetes, or Type 2 diabetes) by administering to a subject an ActRIIB-Fc polypeptide having the sequence of SEQ ID NO: 1 once every 28 days in an amount of 1.5 mg/kg to 4.5 mg/kg.

### Activin Type II Receptors

There exist two types of activin type II receptors: ActRIIA and ActRIIB. Activin type II receptors are single transmembrane domain receptors that modulate signals for ligands in the transforming growth factor β (TGF-β) superfamily. Ligands in the TGF-β superfamily are involved in a host of physiological processes, such as muscle growth, vascular growth, cell differentiation, homeostasis, and osteogenesis. Examples of ligands in the TGF-β superfamily include, e.g., activin (e.g., activin A and activin B), inhibin, growth differentiation factors (GDFs) (e.g., GDF8, also known as myostatin), and bone morphogenetic proteins (BMPs) (e.g., BMP9).

Myostatin and activins are known to play a role in the regulation of skeletal muscle growth. For example, mice without myostatin show a large increase in skeletal muscle mass. Myostatin has also been implicated in promoting fibrosis. Mice lacking myostatin show a reduction in muscle fibrosis, and injection of myostatin-coated beads induces muscle fibrosis in mice. Mice overexpressing an activin subunit that leads to the production of diffusible activin A also exhibit fibrosis. In addition, activins are expressed abundantly in bone tissues and regulate bone formation by controlling both osteoblast and osteoclast functions. Activin A has been reported to be upregulated in bone disease and inhibits osteoblast activity. Myostatin is also implicated in bone homeostasis through increasing osteogenesis and inhibiting osteoblast activity. TGF-β signaling pathways also regulate hematopoiesis, with signaling pathways involving activins preventing the differentiation of red blood cell, platelet, and neutrophil progenitor cells in order to maintain progenitor cells in a quiescent state, and signaling pathways involving BMPs promoting differentiation of progenitor cells. Homeostasis of this process is essential to ensure that all cell types, including red cells, white cells, and platelets, are properly replenished in the blood. Elevated activin A has also been observed in clinical and experimental pulmonary hypertension. Furthermore, activins are highly expressed in adipose tissue, and increased myostatin levels and activin receptor levels have been observed in subcutaneous and visceral fat of obese mice. Additionally, myostatin has been shown to be elevated in skeletal muscle and plasma of obese and insulin resistant women, and both type I and type II activin receptors have been linked to pancreatic function and diabetes. These data suggest that increased signaling through activin receptors, either due to increased expression of activin receptor ligands (e.g., activin A, activin B, myostatin) or increased expression of activin receptors themselves, could contribute to a variety of diseases and conditions, including muscle atrophy or weakness, fibrosis, bone disease, thrombocytopenia, neutropenia, pulmonary hypertension, and metabolic disease. Methods that reduce or inhibit activin A, activin B, myostatin, and/or GDF-11 signaling could, therefore, be used in the treatment of diseases and conditions involving muscle atrophy or weakness, fibrosis, bone loss or bone damage (e.g., a bone disease), low platelet levels (e.g., thrombocytopenia), low neutrophil levels (e.g., neutropenia), pulmonary hypertension (e.g., PAH, venous PH, hypoxic PH, thromboembolic PH, or miscellaneous PH), or metabolic disorders (e.g., obesity, Type 1 diabetes, or Type 2 diabetes).

The present invention is based, in part, on the discovery that administration of ActRIIB 2.12-Fc, a homodimer of the polypeptide of SEQ ID NO: 1, to healthy post-menopausal women once every 28 days for 12 weeks at a dose of 1.5 mg/kg to 4.5 mg/kg decreased follicle stimulating hormone (FSH) and increased serum bone-specific alkaline phosphatase (BSAP), which is indicative of activin target engagement. These doses were also generally well tolerated. The polypeptide of SEQ ID NO: 1 contains an extracellular ActRIIB variant that binds to and inhibits TGF-β superfamily ligands activin A, activin B, GDF-11, and myostatin fused to a human immunoglobulin G1 Fc domain via a short linker sequence. These data suggest that the polypeptide of SEQ ID NO: 1 or a composition containing said polypeptide can be administered to human subjects once every 28 days at a dose of 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) to treat diseases characterized by increased activin signaling, such as the indications described herein (e.g., bone diseases and PAH). The polypeptide of SEQ ID NO: 1 is provided below:

GRGEAETRECLYYNANWELERTNQSGVERCEGEKDKRLHCYASWRNSSG
SLEIVKKGCWLDDFNCYDRDTCVATKENPQVYFCCCEGNMCNERFTHLP
EAGGPEVTYEPPPTAPTGGGDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSP
GK

The C-terminal Lys345 of the polypeptide of SEQ ID NO: 1 (the C-terminal Lys in the Fc region of SEQ ID NO: 1) may or may not be present, without affecting the structure or stability of the polypeptide. The disclosure specifically contemplates SEQ ID NO: 1 that does not include the C-terminal Lys corresponding to Lys345. The polypeptide of SEQ ID NO: 1 may be expressed including a C-terminal Lys345 which then may be proteolytically cleaved upon expression of the polypeptide (e.g., the polypeptide of SEQ ID NO: 1 is expressed using a nucleic acid construct encoding the polypeptide including a C-terminal lysine residue). The polypeptide of SEQ ID NO: 1 may also be expressed without including the C-terminal Lys345.

### Vectors, Host Cells, and Protein Production

The polypeptide of SEQ ID NO: 1 can be produced from a host cell. A host cell refers to a vehicle that includes the necessary cellular components, e.g., organelles, needed to express the polypeptide described herein from its corresponding nucleic acids. The nucleic acids may be included in a nucleic acid vector that can be introduced into the host cell by conventional techniques known in the art (e.g., transformation, transfection, electroporation, calcium phosphate precipitation, direct microinjection, infection, or the like). The choice of nucleic acid vector depends in part on the host cells to be used. Generally, preferred host cells are of either eukaryotic (e.g., mammalian) or prokaryotic (e.g., bacterial) origin.

### Nucleic Acid Vector Construction and Host Cells

A nucleic acid sequence encoding the amino acid sequence of SEQ ID NO: 1 may be prepared by a variety of methods known in the art. These methods include, but are not limited to, oligonucleotide-mediated (or site-directed) mutagenesis and PCR mutagenesis. A nucleic acid molecule encoding a polypeptide of SEQ ID NO: 1 may be obtained using standard techniques, e.g., gene synthesis. Alternatively, a nucleic acid molecule encoding a wild-type extracellular ActRIIB-Fc may be mutated to include the specific amino acid substitutions found in SEQ ID NO: 1 using standard techniques in the art, e.g., QuikChange™ mutagenesis. Nucleic acid molecules can be synthesized using a nucleotide synthesizer or PCR techniques.

A nucleic acid sequence encoding a polypeptide of SEQ ID NO: 1 may be inserted into a vector capable of replicating and expressing the nucleic acid molecule in prokaryotic or eukaryotic host cells. Many vectors are available in the art and can be used for the purpose of the invention. Each vector may include various components that may be adjusted and optimized for compatibility with the particular host cell. For example, the vector components may include, but are not limited to, an origin of replication, a selection marker gene, a promoter, a ribosome binding site, a signal sequence, a nucleic acid sequence encoding SEQ ID NO: 1, and a transcription termination sequence.

In some embodiments, mammalian cells may be used as host cells. Examples of mammalian cell types include, but are not limited to, human embryonic kidney (HEK) (e.g., HEK293, HEK 293F), Chinese hamster ovary (CHO), HeLa, COS, PC3, Vero, MC3T3, NSO, Sp2/0, VERY, BHK, MDCK, W138, BT483, Hs578T, HTB2, BT20, T47D, NSO (a murine myeloma cell line that does not endogenously produce any immunoglobulin chains), CRL7030, and HsS78Bst cells. In some embodiments, E. coli cells may be used as host cells. Examples of E. coli strains include, but are not limited to, E. coli 294 (ATCC® 31,446), E. coli λ 1776 (ATCC®31,537, E. coli BL21 (DE3) (ATCC®BAA-1025), and E. coli RV308 (ATCC® 31,608). Different host cells have characteristic and specific mechanisms for the posttranslational processing and modification of protein products (e.g., glycosylation). Appropriate cell lines or host systems may be chosen to ensure the correct modification and processing of the polypeptide expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press;

2nd ed. 2004 and Vladimir Voynov and Justin A. Caravella (eds.) Therapeutic Proteins: Methods and Protocols (Methods in Molecular Biology) Humana Press; 2nd ed. 2012.

### Protein Production, Recovery, and Purification

Host cells used to produce the polypeptide of SEQ ID NO: 1 may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels, such as 5 to 10%. The pH of the medium is generally from about 6.8 to 7.4, e.g., 7.0, depending mainly on the host organism. If an inducible promoter is used in the expression vector, protein expression is induced under conditions suitable for the activation of the promoter.

In some embodiments, depending on the expression vector and the host cells used, the expressed protein may be secreted from the host cells (e.g., mammalian host cells) into the cell culture media. Protein recovery may involve filtering the cell culture media to remove cell debris. The proteins may be further purified. A polypeptide of SEQ ID NO: 1 may be purified by any method known in the art of protein purification, for example, by chromatography (e.g., ion exchange, affinity, and size-exclusion column chromatography), centrifugation, differential solubility, or by any other standard technique for the purification of proteins. For example, the protein can be isolated and purified by appropriately selecting and combining affinity columns such as Protein A column (e.g., POROS Protein A chromatography) with chromatography columns (e.g., POROS HS-50 cation exchange chromatography), filtration, ultra-filtration, salting-out and dialysis procedures.

In other embodiments, host cells may be disrupted, e.g., by osmotic shock, sonication, or lysis, to recover the expressed protein. Once the cells are disrupted, cell debris may be removed by centrifugation or filtration. In some instances, a polypeptide can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from influenza hemagglutinin protein (Wilson et al., Cell 37:767, 1984).

Alternatively, the polypeptide of SEQ ID NO: 1 can be produced by the cells of a subject (e.g., a human), e.g., in the context of gene therapy, by administrating a vector (such as a viral vector (e.g., a retroviral vector, adenoviral vector, poxviral vector (e.g., vaccinia viral vector, such as Modified Vaccinia Ankara (MVA)), adeno-associated viral vector, and alphaviral vector)) containing a nucleic acid molecule encoding the polypeptide of the invention. The vector, once inside a cell of the subject (e.g., by transformation, transfection, electroporation, calcium phosphate precipitation, direct microinjection, infection, etc.) will promote expression of the polypeptide, which is then secreted from the cell. If treatment of a disease or disorder is the desired outcome, no further action may be required. If collection of the protein is desired, blood may be collected from the subject and the protein purified from the blood by methods known in the art.

### Pharmaceutical Compositions and Preparations

The polypeptide of SEQ ID NO: 1 may be included in a pharmaceutical composition. In some embodiments, a pharmaceutical composition including a polypeptide of SEQ ID NO: 1 may be used in combination with other agents (e.g., therapeutic biologics and/or small molecules) or compositions in a therapy. In addition to a therapeutically effective amount of the polypeptide, the pharmaceutical composition may include one or more pharmaceutically acceptable carriers or excipients, which can be formulated by methods known to those skilled in the art. In some embodiments, the pharmaceutical composition of includes a nucleic acid molecule (DNA or RNA, e.g., mRNA) encoding the polypeptide of SEQ ID NO: 1, or a vector containing such a nucleic acid molecule.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, arginine, and lysine, and carbohydrates such as glucose, mannose, sucrose, and sorbitol. Pharmaceutical compositions containing the polypeptide of SEQ ID NO: 1 can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

The pharmaceutical composition may be prepared in microcapsules, such as hydroxylmethylcellulose or gelatin-microcapsule and poly-(methylmethacrylate) microcapsule. The pharmaceutical composition may also be prepared in other drug delivery systems such as liposomes, albumin microspheres, microemulsions, nanoparticles, and nanocapsules. Such techniques are described in Remington: The Science and Practice of Pharmacy 22^{nd} edition (2012). The pharmaceutical compositions to be used for in vivo administration must be sterile. This is readily accomplished by filtration through sterile filtration membranes.

The pharmaceutical composition may also be prepared as a sustained-release formulation. Suitable examples of sustained-release preparations include semipermeable matrices of solid hydrophobic polymers containing the polypeptide of SEQ ID NO: 1. Examples of sustained release matrices include polyesters, hydrogels, polylactides, copolymers of L-glutamic acid and y ethyl-L-glutamate, non-degradable ethylene-vinyl acetate, degradable lactic acid-glycolic acid copolymers such as LUPRON DEPOT™, and poly-D-(−)-3-hydroxybutyric acid. Some sustained-release formulations enable release of molecules over a few months, e.g., one to six months, while other formulations release pharmaceutical compositions for shorter time periods, e.g., days to weeks.

The pharmaceutical composition may be formed in a unit dose form as needed. The amount of active component, e.g., a polypeptide of SEQ ID NO: 1, included in the pharmaceutical preparations is such that a suitable dose within the designated range is provided (e.g., a dose within the range of 1.5 mg/kg to 4.5 mg/kg of body weight).

The pharmaceutical composition for gene therapy can be in an acceptable diluent or can include a slow-release matrix in which the gene delivery vehicle is imbedded. If hydrodynamic injection is used as the delivery method, the pharmaceutical composition containing a nucleic acid molecule encoding the polypeptide of SEQ ID NO: 1 or a vector (e.g., a viral vector) containing the nucleic acid molecule is delivered rapidly in a large fluid volume intravenously. Vectors that may be used as in vivo gene delivery vehicle include, but are not limited to, retroviral vectors, adenoviral vectors, poxviral vectors (e.g., vaccinia viral vectors, such as Modified Vaccinia Ankara), adeno-associated viral vectors, and alphaviral vectors.

### Routes, Dosage, and Administration

Pharmaceutical compositions that include the polypeptide of SEQ ID NO: 1 as the therapeutic protein may be formulated for, e.g., intravenous administration, parenteral administration, subcutaneous administration, intramuscular administration, intra-arterial administration, intrathecal administration, or intraperitoneal administration. The pharmaceutical composition may also be formulated for, or administered via, oral, nasal, spray, aerosol, rectal, or vaginal administration. For injectable formulations, various effective pharmaceutical carriers are known in the art. See, e.g., ASHP Handbook on Injectable Drugs, Toissel, 18th ed. (2014).

In some embodiments, a pharmaceutical composition that includes a nucleic acid molecule encoding a polypeptide of SEQ ID NO: 1 or a vector containing such nucleic acid molecule may be administered by way of gene delivery. Methods of gene delivery are well-known to one of skill in the art. Vectors that may be used for in vivo gene delivery and expression include, but are not limited to, retroviral vectors, adenoviral vectors, poxviral vectors (e.g., vaccinia viral vectors, such as Modified Vaccinia Ankara (MVA)), adeno-associated viral vectors, and alphaviral vectors. In some embodiments, mRNA molecules encoding a polypeptide of SEQ ID NO: 1 may be administered directly to a subject.

In some embodiments of the present invention, a nucleic acid molecule encoding the polypeptide of SEQ ID NO: 1 or a vector containing such a nucleic acid molecule may be administered using a hydrodynamic injection platform. In the hydrodynamic injection method, a nucleic acid molecule encoding a polypeptide described herein is put under the control of a strong promoter in an engineered plasmid (e.g., a viral plasmid). The plasmid is often delivered rapidly in a large fluid volume intravenously. Hydrodynamic injection uses controlled hydrodynamic pressure in veins to enhance cell permeability such that the elevated pressure from the rapid injection of the large fluid volume results in fluid and plasmid extravasation from the vein. The expression of the nucleic acid molecule is driven primarily by the liver. In mice, hydrodynamic injection is often performed by injection of the plasmid into the tail vein. In certain embodiments, mRNA molecules encoding a polypeptide of SEQ ID NO: 1 may be administered using hydrodynamic injection.

The dosage of the pharmaceutical compositions of the invention depends on factors including the route of administration, the disease to be treated, and physical characteristics, e.g., age, weight, general health, of the subject. A pharmaceutical composition of the invention may include a dosage of a polypeptide of the invention ranging from 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg). The dosage may be adapted by the physician in accordance with conventional factors such as the extent of the disease and different parameters of the subject.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). Pharmaceutical compositions that include a polypeptide of SEQ ID NO: 1 in an amount of 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) may be administered to a subject in need thereof, for example once every 28 days. Timing between administrations may increase as the medical condition improves or decrease as the health of the patient declines.

### Methods of Treatment

The polypeptide of SEQ ID NO: 1 contains an extracellular ActRIIB variant that can bind to and sequester activin receptor ligands (e.g., activins A and B, myostatin, GDF11) and, therefore, can compete with endogenous activin receptors for ligand binding without activating intracellular signaling pathways. Accordingly, the compositions and methods described herein can be used to treat diseases or conditions in which elevated activin signaling has been implicated in pathogenesis (e.g., diseases or conditions in which increased expression of activin receptors or activin receptor ligands has been observed). For example, myostatin has been implicated in promoting fibrosis, inhibiting skeletal muscle growth, and regulating bone homeostasis, and elevated myostatin has been observed in subcutaneous and visceral fat of obese mice and plasma of obese and insulin resistant women. In addition, activin A has been reported to be upregulated in bone disease, clinical and experimental pulmonary hypertension, adipose tissue, and subcutaneous and visceral fat of obese mice, and has been found to inhibit osteoblast activity and promote fibrosis. Further, both type I and type II activin receptors have been linked to pancreatic function and diabetes. Without wishing to be bound by theory, a therapeutic agent that binds to activin receptor ligands (e.g., GDF11, myostatin, and/or activins) and reduces their binding to or interaction with endogenous activin receptors (e.g., by sequestering the endogenous ligands) may have therapeutic utility for treating or preventing a variety of diseases or conditions, such as a muscle disease, a bone disease, fibrosis, thrombocytopenia, neutropenia, a metabolic disease (e.g., obesity, Type 1 diabetes, or Type 2 diabetes), or PH (e.g., PAH, venous PH, hypoxic PH, thromboembolic PH, or miscellaneous PH). A polypeptide of SEQ ID NO: 1 can also increase EPO and EPO receptor levels. Accordingly, the polypeptide of SEQ ID NO: 1 can be used therapeutically in place of recombinant EPO or an EPO mimetic and can be used to treat any disease or condition that would benefit from increasing EPO and/or EPO receptor levels. This polypeptide can be administered less frequently than current EPO therapies, which would greatly improve convenience for patients and could potentially reduce adverse effects.

The compositions and methods described herein can be used to treat and/or prevent (e.g., prevent the development of or treat a subject diagnosed with) medical conditions, e.g., a muscle disease (e.g., skeletal muscle weakness or atrophy), bone disease, fibrosis, thrombocytopenia (e.g., low platelet count), neutropenia (e.g., low neutrophil count), metabolic disease (e.g., obesity, Type 1 diabetes, or Type 2 diabetes), or PH (e.g., PAH, venous PH, hypoxic PH, thromboembolic PH, or miscellaneous PH). In some embodiments, the polypeptide of SEQ ID NO: 1 may be administered to increase muscle mass and strength in a subject in need thereof. In some embodiments, the polypeptide of SEQ ID NO: 1 may be administered to increase lean mass. The compositions and methods described herein may increase muscle mass and/or lean mass compared to measurements obtained prior to treatment. In some embodiments, the subject may have or be at risk of developing a disease or condition that results in muscle weakness or atrophy (e.g., a neuromuscular disease, cachexia, sarcopenia, or treatment-related muscle loss or atrophy). In some embodiments, the methods described herein are directed to affecting myostatin, GDF-11, activin A, and/or activin B (e.g., reducing or inhibiting the binding of activin A, activin B, GDF-11, and/or myostatin to their endogenous receptors) in a subject having or at risk of developing a disease or condition involving weakness and atrophy of muscles.

In some embodiments, the polypeptide of SEQ ID NO: 1 may be administered to increase bone mineral density, increase bone formation, increase bone strength, reduce the risk or occurrence of bone fracture, or reduce bone resorption in a subject in need thereof. The compositions and methods described herein may increase bone mineral density, increase bone formation, or reduce bone resorption compared to measurements obtained prior to treatment. In some embodiments, the subject may have or be at risk of developing a disease that results in bone damage (e.g., osteoporosis or osteopenia). In some embodiments, the methods described herein are directed to affecting myostatin, GDF-11, activin A, and/or activin B (e.g., reducing or inhibiting the binding of activin A, activin B, GDF-11, and/or myostatin to their endogenous receptors) in a subject having or at risk of developing a disease or condition involving bone damage.

In some embodiments, the polypeptide of SEQ ID NO: 1 may be administered to increase platelet levels (e.g., increase platelet count), promote megakaryocyte differentiation and/or maturation (e.g., to produce platelets), reduce platelet progenitor accumulation, improve blood clotting, reduce bleeding events, reduce bleeding in the skin (e.g., petechiae or bruising), and/or promote or increase platelet formation or production in a subject in need thereof. The compositions and methods described herein may increase platelet levels, promote megakaryocyte differentiation and/or maturation, reduce platelet progenitor accumulation (e.g., by stimulating progenitor cells to progress to maturation), improve blood clotting, reduce bleeding events, reducing bleeding in the skin, and/or promote or increase platelet formation or production compared to measurements obtained prior to treatment. In some embodiments, the subject may have a disease or condition associated with low platelet levels (e.g., thrombocytopenia). In some embodiments, a megakaryocyte can be contacted in vitro with a polypeptide described herein, a nucleic acid encoding the polypeptide, or a vector containing the nucleic acid to generate platelets for the treatment of thrombocytopenia. In some embodiments, the subject may have or be at risk of developing thrombocytopenia (e.g., the subject may have or be at risk of developing thrombocytopenia due to other diseases or conditions, such as a myelodysplastic syndrome, myelofibrosis, myelofibrosis treatment (e.g., treatment with a JAK inhibitor, such as with ruxolitinib or fedratinib), ineffective hematopoiesis, Gaucher disease, aplastic anemia, Fanconi anemia, Diamond Blackfan anemia, Shwachman Diamond syndrome, heavy alcohol consumption, cirrhosis of the liver, cancer (e.g., leukemia or lymphoma), immune thrombocytopenia, an autoimmune disease (e.g., rheumatoid arthritis or lupus (e.g., SLE)), a viral infection (e.g., hepatitis C, HIV, chickenpox, mumps, rubella, parvovirus, or Epstein-Barr virus), a bacterial infection (e.g., bacteremia), a vitamin deficiency (e.g., vitamin B-12 deficiency, folate deficiency, or iron deficiency), cancer treatment (e.g., chemotherapy or radiation therapy), an enlarged spleen, thrombotic thrombocytopeniarpura, idiopathic thrombocytopenia purpura, disseminated intravascular coagulation, hemolytic uremic syndrome, paroxysmal nocturnal hemoglobinuria, acquired amegakaryocytic thrombocytopenia, Pearson syndrome, dyskeratosis congenita, a genetic condition (e.g., Wiskott-Aldrich or May-Hegglin syndrome), dilution of platelets caused by blood transfusion, or a reduction of platelets caused by medication (e.g., heparin, quinine, a sulfa-containing antibiotic, such as vancomycin, rifampin, or trimethoprim, or an anticonvulsant, such as phenytoin)). In some embodiments, the methods described herein are directed to affecting myostatin, GDF-11, activin A, and/or activin B (e.g., reducing or inhibiting the binding of activin A, activin B, GDF-11, and/or myostatin to their endogenous receptors) in a subject having or at risk of developing a disease or condition involving low platelet levels.

In some embodiments, the polypeptide of SEQ ID NO: 1 may be administered to increase neutrophil levels (e.g., increase neutrophil count), increase or promote the differentiation and/or maturation of progenitor cells (e.g., myeloid progenitors, myeloblasts, or myelocytes) into neutrophils, and/or promote or increase neutrophil formation or production in a subject in need thereof. The compositions and methods described herein may increase neutrophil levels, increase or promote the differentiation and/or maturation of progenitor cells into neutrophils, and/or promote or increase neutrophil formation or production compared to measurements obtained prior to treatment. In some embodiments, the subject may have a disease or condition associated with low neutrophil levels (e.g., neutropenia). In some embodiments, the subject may have or be at risk of developing neutropenia (e.g., the subject may have or be at risk of developing neutropenia due to another disease or condition, such as a myelodysplastic syndrome, myelofibrosis, ineffective hematopoiesis, aplastic anemia, Fanconi anemia, Diamond Blackfan anemia, Shwachman Diamond syndrome, paroxysmal nocturnal hemoglobinuria, Pearson syndrome, dyskeratosis congenita, cancer (e.g., leukemia), a vitamin deficiency (e.g., B-12 deficiency or folate deficiency), an enlarged spleen, an autoimmune disease (e.g., granulomatosis with polyangiitis, lupus (e.g., SLE), Evans syndrome, Felty syndrome, Crohn's disease, or rheumatoid arthritis), a viral infection (e.g., chickenpox, Epstein-Barr, Hepatitis A, Hepatitis B, Hepatitis C, HIV/AIDS, cytomegalovirus, Dengue fever, or measles), a bacterial infection (e.g., tuberculosis, salmonella infection, or sepsis), cancer treatment (e.g., chemotherapy or radiation therapy), or treatment with another medication (e.g., medication used to treat overactive thyroid, such as methimazole and propylthiouracil; an antibiotic, such as vancomycin, penicillin G, trimethoprim, and oxacillin; an antiviral drug, such as ganciclovir and valganciclovir; an anti-inflammatory medication for ulcerative colitis or rheumatoid arthritis, such as sulfasalazine; a drug used to treat irregular heart rhythms, such as quinidine and procainamide; an anticonvulsant, such as phenytoin and valproate; an antipsychotic, such as clozapine; or levamisole). In some embodiments, the methods described herein are directed to affecting myostatin, GDF-11, activin A, and/or activin B (e.g., reducing or inhibiting the binding of activin A, activin B, GDF-11, and/or myostatin to their endogenous receptors) in a subject having or at risk of developing a disease or condition involving low neutrophil levels.

In some embodiments, the polypeptide of SEQ ID NO: 1 may be administered to prevent or reduce fibrosis in a subject in need thereof. In some embodiments, the polypeptide may be administered to slow or stop the progression of fibrosis, to reduce the risk of developing fibrosis, or to reduce (e.g., reduce the frequency or severity of) one or more symptom of fibrosis. The compositions and methods described herein may reduce fibrosis or slow the progression of fibrosis by at least compared to the progression of fibrosis prior to treatment or compared to the progression of fibrosis in untreated subjects. In some embodiments, the subject may have or be at risk of developing fibrosis (e.g., the subject may have a disease or condition associated with fibrosis, such as a wound, hepatitis B or C, fatty liver disease, kidney disease (e.g., chronic kidney disease), heart disease, or atherosclerosis, or may be undergoing treatment associated with the development of fibrosis, such as chemotherapy, radiation, or surgery). In some embodiments, the compositions and methods described herein prevent or delay the development of fibrosis in a subject at risk of developing fibrosis (e.g., a subject being treated with chemotherapy, radiation, or surgery, or a subject having a disease or condition associated with fibrosis, such as a wound, hepatitis B or C, fatty liver disease, kidney disease (e.g., chronic kidney disease), heart disease, or atherosclerosis). In some embodiments, the methods described herein are directed to affecting myostatin, GDF-11, activin A, and/or activin B (e.g., reducing or inhibiting the binding of activin A, activin B, GDF-11, and/or myostatin to their endogenous receptors) in a subject having or at risk of developing fibrosis or a disease or condition associated with fibrosis.

In some embodiments, the polypeptide of SEQ ID NO: 1 may be administered to treat PH, reduce PH (e.g., reduce the severity or frequency of one or more symptoms of PH, such as shortness of breath (dyspnea), fatigue, swelling (e.g., edema) of the legs, feet, belly (ascites), or neck, chest pain or pressure, racing pulse or heart palpitations, bluish color to lips or skin (cyanosis), dizziness, or fainting), prevent (e.g., prevent the development of) PH, reduce the risk of developing PH, or slow or stop the progression of PH in a subject in need thereof. In some embodiments, the polypeptide of SEQ ID NO: 1 may be administered to reduce pulmonary vascular resistance, improve performance in the 6-minute walk test (i.e., increase 6-minute walk distance), improve WHO/NYHA FC, improve one or more of mPAP, cardiac output (CO), cardiac index (CI), PAWP, right atrial pressure (RAP), mixed venous oxygen saturation (SvO2), stroke volume (SV), stroke volume index (SVI), and pulmonary artery compliance (PAC), decrease NT-proBNP, attenuate clinical worsening, improve risk stratification measures, improve physical activity (overall activity), or improve health-related quality of life (HRQOL) compared to baseline pre-treatment measurements. The compositions and methods described herein may reduce the symptoms of PH or slow the progression of PH compared to the symptoms or progression observed prior to treatment or compared to symptoms or progression of PH in untreated subjects. In some embodiments, the subject may have or be at risk of developing PH (e.g., the subject may have idiopathic PAH; the subject may have a disease or condition associated with PAH (e.g., a disease or condition that leads to increased risk of developing PAH), such as HIV infection, schistosomiasis, portal hypertension, pulmonary veno-occlusive disease, pulmonary capillary hemangiomatosis, cirrhosis of the liver, a congenital heart abnormality, a connective tissue/autoimmune disorder (e.g., scleroderma, lupus (systemic lupus erythematosus), mixed connective tissue disease, Sjogren syndrome, an inflammatory idiopathic myopathy, or rheumatoid arthritis), a history of drug use or abuse (e.g., methamphetamine or cocaine use), exposure to a toxin, or congenital systemic-pulmonary intracardiac shunt); the subject may have a family history of PH (e.g., heritable PAH); the subject may have a disease or condition associated with venous PH (e.g., a disease or condition that leads to increased risk of developing venous PH), such as left ventricular systolic dysfunction, left ventricular diastolic dysfunction, valvular heart disease, congenital cardiomyopathy, or congenital/acquired pulmonary venous stenosis; the subject may have a disease or condition associated with hypoxic PH (e.g., a disease or condition that leads to increased risk of developing hypoxic PH), such as chronic obstructive pulmonary disease (e.g., emphysema), interstitial lung disease, sleep-disordered breathing (e.g., sleep apnea), lung disease (e.g., pulmonary fibrosis), an alveolar hypoventilation disorder, chronic exposure to high altitude, or a developmental abnormality; the subject may have a disease or condition associated with thromboembolic PH (e.g., a disease or condition that leads to increased risk of developing thromboembolic PH), such as chronic thromboembolic pulmonary hypertension, or a pulmonary artery obstruction (e.g., a pulmonary embolism, angiosarcoma, arteritis, congenital pulmonary artery stenosis, or parasitic infection); or the subject may have a disease or condition associated with miscellaneous PH (e.g., a disease or condition that leads to increased risk of developing miscellaneous PH), such as a hematologic disease (e.g., chronic hemolytic anemia, sickle cell disease), a systemic disease (e.g., sarcoidosis, pulmonary Langerhans cell histiocytosis, lymphangioleiomyomatosis, neurofibromatosis, or vasculitis), a metabolic disorder (e.g., glycogen storage disease, Gaucher disease, or a thyroid disease), pulmonary tumoral thrombotic microangiopathy, fibrosing mediastinitis, chronic kidney failure, or segmental pulmonary hypertension (pulmonary hypertension restricted to one or more lobes of the lungs)). In some embodiments, the compositions and methods described herein prevent or delay the development of PH in a subject at risk of developing PH (e.g., a subject with a family history of PH (e.g., heritable PAH), or a subject having a disease or condition that leads to increased risk of developing PAH, venous PH, hypoxic PH, thromboembolic PH, or miscellaneous PH. In some embodiments, the methods described herein are directed to affecting myostatin, GDF-11, activin A, and/or activin B (e.g., reducing or inhibiting the binding of activin A, activin B, GDF-11, and/or myostatin to their endogenous receptors) in a subject having or at risk of developing PH or a disease or condition associated with PH. In some embodiments, the PH is PAH, venous PH, hypoxic PH, thromboembolic PH, or miscellaneous PH. In some embodiments, the PH is PAH.

In some embodiments, the polypeptide of SEQ ID NO: 1 may be administered to reduce body fat (e.g., amount of body fat or body fat percentage), reduce body weight or body weight gain, reduce fasting insulin levels, increase glucose clearance, reduce LDL, reduce triglycerides, improve serum lipid profile, or increase insulin sensitivity (e.g., reduce in insulin resistance) in a subject in need thereof. The compositions and methods described herein may reduce body fat (e.g., amount of body fat or body fat percentage), reduce body weight or body weight gain, reduce fasting insulin levels, increase glucose clearance, reduce LDL, reduce triglycerides, improve serum lipid profile, or increase insulin sensitivity (e.g., reduce in insulin resistance) compared to measurements obtained prior to treatment. In some embodiments, the subject may have a disease or condition associated with obesity or diabetes (e.g., Type 1 or Type 2 diabetes). In some embodiments, the subject may have or be at risk of developing a metabolic disease (e.g., obesity, Type 1 diabetes, or Type 2 diabetes, e.g., the subject may be overweight, have a family history of obesity, have other medical conditions or risk factors linked to increased risk of developing obesity or diabetes (e.g., advanced age, or treatment with a glucocorticoid, selective serotonin reuptake inhibitor (SSRI), tricyclic antidepressant, mood stabilizer, antipsychotic, serotonin-norepinephrine reuptake inhibitor (SNRI)), have a family history of diabetes, or have prediabetes). In some embodiments, the methods described herein are directed to affecting myostatin, GDF-11, activin A, and/or activin B (e.g., reducing or inhibiting the binding of activin A, activin B, GDF-11, and/or myostatin to their endogenous receptors) in a subject having or at risk of developing a metabolic disease (e.g., obesity, Type 1 diabetes, or Type 2 diabetes).

In some embodiments, a polypeptide of SEQ ID NO: 1 reduces or inhibits the binding of myostatin, activin A, activin B, and/or GDF-11 to their endogenous receptors, e.g., ActRIIA and/or ActRIIB. The polypeptide may reduce the binding of myostatin, activin A, activin B, and/or GDF-11 to their endogenous receptors compared to the binding of myostatin, activin A, activin B, and/or GDF-11 to their endogenous receptors in the absence of the polypeptide. In some embodiments, affecting myostatin, activin A, activin B, and/or GDF-11 signaling (e.g., reducing or inhibiting the binding of myostatin, activin A, activin B, and/or GDF-11 to their endogenous receptors, e.g., ActRIIA and/or ActRIIB) results in an increase in the subject's muscle mass, an increase in the subject's lean mass, an increase in the subject's bone mineral density or bone formation, a decrease in the subject's bone resorption, an increase in the subject's platelet levels (e.g., an increase in platelet count, megakaryocyte differentiation and/or maturation, and/or platelet formation or production), a reduction in the accumulation of platelet progenitor cells, an improvement in blood clotting, a reduction in bleeding events, reduced bleeding in the skin, an increase in the subject's neutrophil levels (e.g., an increase in neutrophil count, e.g., an increase in neutrophil production or formation), an increase in the differentiation and/or maturation of progenitor cells into neutrophils, a reduction in the subject's fibrosis or risk of developing fibrosis, a delay in the development of fibrosis, a reduction (e.g., slowing or inhibiting) in the progression of fibrosis, a reduction body fat (e.g., amount of body fat or body fat percentage), a reduction in body weight or body weight gain, a reduction in fasting insulin levels, an increase in glucose clearance, an improvement in serum lipid profile, an increase in insulin sensitivity (e.g., a reduction in insulin resistance), a reduction in the symptoms of PH, a reduction in the risk of developing PH, a delay in the development of PH, and/or a reduction (e.g., slowing or inhibiting) in the progression of PH. The PH can be PAH, venous PH, hypoxic PH, thromboembolic PH, or miscellaneous PH.

In some embodiments, the polypeptide of SEQ ID NO: 1 may be administered to a subject to increase muscle mass or strength, to increase lean mass, to increase bone mineral density, to increase bone formation, to increase bone strength, to reduce the risk or occurrence of bone fracture, to decrease bone resorption, to increase the subject's platelet levels, to increase megakaryocyte differentiation and/or maturation, to increase platelet formation or production, to reduce the accumulation of platelet progenitor cells, to improve blood clotting, to reduce bleeding events, to reduce bleeding in the skin, to increase the subject's neutrophil levels, to increase neutrophil production or formation, to increase or promote the differentiation and/or maturation of progenitor cells into neutrophils, to prevent or reduce fibrosis (e.g., to reduce fibrosis, to prevent or delay the development of fibrosis, or to slow or stop the progression of fibrosis), to treat metabolic disease, to reduce body fat (e.g., amount of body fat or body fat percentage), to reduce body weight or body weight gain, to reduce fasting insulin levels, to increase glucose clearance, to improve serum lipid profile, to prevent or treat PH (e.g., to reduce symptoms of PH, to prevent or delay the development of PH, or to slow or stop the progression of PH), or to affect myostatin, activin A, activin B, and/or GDF-11 signaling in the subject. The compositions and methods described herein may increase muscle mass or strength, increase lean mass, increase bone mineral density, increase bone formation, increase bone strength, reduce the risk or occurrence of bone fracture, decrease bone resorption, increase the subject's platelet levels, increase megakaryocyte differentiation and/or maturation, increase platelet formation or production, reduce the accumulation of platelet progenitor cells, improve blood clotting, reduce bleeding events, reduce bleeding in the skin, increase the subject's neutrophil levels, increase or promote the differentiation and/or maturation of progenitor cells into neutrophils, increase neutrophil production or formation, prevent or reduce fibrosis, treat metabolic disease, reduce body fat (e.g., amount of body fat or body fat percentage), reduce body weight or body weight gain, reduce fasting insulin levels, increase glucose clearance, improve serum lipid profile, prevent or treat PH, or affect myostatin, activin A, activin B, and/or BMP9 signaling compared to measurements obtained prior to treatment or compared to measurements obtained from untreated subjects having the same disease or condition. In some embodiments, the methods described herein do not cause any vascular complications in the subject, such as increased vascular permeability or leakage.

The invention also includes methods of treating a subject having or at risk of developing a disease or condition involving weakness or atrophy of muscles by administering to the subject an effective amount of a polypeptide of SEQ ID NO: 1. In any of the methods described herein, a subject having or at risk of developing a disease or condition involving weakness or atrophy of muscles has or is at risk of developing a disease or condition including a neuromuscular disease (e.g., a muscular dystrophy, IBM, SMA, CMT, ALS, myasthenia gravis, or multiple sclerosis), sarcopenia, cachexia (e.g., cancer cachexia, HIV-related cachexia, cardiac cachexia (e.g., cachexia associated with heart failure), cachexia associated with chronic kidney disease, or pulmonary cachexia (e.g., cachexia associated with COPD)), disuse atrophy; treatment related muscle loss or atrophy (e.g., glucocorticoid treatment, FGF-21 treatment, GLP-1 treatment, bariatric surgery, cancer therapy, or treatment for obesity or Type 2 diabetes), hypotonia, hypoxia, or muscle loss or atrophy associated with a burn injury. Muscular dystrophies include Duchenne muscular dystrophy (DMD), facioscapulohumeral muscular dystrophy (FSHD), Becker muscular dystrophy (BMD), myotonic dystrophy (DM), congenital muscular dystrophy, limb-girdle muscular dystrophy (LGMD), distal muscular dystrophy (DD), oculopharyngeal muscular dystrophy (OPMD), and Emery-Dreifuss muscular dystrophy (EDMD). There are thirty three types of congenital muscular dystrophies, which include congenital muscular dystrophy type 1A (MDC1A, associated with mutations in laminin alpha 2), congenital muscular dystrophy type 1C (MDC1C, associated with mutations in FKRP), congenital muscular dystrophy type 1D (MDC1D, associated with mutations in LARGE), congenital muscular dystrophy type 1B (MDC1B), Fukuyama congenital muscular dystrophy (FCMD, associated with mutations in fukutin), muscle-eye-brain disease (MEB, which may be associated with mutations in POMGnT1), Walker-Warburg Syndrome (WWS, associated with mutations in B3GNT1 (MDDGA type), POMT1 (MDDGA1 type), POMT2 (MDDGA2 type), ISPD (MDDGA7 type), GTDC2 (MDDGA8 type), TMEM5 (MDDGA10 type), B3GALNT2 (MDDGA11 type), or SGK196 (MDDGA12 type)), rigid spine muscular dystrophy (RSMD1, associated with a mutation in SEPN1), Ullrich congenital muscular dystrophy (UCMD, associated in mutations in COLGA1, COL6A2, or COL6A3), and muscular dystrophies associated with mutations in integrin alpha 7, integrin alpha 9, DOK7, laminin A/C, SBP2, or choline kinase beta. In some embodiments, the methods described herein increase muscle mass, e.g., increase muscle mass, lean mass, and/or muscle strength, e.g., increase muscle mass, lean mass, and/or muscle strength compared to measurements obtained prior to treatment or compared to measurements typically observed in untreated subjects having the same disease or condition. In some embodiments, the muscle is skeletal muscle. In some embodiments, the subject is identified as having a disease or condition that results in muscle weakness or atrophy prior to treatment with the compositions and methods described herein. In some embodiments, the method includes a step of identifying the subject as having a disease or condition that results in muscle weakness or atrophy (e.g., by evaluating lean mass, muscle mass, or strength or by genetic testing for congenital muscular dystrophy) prior to treatment with a polypeptide described herein. The method can further include evaluating lean mass, muscle mass, or strength after administration of a polypeptide of SEQ ID NO: 1 (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing a bone disease by administering to the subject an effective amount of a polypeptide of SEQ ID NO: 1. In any of the methods described herein, a subject having or at risk of developing a bone disease (e.g., bone damage) has or is at risk of developing a disease or condition including osteoporosis (e.g., primary or secondary osteoporosis), osteopenia, osteopetrosis, osteogenesis imperfecta, bone fracture, bone cancer or cancer metastasis-related bone loss, Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, or immobility-related bone loss. In some embodiments, the primary osteoporosis is age-related or hormone-related osteoporosis (e.g., related to a decline in estrogen). In some embodiments, the secondary osteoporosis is immobilization-induced or glucocorticoid-induced (e.g., corticosteroid-induced) osteoporosis. Secondary osteoporosis may also result from endocrinopathies (e.g., Cushing's syndrome, thyrotoxicosis, hyperthyroidism, hypogonadism, hypopituitarism, primary hyperparathyroidism, diabetes mellitus, eating disorders, growth hormone deficiency, and acromegaly), gastrointestinal disorders (e.g., primary biliary cirrhosis, malabsorption syndrome, celiac disease, inflammatory bowel disease, gastric bypass surgery, hemochromatosis, and chronic liver diseases), hematological disorders (e.g., monoclonal gammopathy of uncertain significance, multiple myeloma, systemic mastocytosis, and beta thalassemia major), autoimmune disorders (e.g., rheumatoid arthritis, systemic lupus erythematosus, ankylosing spondylitis, and multiple sclerosis), renal disease (e.g., renal tubular acidosis, chronic kidney disease), medications (e.g., thyroid hormone, aromatase inhibitors, medroxyprogesterone acetate, GnRH agonists and antagonists, selective serotonin reuptake inhibitors, carbamazepine, phenytoin, cyclosporine, tacrolimus, antiretroviral therapy, lithium, heparin, furosemide and proton pump inhibitors), alcoholism, and transplantation. In some embodiments, the bone cancer is multiple myeloma or the cancer metastasis-related bone loss is caused by multiple myeloma. In some embodiments, the treatment-related bone loss occurs due to treatment with FGF-21 or GLP-1, due to treatment with an FGF-21 or GLP-1 containing therapeutic, due to treatment of Type 2 diabetes and/or obesity, due to bariatric surgery, due to androgen or estrogen deprivation therapy, or due to cancer therapy (e.g., chemotherapy or radiation). In some embodiments, the diet-related bone loss is rickets (e.g., vitamin D deficiency). In some embodiments, the low-gravity related bone loss is lack of load-related bone loss. In some embodiments, the methods described herein increase bone mineral density (e.g., increase bone mass), reduce bone resorption (e.g., reduce bone catabolic activity), increase bone formation (e.g., increase bone anabolic activity or increase osteogenesis), increase osteoblast activity or osteoblastogenesis, and/or decrease osteoclast activity or osteoclastogenesis, e.g., increase bone mineral density, reduce bone resorption, increase bone formation, increase osteoblast activity or osteoblastogenesis, and/or decrease osteoclast activity or osteoclastogenesis compared to measurements obtained prior to treatment or compared to measurements typically observed in untreated subjects having the same disease or condition. In some embodiments, the bone is cortical or trabecular bone. In some embodiments, the subject is identified as having a bone disease prior to treatment with the compositions described herein. In some embodiments, the method includes a step of identifying the subject as having a bone disease prior to treatment with a polypeptide described herein. The method can further include evaluating bone mineral density, bone formation, or bone resorption after administration of a polypeptide described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing thrombocytopenia by administering to the subject an effective amount of a polypeptide of SEQ ID NO: 1. In any of the methods described herein, a subject having or at risk of developing low platelet levels (e.g., low platelet counts) has or is at risk of developing thrombocytopenia. In some embodiments, the thrombocytopenia is associated with a bone marrow defect, a myelodysplastic syndrome, bone marrow transplantation, myelofibrosis, myelofibrosis treatment (e.g., treatment with a JAK inhibitor, such as with ruxolitinib or fedratinib), ineffective hematopoiesis, Gaucher disease, aplastic anemia, Fanconi anemia, Diamond Blackfan anemia, Shwachman Diamond syndrome, heavy alcohol consumption, cirrhosis of the liver, cancer (e.g., leukemia or lymphoma), an autoimmune disease (e.g., rheumatoid arthritis, lupus (e.g., SLE), antiphospholipid syndrome (APS), Evans syndrome, or immune thyroid disease), a viral infection (e.g., hepatitis C, HIV, chickenpox, mumps, rubella, parvovirus, or Epstein-Barr virus), a bacterial infection (e.g., bacteremia), an enlarged spleen, a vitamin deficiency (e.g., vitamin B-12 deficiency, folate deficiency, or iron deficiency), cancer treatment (e.g., chemotherapy or radiation therapy), thrombotic thrombocytopenia purpura, idiopathic thrombocytopenia purpura, disseminated intravascular coagulation, hemolytic uremic syndrome, paroxysmal nocturnal hemoglobinuria, or a reduction of platelets caused by medication (medication-induced thrombocytopenia, e.g., thrombocytopenia caused by treatment with heparin, quinine, a sulfa-containing antibiotic, such as vancomycin, rifampin, or trimethoprim, or an anticonvulsant, such as phenytoin)), dilution of platelets caused by blood transfusion, hematopoietic stem cell transplantation, acquired amegakaryocytic thrombocytopenia, Pearson syndrome, dyskeratosis congenita, or contraindication to transfusion (e.g., patients of advanced age, patients with allo-or auto-antibodies, pediatric patients, patients with cardiopulmonary disease, patients who object to transfusion for religious reasons (e.g., some Jehovah's Witnesses)). The myelodysplastic syndrome may be myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)), myelodysplastic syndrome associated with isolated del chromosome abnormality (MDS with isolated del(5q)), myelodysplastic syndrome with excess blasts (MDS-EB; which includes myelodysplastic syndrome with excess blasts—type 1 (MDS-EB-1) and myelodysplastic syndrome with excess blasts—type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T). The myelodysplastic syndrome may be a very low, low, or intermediate risk MDS as determined by the Revised International Prognostic Scoring System (IPSS-R). The myelodysplastic syndrome may be an RS-positive myelodysplastic syndrome (e.g., the subject with a myelodysplastic syndrome may have ring sideroblasts) or a non-RS myelodysplastic syndrome (e.g., the subject with a myelodysplastic syndrome may lack ring sideroblasts). In some embodiments, the RS-positive myelodysplastic syndrome is associated with a splicing factor mutation, such as a mutation in SF3B1. In some embodiments, the MDS is associated with a defect in terminal maturation (often observed in RS-positive MDS and in subjects having splicing factor mutations). In some embodiments, the MDS is associated with a defect in early-stage hematopoiesis (e.g., commitment or early differentiation). In some embodiments, the MDS is associated with elevated endogenous erythropoietin levels. In some embodiments, the myelodysplastic syndrome is associated with hypocellular bone marrow (e.g., the subject with MDS has hypocellular bone marrow). The subject may have a low transfusion burden or a high transfusion burden. In some embodiments, the subject has a low transfusion burden and received 1-3 RBC units in the eight weeks prior to treatment with a polypeptide described herein. In some embodiments, the subject has a low transfusion burden and did not receive a transfusion (received 0 RBC units) in the eight weeks prior to treatment with a polypeptide described herein. In some embodiments, the subject does not respond well to erythropoietin (EPO) or is susceptible to adverse effects of EPO (e.g., hypertension, headaches, vascular thrombosis, influenza-like syndrome, obstruction of shunts, and myocardial infarction). The compositions and methods described herein can also be used to treat subjects that do not respond to an erythroid maturation agent. In some embodiments, the subject has previously been treated with an ESA.

In some embodiments, the subject has not previously been treated with an ESA. In some embodiments, the thrombocytopenia is familial thrombocytopenia (also referred to as inherited thrombocytopenia, e.g., thrombocytopenia associated with a genetic mutation, such as May-Hegglin anomaly, Sebastian syndrome, Fechtner syndrome, Epstein's syndrome, Wiskott-Aldrich syndrome, congenital amegakaryocytic thrombocytopenia, platelet storage pool deficiency, Hermansky-Pudlak syndrome, Bernard-Soulier syndrome, Von Willebrand Disease Type 2B, ANKRD26-related thrombocytopenia, thrombocytopenia absent radius syndrome, familial platelet disorder with associated myeloid malignancy (FPD/AML, associated with mutations in RUNX1), thrombocytopenia associated with a mutation in Filamin-A, or thrombocytopenia associated with a mutation in GATA-1). In some embodiments, the thrombocytopenia is immune thrombocytopenia. In some embodiments, the methods described herein increase platelet levels, increase or induce megakaryocyte differentiation and/or maturation, promote or increase platelet formation or production, reduce the accumulation of platelet progenitor cells, and/or improve blood clotting, reduce bleeding events, and/or reduce bleeding in the skin (petechiae or bruising) compared to measurements obtained prior to treatment or compared to measurements typically observed in untreated subjects having the same disease or condition. In some embodiments, the subject is identified as having thrombocytopenia prior to treatment with a composition described herein. In some embodiments, the method includes a step of identifying the subject as having thrombocytopenia (e.g., by evaluating platelet levels) prior to treatment with a polypeptide described herein. The method can further include evaluating platelet levels after administration of a polypeptide described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing neutropenia by administering to the subject an effective amount of a polypeptide of SEQ ID NO: 1. In any of the methods described herein, a subject having or at risk of developing low neutrophil levels (e.g., low neutrophil cell counts) has or is at risk of developing neutropenia. In some embodiments, the neutropenia is associated with a bone marrow defect, a myelodysplastic syndrome, bone marrow transplantation, myelofibrosis, ineffective hematopoiesis, aplastic anemia, Fanconi anemia, Diamond Blackfan anemia, Shwachman Diamond syndrome, paroxysmal nocturnal hemoglobinuria, Pearson syndrome, dyskeratosis congenita, cancer (e.g., leukemia), a vitamin deficiency (e.g., B-12 deficiency or folate deficiency), an enlarged spleen, an autoimmune disease (e.g., granulomatosis with polyangiitis, lupus (e.g., SLE), Evans syndrome, Felty syndrome, Crohn's disease, or rheumatoid arthritis), a viral infection (e.g., chickenpox, Epstein-Barr, Hepatitis A, Hepatitis B, Hepatitis C, HIV/AIDS, cytomegalovirus, Dengue fever, or measles), a bacterial infection (e.g., tuberculosis, salmonella infection, or sepsis), cancer treatment (e.g., chemotherapy or radiation therapy), treatment with other medications (e.g., a medication used to treat overactive thyroid, such as methimazole and propylthiouracil; an antibiotic, such as vancomycin, penicillin G, trimethoprim, and oxacillin; an antiviral drug, such as ganciclovir and valganciclovir; an anti-inflammatory medication for ulcerative colitis or rheumatoid arthritis, such as sulfasalazine; a drug used to treat irregular heart rhythms, such as quinidine and procainamide; an anticonvulsant, such as phenytoin and valproate; an antipsychotic, such as clozapine; or levamisole), inflammation, hematopoietic stem cell transplantation, or contraindication to transfusion (e.g., patients of advanced age, patients with allo-or auto-antibodies, pediatric patients, patients with cardiopulmonary disease, patients who object to transfusion for religious reasons (e.g., some Jehovah's Witnesses)). The myelodysplastic syndrome may be myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)), myelodysplastic syndrome associated with isolated del chromosome abnormality (MDS with isolated del (5q)), myelodysplastic syndrome with excess blasts (MDS-EB; which includes myelodysplastic syndrome with excess blasts—type 1 (MDS-EB-1) and myelodysplastic syndrome with excess blasts—type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T). The myelodysplastic syndrome may be a very low, low, or intermediate risk MDS as determined by the Revised International Prognostic Scoring System (IPSS-R). The myelodysplastic syndrome may be an RS-positive myelodysplastic syndrome (e.g., the subject with a myelodysplastic syndrome may have ring sideroblasts) or a non-RS myelodysplastic syndrome (e.g., the subject with a myelodysplastic syndrome may lack ring sideroblasts). In some embodiments, the RS-positive myelodysplastic syndrome is associated with a splicing factor mutation, such as a mutation in SF3B1. In some embodiments, the MDS is associated with a defect in terminal maturation (often observed in RS-positive MDS and in subjects having splicing factor mutations). In some embodiments, the MDS is associated with a defect in early-stage hematopoiesis (e.g., commitment or early differentiation). In some embodiments, the MDS is associated with elevated endogenous erythropoietin levels. In some embodiments, the myelodysplastic syndrome is associated with hypocellular bone marrow (e.g., a subject with MDS has hypocellular bone marrow). The subject may have a low transfusion burden or a high transfusion burden. In some embodiments, the subject has a low transfusion burden and received 1-3 RBC units in the eight weeks prior to treatment with a polypeptide described herein. In some embodiments, the subject has a low transfusion burden and did not receive a transfusion (received 0 RBC units) in the eight weeks prior to treatment with a polypeptide described herein. In some embodiments, the subject does not respond well to erythropoietin (EPO) or is susceptible to adverse effects of EPO (e.g., hypertension, headaches, vascular thrombosis, influenza-like syndrome, obstruction of shunts, and myocardial infarction). The compositions and methods described herein can also be used to treat subjects that do not respond to an erythroid maturation agent. In some embodiments, the subject has previously been treated with an ESA. In some embodiments, the subject has not previously been treated with an ESA. In some embodiments, the neutropenia is chronic idiopathic neutropenia. In some embodiments, the neutropenia is familial neutropenia (also referred to as inherited neutropenia, e.g., cyclic neutropenia, chronic benign neutropenia, or severe congenital neutropenia (SCN), which may be associated with mutations in the genes ELANE (associated with SCN1), HAX1 (associated with SCN3), G6PC3 (associated with SCN4), GFI1 (associated with SCN2), CSF3R, WAS (associated with X-linked neutropenia/X-linked SCN), CXCR4, VPS45A (associated with SCN5), or JAGN1). In some embodiments, the methods described herein increase neutrophil levels, increase or induce neutrophil formation or production, and/or increase or induce the differentiation and/or maturation of progenitor cells (e.g., myeloid progenitors, myeloblasts, or myelocytes) into neutrophils compared to measurements obtained prior to treatment or compared to measurements typically observed in untreated subjects having the same disease or condition. In some embodiments, the methods described herein reduce the susceptibility of the subject to infection. In some embodiments, the subject is identified as having neutropenia prior to treatment with a composition described herein. In some embodiments, the method includes a step of identifying the subject as having neutropenia (e.g., by evaluating neutrophil levels) prior to treatment with a polypeptide described herein. The method can further include evaluating neutrophil levels after administration of a polypeptide described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing fibrosis by administering to the subject an effective amount of a polypeptide of SEQ ID NO: 1. In any of the methods described herein, the subject has or is at risk of developing fibrosis. In some embodiments, the fibrosis is fibrosis is chemotherapeutic drug-induced fibrosis, radiation-induced fibrosis, pulmonary fibrosis (e.g., cystic fibrosis, idiopathic fibrosis, or fibrosis related to tuberculosis, pneumonia, or coal dust), hepatic fibrosis (e.g., cirrhosis, biliary atresia), renal fibrosis (e.g., fibrosis related to chronic kidney disease), corneal fibrosis, heart fibrosis (e.g., endomyocardial fibrosis, or fibrosis related to myocardial infarction), bone marrow fibrosis, myelofibrosis, mediastinal fibrosis, retroperitoneal fibrosis, arthrofibrosis, osteoarticular fibrosis, tissue fibrosis (e.g., fibrosis affecting muscle tissue, skin epidermis, skin dermis, tendon, cartilage, pancreatic tissue, uterine tissue, neural tissue, testis, ovary, adrenal gland, artery, vein, bone marrow, colon, small intestine, large intestine, biliary tract, or gut), a tumor stroma, a desmoplastic tumor, a surgical adhesion, a hypertrophic scar, or a keloid. In some embodiments, the fibrosis is associated with a wound, a burn, hepatitis B or C infection, fatty liver disease, Schistosoma infection, kidney disease (e.g., chronic kidney disease), heart disease, macular degeneration, retinal or vitreal retinopathy, Crohn's disease, systemic or local scleroderma, atherosclerosis, or restenosis. In some embodiments, the subject is at risk of developing fibrosis related to cancer treatment (chemotherapy or radiation), disease or infection (e.g., tuberculosis, pneumonia, myocardial infarction, hepatitis B or C infection, fatty liver disease, Schistosoma infection, kidney disease (e.g., chronic kidney disease), heart disease, macular degeneration, retinal or vitreal retinopathy, Crohn's disease, systemic or local scleroderma, atherosclerosis, restenosis, surgery, a wound, or a burn. In some embodiments, the methods described herein reduce fibrosis compared to measurements obtained prior to treatment or compared to fibrosis in untreated subjects. In some embodiments, the methods described herein prevent the development of fibrosis or reduce the risk of developing fibrosis (e.g., reduce the risk of developing fibrosis compared to the development of fibrosis in untreated subjects). In some embodiments, the methods described herein slow or stop the progression of fibrosis (e.g., slow the progression of fibrosis compared to progression prior to treatment or compared to progression without treatment or in an untreated subject). In some embodiments, the methods described herein reduce the frequency or severity of one or more symptom of fibrosis. In some embodiments, the methods described herein improve organ or tissue function (e.g., the function of the organ or tissue having fibrosis) compared to organ or tissue function prior to treatment. Tissue and organ function can be assessed using any standard clinical test commonly used to evaluate tissue and organ function. In some embodiments, the subject is identified as having fibrosis prior to treatment with a composition described herein. In some embodiments, the method includes a step of identifying the subject as having fibrosis (e.g., using imaging to visualize scar formation) prior to treatment with a polypeptide described herein. The method can further include evaluating fibrosis after administration of a polypeptide described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing PH (e.g., PAH, venous PH, hypoxic PH, thromboembolic PH, or miscellaneous PH) by administering to the subject an effective amount of a polypeptide of SEQ ID NO: 1. In any of the methods described herein, the subject may have or be at risk of developing PH. In some embodiments, the PH is PAH (World Health Organization (WHO) Group 1 PH). In some embodiments, the PAH is idiopathic PAH. In some embodiments, the PAH is heritable PAH. In some embodiments, the PAH is PAH related to (e.g., caused by or associated with) HIV infection, schistosomiasis, portal hypertension, pulmonary veno-occlusive disease, pulmonary capillary hemangiomatosis, cirrhosis of the liver, a congenital heart abnormality, a connective tissue/autoimmune disorder (e.g., scleroderma, lupus (systemic lupus erythematosus), mixed connective tissue disease, Sjogren syndrome, an inflammatory idiopathic myopathy, or rheumatoid arthritis), drug use or abuse (e.g., methamphetamine or cocaine use), a toxin, or congenital systemic-pulmonary intracardiac shunt. A subject with PAH associated with congenital systemic-pulmonary intracardiac shunt may have surgical correction or repair with a closure device at least one year prior to treatment initiation and have no, or clinically insignificant, shunt fraction (1.0≤pulmonary-systemic flow ratio≤1.5). In some embodiments, the subject has hemodynamic parameters consistent with a diagnosis of PAH, such as mean pulmonary arterial pressure (mPAP)>20 mm Hg at rest, pulmonary artery wedge pressure (PAWP)≤15 mm Hg, and pulmonary vascular resistance (PVR)≥5 Wood units (400 dyn.sec.cm⁻⁵). In some embodiments, the PH is venous PH (WHO Group 2 PH). In some embodiments, the venous PH is venous PH related to (e.g., caused by or associated with) left ventricular systolic dysfunction, left ventricular diastolic dysfunction, valvular heart disease, congenital cardiomyopathy, or congenital/acquired pulmonary venous stenosis. In some embodiments, the PH is hypoxic PH (WHO Group 3 PH). In some embodiments, the hypoxic PH is hypoxic PH related to (e.g., caused by or associated with) chronic obstructive pulmonary disease (e.g., emphysema), interstitial lung disease, sleep-disordered breathing (e.g., sleep apnea), lung disease (e.g., pulmonary fibrosis), an alveolar hypoventilation disorder, chronic exposure to high altitude, or a developmental abnormality. In some embodiments, the PH is thromboembolic PH (WHO Group 4 PH). In some embodiments, the thromboembolic PH is thromboembolic PH related to (e.g., caused by or associated with) chronic thromboembolic pulmonary hypertension, or other pulmonary artery obstructions (e.g., pulmonary emboli, angiosarcoma, arteritis, congenital pulmonary artery stenosis, or parasitic infection). In some embodiments, the PH is miscellaneous PH (WHO Group 5 PH). In some embodiments, the miscellaneous PH is miscellaneous PH related to (e.g., caused by or associated with) a hematologic disease (e.g., chronic hemolytic anemia, sickle cell disease), a systemic disease (e.g., sarcoidosis, pulmonary Langerhans cell histiocytosis, lymphangioleiomyomatosis, neurofibromatosis, or vasculitis), a metabolic disorder (e.g., glycogen storage disease, Gaucher disease, or a thyroid disease), pulmonary tumoral thrombotic microangiopathy, fibrosing mediastinitis, chronic kidney failure, or segmental pulmonary hypertension. In some embodiments, the subject to be treated according to the methods described herein has WHO/New York Heart Association (NYHA) Functional Class (FC) Il symptoms or FC III symptoms. In some embodiments, the subject to be treated according to the methods described herein has a 6-minute walk distance≥150 and ≤500 meters.

In some embodiments, a subject with PAH treated according to the methods described herein is administered the polypeptide of SEQ ID NO: 1 in combination with one or more PAH background therapies. The one or more PAH background therapies may include an endothelin-receptor antagonist (ERA) (e.g., ambrisentan, bosentan, macitentan, or thelin), a phosphodiesterase-5 inhibitor (PDE5-I) (e.g., e.g., sildenafil, tadalafil, vardenafil), a soluble guanylate cyclase (sGC) stimulator (e.g., riociguat or cinaciguat), a prostacyclin analogue or receptor agonist (e.g., epoprostenol, iloprost, treprostinil, beraprost, or selexipag), an anticoagulant (e.g., warfarin), a diuretic, oxygen therapy, digoxin, a calcium channel blocker (e.g., nifedipine, diltiazem, or amlodipine), atrial septostomy, pulmonary thromboendarterectomy, an ASK-1 inhibitor (e.g., CIIA, SCH79797, GS-4997, MSC2032964A, a 3H-naphtho [1,2,3-de]quiniline-2,7-diones, NQDI-1, 2-thioxo-thiazolidines, or 5-bromo-3-(4-oxo-2-thioxo-thiazolidine-5-ylidene)-1,3-dihydro-indol-2-one), a NF-κB antagonist (e.g., dh404, CDDO-epoxide, 2.2-difluoropropionamide, C28 imidazole (CDDO-Im), 2-cyano-3,12-dioxoolean-1,9-dien-28-oic acid (CDDO), 3-Acetyloleanolic Acid, 3-Triflouroacetyloleanolic Acid, 28-Methyl-3-acetyloleanane, 28-Methyl-3-trifluoroacetyloleanane, 28-Methyloxyoleanolic Acid, SZC014, SCZ015, SZC017, PEGylated derivatives of oleanolic acid, 3-O-(beta-D-glucopyranosyl) oleanolic acid, 3-O-[beta-D-glucopyranosyl-(1→3)-beta-D-glucopyranosyl] oleanolic acid, 3-O-[beta-D-glucopyranosyl-(1→2)-beta-D-glucopyranosyl] oleanolic acid, 3-O-[beta-D-glucopyranosyl-(1→3)-beta-D-glucopyranosyl] oleanolic acid 28-O-beta-D-glucopyranosyl ester, 3-O-[beta-D-glucopyranosyl-(1→2)-beta-D-glucopyranosyl] oleanolic acid 28-O-beta-D-glucopyranosyl ester, 3-O-[a-L-rhamnopyranosyl-(1→3)-beta-D-glucuronopyranosyl] oleanolic acid, 3-O-[alpha-L-rhamnopyranosyl-(1→3)-beta-D-glucuronopyranosyl] oleanolic acid 28-O-beta-D-glucopyranosyl ester, 28-O-β-D-glucopyranosyl-oleanolic acid, 3-O-β-D-glucopyranosyl (1→3)-3-D-glucopyranosiduronic acid (CS1), oleanolic acid 3-O-β-D-glucopyranosyl (1→3)-β-D-glucopyranosiduronic acid (CS2), methyl 3,11-dioxoolean-12-en-28-olate (DIOXOL), ZCVI₄-2, or Benzyl 3-dehydr-oxy-1,2,5-oxadiazolo [3′,4′: 2,3] oleanolate), or lung and/or heart transplantation. In some embodiments, the subject is treated with a single PAH background therapy (e.g., an ERA, PDE5-I, sGC stimulator, or prostacyclin analogue or receptor agonist). In some embodiments, the subject is treated with at least two PAH background therapies (e.g., two of an ERA, PDE5-I, sGC stimulator, or prostacyclin analogue or receptor agonist). For example, a subject may be treated with an ERA and a PDE5-I, an ERA and an sGC stimulator, an ERA and a prostacyclin receptor analogue or agonist, a PDE5-I and a prostacyclin receptor analogue or agonist, an sGC stimulator, and a prostacyclin receptor analogue or agonist, an ERA, a PDE5-I, and a prostacyclin receptor analogue or agonist, or an ERA, an sGC stimulator, and a prostacyclin receptor analogue or agonist. The subject is not to be treated with both a PDE5-I and an sGC stimulator. In some embodiments, the subject has been taking the one or more PAH background therapies prior to treatment with the polypeptide of SEQ ID NO: 1 (e.g., taking the one or more PAH background therapies for 1 week, 1 month, 2 months, 3 months, 4 months, 5 months, 6 months, 1 year, 2 years, or longer prior to treatment initiation with the polypeptide of SEQ ID NO: 1). In some embodiments, the subject has been taking the one or more PAH background therapies for at least 90 days prior to treatment initiation with the polypeptide of SEQ ID NO: 1. In some embodiments, the subject has been on stable PAH background therapy for at least 90 days prior to treatment initiation with the polypeptide of SEQ ID NO: 1 (stable therapy is defined as no change in dose/regimen of PAH background therapy). In some embodiments, the PAH background therapy is administered as indicated on the label (e.g., for the treatment of subjects with PAH).

In some embodiments, the methods described herein reduce the symptoms (e.g., reduce the severity or frequency of symptoms, such as shortness of breath (dyspnea), fatigue, swelling (e.g., edema) of the legs, feet, belly (ascites), or neck, chest pain or pressure, racing pulse or heart palpitations, bluish color to lips or skin (cyanosis), dizziness, or fainting) of PH compared to the frequency or severity of symptoms prior to treatment. In some embodiments, the methods described herein prevent the development of PH or reduce the risk of developing PH (e.g., reduce the risk of developing PH compared to the development of PH in untreated subjects). In some embodiments, the methods described herein slow or stop the progression of PH (e.g., slow the progression of PH compared to progression prior to treatment or compared to progression without treatment or in an untreated subject). In some embodiments, the methods described herein reduce pulmonary vascular remodeling or vascular remodeling in the heart of a subject (e.g., the initiation or progression of vascular remodeling in the heart or lungs) compared to vascular remodeling prior to treatment or compared to vascular remodeling in an untreated subject. In some embodiments, the methods described herein reduce right ventricular hypertrophy (e.g., reduce right ventricular hypertrophy or the progression of right ventricular hypertrophy) compared to right ventricular hypertrophy prior to treatment or compared to right ventricular hypertrophy in an untreated subject. In some embodiments, the methods described herein reduce PH-associated bone loss (e.g., reduce PAH-associated bone loss, such as preventing or reducing the reduction in bone mineral density that occurs in subjects with PAH) compared to bone loss prior to treatment or compared to bone loss in an untreated subject. In some embodiments, the methods described herein reduce pulmonary arterial muscularization and/or pulmonary arterial wall thickening compared to pulmonary arterial muscularization and/or pulmonary arterial wall thickening prior to treatment or compared to pulmonary arterial muscularization and/or pulmonary arterial wall thickening in an untreated subject. In some embodiments, the methods described herein reduce right ventricular compensation compared to right ventricular compensation prior to treatment or compared to right ventricular compensation in an untreated subject. Symptoms of PH can be evaluated before and after treatment using standard clinical tests. Commonly used tests for evaluating PH include electrocardiograms, pulmonary function tests, echocardiograms, right heart catheterization, computed tomography scan, measurement of pulmonary vascular resistance, and the 6-minute walk test. In some embodiments, the methods described herein reduce pulmonary vascular resistance (e.g., result in a reduction in pulmonary vascular resistance compared to pulmonary vascular resistance prior to treatment). In some embodiments, the methods described herein improve performance in the 6-minute walk test (i.e., increase 6-minute walk distance) compared to performance in the 6-minute walk test prior to treatment. In some embodiments, the methods described herein improve WHO/NYHA FC compared to baseline pre-treatment assessments. In some embodiments, the methods described herein lead to improvements in one or more of mPAP, cardiac output (CO), cardiac index (CI), PAWP, right atrial pressure (RAP), mixed venous oxygen saturation (SvO2), stroke volume (SV), stroke volume index (SVI), and pulmonary artery compliance (PAC) compared to baseline pre-treatment measurements. In some embodiments, the methods described herein lead to a change (e.g., decrease) in NT-proBNP from baseline pre-treatment measurements. In some embodiments, the methods described herein attenuate clinical worsening (e.g., reduce the incidence of clinical worsening or increase the time to clinical worsening, e.g., the incidence or time to first clinical worsening). In some embodiments, the methods described herein lead to an improvement in risk stratification measures (e.g., lead to an improvement or a maintenance of low risk in ESC/ERC 4-strata risk category assessment or lead to an improvement in REVEAL (Registry to Evaluate Early and Long-term PAH Disease Management) Lite 2 and/or COMPERA 2.0) as compared to measures prior to treatment initiation. In some embodiments, the methods described herein improve physical activity (overall activity) compared to baseline pre-treatment measurements (e.g., as measured by actigraphy). In some embodiments, the methods described herein improve health-related quality of life (HRQOL), which can be assessed as a change from baseline HRQOL measures using Pulmonary Arterial Hypertension-Symptoms and Impact (PAH-SYMPACT) and/or emPHasis-10 assessments. In some embodiments, the methods described herein lead to changes in biomarkers such as BSAP, connective tissue growth factor (CTGF), galectin, and osteopontin compared to pre-treatment measurements. The aforementioned effects may be observed after 1, 2, 3, 4, 5, 6, 7, 8, 9, 10, 12, 16, 20, 24, 28, 32, 36, 40, 44, 48, 52, 56, 60, 64, 68, 72, 76, 80, 84, 88, 92, 96, 100 or more weeks of treatment with the polypeptide of SEQ ID NO: 1. In some embodiments, the subject is identified as having PH prior to treatment with a polypeptide described herein. In some embodiments, the method includes a step of identifying the subject as having

PH (e.g., by evaluating symptoms of PH) prior to treatment with a polypeptide described herein. The method can further include evaluating PH symptoms after administration of a composition described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

The invention also includes methods of treating a subject having or at risk of developing a metabolic disease (e.g., obesity, Type 1 diabetes, or Type 2 diabetes) by administering to the subject an effective amount of a polypeptide of SEQ ID NO: 1. In some embodiments, the subject may have a disease that results in obesity. In some embodiments, the polypeptide of SEQ ID NO: 1 may be administered to a subject to prevent the development of obesity (e.g., in a subject at risk of developing obesity, e.g., a subject who is overweight, who has a family history of obesity, or who has other medical conditions or risk factors linked to increased risk of obesity (e.g., advanced age, or treatment with a medication associated with the development of obesity, such as a glucocorticoid (e.g., a corticosteroid, such as prednisone), a selective serotonin reuptake inhibitors (SSRI, e.g., paroxetine, mirtazapine, fluoxetine, escitalopram, sertraline), a tricyclic antidepressant (e.g., amitriptyline), a mood stabilizer (e.g., valproic acid, lithium), an antipsychotic (e.g., olanzapine, chlorpromazine, clozapine), and a diabetes medication (e.g., insulin, chlorpropamide)) and/or to treat a subject already diagnosed with obesity. In some embodiments, the subject has age-related obesity or metabolic disease. In some embodiments, the subject has treatment-related obesity or metabolic disease. Administration of a composition described herein may reduce bodyweight by decreasing the amount of body fat. In some embodiments, the composition decreases the amount of body fat while maintaining or increasing the amount of lean mass.

In some embodiments, the polypeptide described herein may be administered to a subject to prevent the development of diabetes (e.g., Type 1 or Type 2 diabetes, e.g., in a subject at risk of developing diabetes associated with advanced age or treatment with a medication associated with the development of diabetes, such as a glucocorticoid (e.g., a corticosteroid, e.g., glucocorticoid-induced diabetes mellitus), an SSRI, a serotonin-norepinephrine reuptake inhibitors (SNRI), a mood stabilizer (e.g., lithium and valproic acid), and an antipsychotic (e.g., olanzapine and clozapine)) and/or to treat a subject already diagnosed with diabetes. In some embodiments, the subject has age-related diabetes or metabolic disease. In some embodiments, the subject has treatment-related diabetes or metabolic disease. Subjects who are likely to develop diabetes, e.g., subjects with a genetic predisposition to diabetes, a family history of diabetes, prediabetes, an autoimmune disease associated with diabetes, another metabolic disease, subjects of advanced age, or subjects treated with a medication associated with the development of diabetes may be administered the polypeptide of SEQ ID NO: 1 prophylactically, such that the polypeptide may maintain the normal function and health of β-cells and/or prevent or delay autoimmune inflammatory damage to B-cells. In other embodiments, the polypeptide of SEQ ID NO: 1may be administered to individuals before diagnosis with diabetes (e.g., Type 1 and Type 2 diabetes) or the development of clinical symptoms of diabetes, e.g., high blood glucose level, high fasting insulin level, insulin resistance, polyuria, polydipsia, and polyphagia. In some embodiments, the polypeptide may be administered to patients prior to the patient needing insulin. In some embodiments, the administration of the polypeptide may delay, reduce, or eliminate the need for insulin treatment in diabetic patients. For example, administration of the polypeptide described herein to a subject may help to increase the rate of glucose clearance from the blood.

In some embodiments, the methods described herein reduce body fat (e.g., reduce the amount of subcutaneous, visceral, and/or hepatic fat, reduce adiposity, reduce the weights of epididymal and perirenal fat pads, or reduce body fat percentage). In some embodiments, the methods described herein reduce body weight or reduce body weight gain (e.g., reduce the percentage of body weight gain). In some embodiments, the methods described herein reduce the proliferation of adipose cells. In some embodiments, the methods described herein reduce LDL. In some embodiments, the methods described herein reduce triglycerides. In some embodiments, the methods described herein improve the serum lipid profile of the subject. In some embodiments, the methods described herein reduce body fat and increase muscle mass. In some embodiments, the methods described herein reduce blood glucose levels (e.g., fasting glucose levels) or and/or increase glucose clearance. In some embodiments, the methods described herein reduce fasting insulin levels and/or improve insulin sensitivity (e.g., reduce insulin resistance). In some embodiments, the methods described herein regulate insulin biosynthesis and/or secretion from B-cells. These outcomes can be assessed by comparing measurements obtained after treatment to measurements taken prior to treatment. In some embodiments, the methods described herein do not affect the appetite for food intake. The polypeptide of SEQ ID NO: 1 may decrease body fat, decrease body weight, or increase insulin sensitivity and/or glucose clearance by increasing muscle mass. In some embodiments, the subject is identified as having a metabolic disease prior to treatment with a composition described herein. In some embodiments, the method includes a step of identifying the subject as having a metabolic disease (e.g., by evaluating body weight, body fat, glucose clearance, or insulin sensitivity) prior to treatment with a polypeptide described herein. The method can further include evaluating body fat (e.g., amount of body fat or body fat percentage), body weight or body weight gain, fasting insulin levels, glucose clearance, serum lipid profile, or insulin sensitivity after administration of a polypeptide described herein (e.g., 12 hours, 24 hours, 1, 2, 3, 4, 5, 6, or 7 days, 1, 2, 3, 4, 5, 6, 7, or 8 weeks, or 1, 2, 3, 4, 5, or 6 months or more after treatment initiation).

In some embodiments, the polypeptide of SEQ ID NO: 1 can be administered to increase EPO levels (e.g., serum EPO levels) and/or EPO receptor levels (e.g., EPO receptor levels in bone marrow cells) in a subject in need thereof (e.g., a subject with low serum EPO). The invention also includes methods of treating a subject having or at risk of developing (e.g., treating, delaying the development of, and/or preventing) a disease or condition that can be treated with EPO or an ESA (e.g., a disease or condition that can be treated by increasing EPO or EPO receptor levels) by administering to the subject an effective amount of a polypeptide of SEQ ID NO: 1. Diseases and conditions that can be treated by increasing EPO or EPO receptor levels include end-stage renal disease, renal insufficiency, polycythemia, iron overload (e.g., hemochromatosis), pregnancy, a menstrual disorder, space flight, ischemia (CNS ischemia, liver ischemia, renal ischemia, or cardiac ischemia), ulcers, burns, wounds (e.g., chronic wounds), ischemia-reperfusion injury (e.g., ischemia-reperfusion injury associated with surgery or organ transplantation), an ischemic disorder or condition (e.g., myocardial infarction, ischemic stroke, occlusive arterial disease, chronic venous insufficiency, pulmonary embolism, circulatory shock, such as hemorrhagic, septic, or cardiogenic shock, acute respiratory failure, chronic heart failure, atherosclerosis, cardiac cirrhosis, macular degeneration, sleep apnea, Raynaud's disease, systemic sclerosis, nonbacterial thrombotic endocarditis, angina pectoris, transient ischemic attacks, chronic alcoholic liver disease, or ischemia resulting from general anesthesia), hypoxia (e.g., perinatal hypoxia or a hypoxic condition or disorder such as a pulmonary disorder (e.g., hypoxic hypoxia, such as COPD), severe pneumonia, pulmonary edema, hyaline membrane disease, liver or renal disease, cancer or other chronic illness, and altitude sickness), and aging. A polypeptide of SEQ ID NO: 1 can also be used to treat a subject receiving kidney dialysis, to treat a subject who has recently received a stem cell transplant, or as a pretreatment or further treatment for a tissue or organ to be transplanted (such as for treatment of the tissue or organ before (e.g., directly before), during, or directly after transplantation). Given that EPO has been found to stimulate the mobilization, proliferation, migration, and differentiation of endothelial progenitor cells, the polypeptide of SEQ ID NO: 1 can also be used to treat a disease associated with dysfunction of endothelial progenitor cells. Such diseases include heart failure, angina pectoris, endotheliosis (e.g., reticuloendotheliosis), age-related cardiovascular disorder, coronary heart disease, atherosclerosis, myocardial ischemia, hypercholesterolemia, ischemic disorders of the extremities, Raynaud's disease, preeclampsia, pregnancy-induced hypertension, endothelium-mediated chronic inflammatory disorders (e.g., inflammation of the vessels), wound healing, and chronic or acute renal failure (also referred to as chronic kidney disease and acute kidney failure, respectively). Since EPO has been shown to have a mitogenic and chemotactic effect on vascular endothelial cells, the polypeptide of SEQ ID NO: 1 can also be used to promote the growth of new blood vessels (vasculogenesis) and/or the replacement of damaged vascular regions through local formation of new blood vessels, such as collateral coronary blood vessels (e.g., those that may occur after myocardial infarction), for granulation tissue formation (e.g. in damaged tissue, wounds, and ulcers), for trauma treatment, for post-vascular graft treatment, and for production of vascular prostheses such as heart valves.
EPO has also been found to have anti-inflammatory and neuroprotective effects. Therefore, the polypeptide of SEQ ID NO: 1 can also be used to treat a neurological disorder and/or an inflammatory brain disease, such as a demyelinating disease (e.g., multiple sclerosis, neuromyelitis optica, acute disseminated encephalomyelitis, transverse myelitis), epilepsy, spinal cord injury (e.g., an acute spinal cord injury), a complication following traumatic brain injury (e.g., to treat a symptom of the traumatic brain injury, such as hypotension, hypoxemia, brain swelling, headache, neck pain, difficulty remembering, difficulty concentrating, difficulty making decisions, fatigue, a mood change, nausea, photophobia, blurred vision, ear ringing, a loss of sense of taste, a loss of sense of smell, a seizure, coma, muscle weakness, paralysis, or a progressive decline in neurologic function), a chronic inflammatory brain disease (e.g., a neurodegenerative disease, such as Alzheimer's disease (AD), Parkinson's disease (PD), Huntington's disease, amyotrophic lateral sclerosis (ALS), or age-related macular degeneration (AMD)), or a neurological disorder associated with surgery, such as thoracoabdominal aortic surgery, in addition to diseases or conditions that have an inflammatory or autoimmune component, such as acute cerebrovascular injury, acute brain injury, acute cardiovascular injury, arthritis, an autoimmune disease, a stroke, a neurological injury, and immune-mediated inflammation. The polypeptide may treat the neurological disorder or inflammatory brain disease by reducing infiltration of mononuclear cells into the brain of the subject, improving a neurological deficit, and/or reducing axonal damage and/or neuronal and/or glial cell death in at least one region of the brain of the subject affected, directly or indirectly, by the disease, disorder, or condition.

Gastrointestinal dysmotility can also be treated using EPO. Accordingly, the polypeptide of SEQ ID NO: 1 may be used to treat gastrointestinal dysmotility due to intestinal injury, abdominal trauma, an intestinal inflammatory condition (e.g., an inflammatory bowel disease (IBD), such as Crohn's Disease and Ulcerative Colitis), an intestinal infection (e.g., a bacterial infection, such as an infection that leads to sepsis and bacteremia and localized infections such as peritonitis and ascites), slow transit constipation (e.g., chronic constipation, idiopathic constipation, constipation due to post-operative ileus, or constipation caused by opiate use), post-operative ileus, a neurodegenerative injury, a neurotraumatic injury, a congenital problem (e.g., Gastroschisis, omphalocele, aganglionic megacolon, Hirschsprung's disease, chronic intestinal pseudo-obstruction, small left colon syndrome, an anorectal anomaly, esophageal dysplasia and atresias, ectopic anus, a congenital hernia, internal anal sphincter achalasia), or a malnutrition-malabsorption problem (e.g., due to an intestinal injury, an abdominal trauma, an intestinal inflammatory condition, an intestinal infection, constipation (e.g., constipation caused by opiate use), post-operative ileus, a neurodegenerative injury, a neurotraumatic injury, a congenital problem, Gaucher disease, refeeding syndrome, extremely low birth weight infants, cancer cachexia, infection, cancer, spinal cord dysfunction, spinal dysraphism, bifida, tumor, central nervous system dysfunction, peripheral neuropathy, removal of part of the gastrointestinal tract, hemorrhage, liver dysfunction, celiac disease, cystic fibrosis, a muscular dystrophy, or cerebral palsy).

The polypeptide of SEQ ID NO: 1 can also be used to treat chronic or recurrent disease such as asthma, a viral disease or infection (e.g., HIV infection or HCV infection), hypertension, a systemic microbial infection, cancer, a disease of the endocrine system, a disease of the reproductive system, psychosis, a genetic disease, allergy, a gastrointestinal disease, arterial sclerosis, a cardiovascular disease, graft-vs-host disease, or an inflammatory disease. The polypeptide of SEQ ID NO: 1 can also be used to enhance athletic performance, improve exercise capacity, and facilitate or enhance aerobic conditioning. Such methods can be used, e.g., by athletes to facilitate training and by soldiers to improve stamina and endurance. In some embodiments, the methods described herein are directed to affecting myostatin, activin A, activin B, and/or GDF-11 signaling (e.g., reducing or inhibiting the binding of activin A, activin B, myostatin, and/or GDF-11 to their endogenous receptors, e.g., ActRIIA and/or ActRIIB) in a subject having a disease or condition that can be treated with EPO or an ESA. In some embodiments, the methods described herein increase EPO levels (e.g., serum EPO levels) and/or EPO receptor levels (e.g., bone marrow EPO receptor levels) compared to measurements obtained prior to treatment or compared to measurements obtained from untreated subjects or control treated subjects having the same disease or condition.

In any of the methods described herein, a dimer (e.g., homodimer) formed by the interaction of Fc domain monomers in SEQ ID NO: 1 may be used as the therapeutic protein. Nucleic acids encoding the polypeptide described herein, or vectors containing said nucleic acids can also be administered according to any of the methods described herein. In any of the methods described herein, the polypeptide, nucleic acid, or vector can be administered as part of a pharmaceutical composition.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1

### Treatment of Human Subjects With Multiple Doses of ActRIIB 2.12-Fc

Healthy postmenopausal women were enrolled in a randomized, double-blind, placebo-controlled, two-part study to assess the safety, tolerability, and pharmacokinetics ActRIIB 2.12-Fc (a homodimer of the polypeptide of SEQ ID NO: 1). Inclusion criteria included being between 45 and 70 years of age, serum FSH>40 IU/L, and a BMI>18.5 kg/m² to <32.0 kg/m², and exclusion criteria included a history of or past treatment for osteoporosis and systemic hormone replacement therapy within three months of the study. In Part 1, participants received either placebo or a single subcutaneous dose of ActRIIB 2.12-Fc at a dose of 0.75 mg/kg, 1.5 mg/kg, 3 mg/kg, or 5.0 mg/kg, and in Part 2, participants received three doses of either placebo or 0.75 mg/kg, 1.5 mg/kg, or 4.5 mg/kg of ActRIIB 2.12-Fc administered subcutaneously once every 28 days over a 12-week period with a 16-week safety follow-up. In addition to safety, tolerability, and pharmacokinetics, pharmacodynamic endpoints, such as biomarkers of bone formation and resorption, were also assessed. Demographics for patients enrolled in Part 1 and Part 2 of the study are provided in Table 1 and Table 2 below.

TABLE 1
Demographics and Disposition (Part 1 SAD)

| | | | | | | All |
|---|---|---|---|---|---|---|
| | PBO | 0.75 mg/kg | 1.5 mg/kg | 3.0 mg/kg | 5.0 mg/kg | Subjects |
| | (N = 8) | (N = 8) | (N = 8) | (N = 8) | (N = 8) | (N = 40) |
| Age, years | | | | | | |
| mean (range) | 56.0 | 58.3 | 54.9 | 57.8 | 59.3 | 57.2 |
| | (48-60) | (52-70) | (50-59) | (50-66) | (53-68) | (48-70) |
| Race, n (%) | | | | | | |
| White | 8 | 8 | 8 | 7 | 8 | 39 |
| | (100) | (100) | (100) | (87.5) | (100) | (97.5) |
| Multiple^{&} | 0 | 0 | 0 | 1 | 0 | 1 |
| | | | | (12.5) | | (2.5) |
| Weight, kg | | | | | | |
| mean (SD) | 68.4 | 71.6 | 67.5 | 68.1 | 67.1 | 68.6 |
| | (10.09) | (9.60) | (8.05) | (9.49) | (10.35) | (9.19) |
| FSH, IU/L | | | | | | |
| mean (SD) | | | | | | |
| [range] | | | | | | |
| at Screening | 88.9 (16.34) | 75.5 (19.87) | 95.0 (22.93) | 77.9 (26.31) | 91.0 (35.02) | 85.6 (25.02) |
| | [62, 107] | [56, 112] | [64, 133] | [60, 127] | [45, 146] | [45, 146] |
| at C 1 D 1 | 70.4 (28.91) | 53.3 (28.16) | 86.5 (16.64) | 49.5 (23.65) | 87.1 (35.49) | 68.9 (30.18) |
| | [18, 105] | [26, 103] | [64, 109] | [21, 92] | [63, 162] | [18, 162] |
| % chg from | −16.9 (35.65) | −31.9 (23.02) | −7.7 (8.78) | −33.3 (24.57) | 4.4 (17.71) | −17.7 (26.38) |
| SCRN | [−83.2, 11.9] | [−58.3, 1.1] | [−18.3, 7.1] | [−83.5, 2.6] | [−17.0, 40.0] | [−83.5, 40.0] |
| Disposition | | | | | | |
| Completed | 8 | 8 | 7 | 8 | 8 | 39 |
| Study, n (%) | (100%) | (100%) | (87.5%) | (100%) | (100%) | (97.5%) |
| Discontinuation, | 0 | 0 | 1^{#} | 0 | 0 | 1^{#} |
| n (%) | | | (12.5%) | | | (2.5) |

^{&}More than one race was reported.
^{#}1 subject prematurely discontinued after receiving ActRIIB 2.12-Fc due to withdrawal of consent.

TABLE 2
Demographics and Disposition (Part 2 MAD)

| | PBO | 0.75 mg/kg | 1.5 mg/kg | 4.5 mg/kg | All Subjects |
|---|---|---|---|---|---|
| | (N = 6) | (N = 8) | (N = 6) | (N = 6) | (N = 26) |
| Age, years | | | | | |
| mean (range) | 59.5 | 59.1 | 55.7 | 61.2 | 58.9 |
| | (51-68) | (52-65) | (52-59) | (52-71) | (51-71) |
| Race, n (%) | | | | | |
| White | 6 | 7 | 5 | 6 | 24 |
| | (100.0) | (87.5) | (83.3) | (100.0) | (92.3) |
| Asian | 0 | 1 | 0 | 0 | 1 |
| | | (12.5) | | | (3.8) |
| Australian Aborigine or | 0 | 0 | 1 | 0 | 1 |
| Torres Strait Islander | | | (16.7) | | (3.8) |
| Weight, kg | | | | | |
| mean (SD) | 68.2 | 67.8 | 71.0 | 71.9 | 69.6 |
| | (12.20) | (7.02) | (9.05) | (12.08) | (9.61) |
| FSH, IU/L | | | | | |
| mean (SD) [range] | | | | | |
| at Screening | 80.7 (17.50) | 69.3 (40.52) | 82.7 (32.01) | 92.5 (32.65) | 80.3 (31.86) |
| | [59, 108] | [41, 164] | [42, 121] | [48, 137] | [41, 164] |
| at C 1 D 1 | 82.8 (20.29) | 65.4 (28.75) | 80.7 (23.65) | 83.8 (21.40) | 77.2 (24.15) |
| | [51, 114] | [40, 126] | [52, 116] | [61, 119] | [40, 126] |
| % chg from SCRN | 5.8 (34.66) | −1.5 (12.94) | 1.7 (14.34) | −4.5 (23.81) | 0.2 (21.35) |
| | [−26, 73] | [−23, 12] | [−19, 24] | [−19, 44] | [−26, 73] |
| Disposition | | | | | |
| Completed Study, n (%) | 5 | 8 | 5 | 6 | 24 |
| | (83.3) | (100.0) | (83.3) | (100.0) | (92.3) |
| Discontinuation, n (%) | 1 | 0 | 1 | 0 | 2 |
| | (16.7)^{&} | | (16.7)^{#} | | (7.7)^{&#} |

^{&}1 subject prematurely discontinued after receiving 2 doses of placebo due to physician's decision
^{#}1 subject withdrew consent after receiving 2 doses of ActRIIB 2.12-Fc

ActRIIB 2.12-Fc was generally well tolerated after repeated doses between 0.75 mg/kg to 4.5 5 mg/kg. The adverse events observed were typical, and there were no clinically relevant trends in vital signs, ECG, or laboratory assessments. One SAE was reported (breast cancer in PBO arm), but other adverse events were generally mild in nature and most resolved during the course of the study. There were no discontinuations due to treatment-related adverse events. This is summarized in Table 3, below. Data are shown as count and (percent) of participants reporting AE.

TABLE 3
Adverse Events Observed During Study

| | Part 1: Single Ascending Dose | | | | | Part 2: Multiple Ascending Doses | | | |
|---|---|---|---|---|---|---|---|---|---|
| | | Dose of ActRIIB 2.12-Fc (mg/kg) | | | | | Dose of ActRIIB 2.12-Fc (mg/kg) | | |
| Adverse | PBO | 0.75 | 1.5 | 3.0 | 5.0 | PBO | 0.75 | 1.5 | 4.5 |
| Event | (N = 8) | (N = 8) | (N = 8) | (N = 8) | (N = 8) | (N = 6) | (N = 8) | (N = 6) | (N = 6) |
| Any TEAE | 6 | 7 | 3 | 6 | 3 | 6 | 5 | 5 | 6 |
| | (75%) | (87.5%) | (37.5%) | (75%) | (37.5%) | (100%) | (62.5%) | (83.3%) | (100%) |
| Any SAE | — | — | — | — | — | 1 | — | — | — |
| | | | | | | (16.7%) | | | |
| Injection | — | 1 | — | 2 | — | — | 2 | 3 | 4 |
| site | | (12.0%) | | (25.0%) | | | (25.0%) | (50.0%) | (66.7%) |
| erythema | | | | | | | | | |
| Headache | 1 | 2 | — | — | 1 | 2 | 2 | 1 | 2 |
| | (12.0%) | (25.0%) | | | (12.5%) | (33.3%) | (25.0%) | (16.7%) | (33.3%) |
| Back pain | 2 | — | 1 | 3 | — | — | — | 1 | — |
| | (25.0%) | | (12.5%) | (37.5%) | | | | (16.7%) | |
| COVID-19 | — | — | — | — | 1 | — | — | 1 | 1 |
| | | | | | (12.5%) | | | (16.7%) | (16.7%) |
| Diarrhoea | 1 | — | — | 1 | 1 | — | — | — | — |
| | (12.0%) | | | (12.5%) | (12.5%) | | | | |
| Pain in | — | — | — | 1 | — | 1 | — | — | 1 |
| extremity | | | | (12.5%) | | (16.0%) | | | (16.7%) |

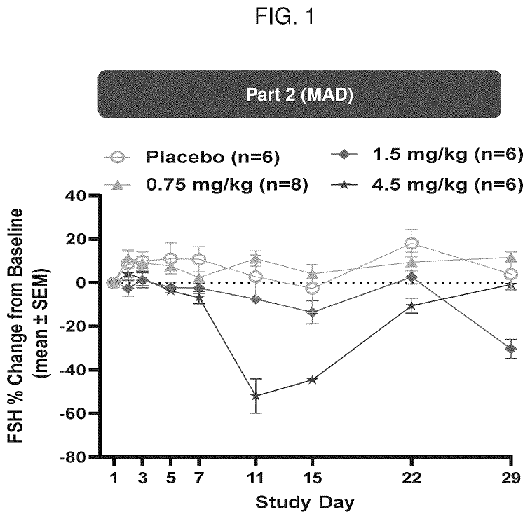

Follicle stimulating hormone (FSH) was measured to assess activin target engagement. FSH secretion by the pituitary is controlled through signaling by the activin receptor and Gonadotropin Releasing Hormone (GnRH), with approximately 50% of FSH secretion regulated via activin signaling and the other 50% by GnRH. Complete inhibition of activin signaling would be expected to reduce FSH by ˜50% in postmenopausal women, who have elevated FSH levels. Treatment with ActRIIB 2.12-Fc resulted in suppression of FSH and dose-dependent reductions were observed. In Part 2, maximal target engagement was observed in the 4.5 mg/kg dose cohort, with a mean (standard deviation, “SD”) 52.0 (19.32) % reduction in FSH. Five out of six subjects who received a 4.5 mg/kg dose of ActRIIB 2.12-Fc achieved a ≥40% reduction in serum FSH levels from baseline (FIG. 1). The magnitude of FSH reduction at the highest doses tested suggests that treatment with ActRIIB 2.12-Fc maximally inhibited activin signaling.

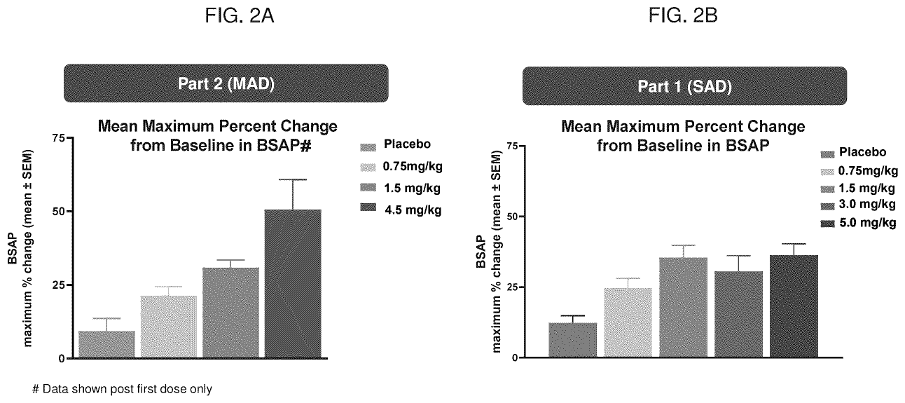

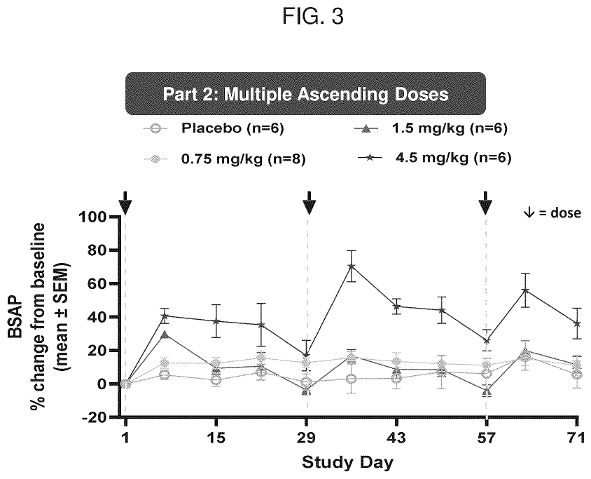

Serum bone-specific alkaline phosphatase (BSAP) was also assessed as a highly specific biomarker of osteoblast activity and as an indicator of activin inhibition and enhanced BMP signaling. Dose-dependent increases in serum levels of BSAP were observed starting at the lowest dose of 0.75 mg/kg. The highest increase in BSAP in Part 2 was observed in the 4.5 mg/kg dose cohort, with mean (SD) maximum increases from baseline of 76.5 (20.33) % (FIG. 2A). BSAP data from Part 1 are shown in FIG. 2B. Further, increases in BSAP were observed after each dose of ActRIIB 2.12-Fc in Part 2 when administered at 28-day intervals (black arrows on graph in FIG. 3), which is supportive of activation of osteoblasts after each dose potentially due to increased bone morphogenic protein signaling.

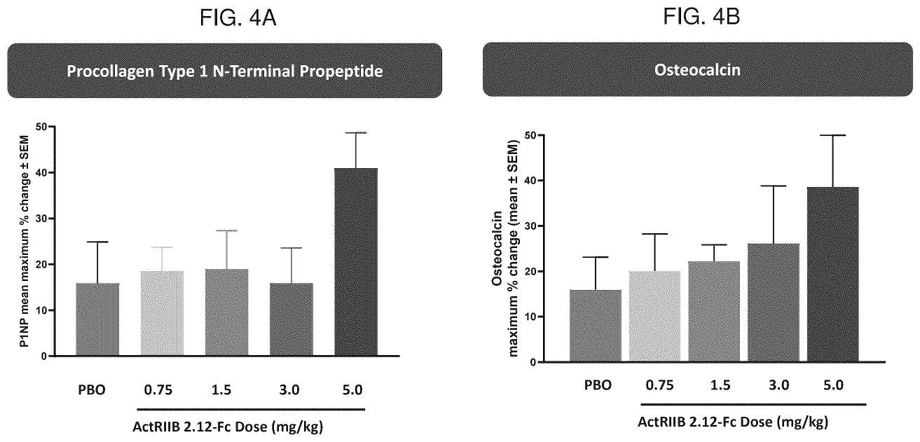

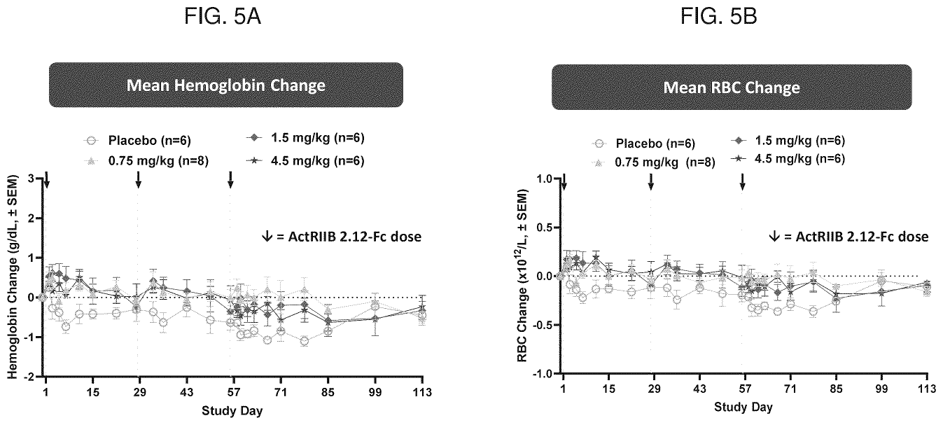

Serum Procollagen Type 1 N-Terminal Propeptide (P1NP) and Osteocalcin, additional markers of bone formation, were assessed in Part 1 of the study. Osteocalcin is a marker of late osteoblastic activity and PN1P is a marker of osteoblast activity and new bone formation. Results of these measurements after a single dose of ActRIIB 2.12-Fc are shown for P1NP in FIG. 4A and for osteocalcin in FIG. 4B. Hemoglobin and red blood cells (RBCs) were also monitored during the study. No clinically meaningful changes in hemoglobin or RBCs were observed in any of the multiple-dose cohorts after treatment with three doses of ActRIIB 2.12-Fc at 28-day intervals for the duration of the study (FIGS. 5A-5B).

In summary, ActRIIB 2.12-Fc was generally well tolerated at multiple doses up to 4.5 mg/kg and adverse events were generally mild. No clinically meaningful changes in hemoglobin or RBCs were observed. The observed reduction in FSH is suggestive of maximum activin target engagement and robust changes in BSAP were observed, starting at the lowest dose (0.75 mg/kg) and maximized at the highest dose administered in Part 2 of the study (4.5 mg/kg).

### Example 2

### Treatment of a Muscle Disease by Administration of a Polypeptide of SEQ ID NO: 1

According to the methods disclosed herein, a physician of skill in the art can treat a subject, such as a human patient, having a muscle disease (e.g., a neuromuscular disease, such as a muscular dystrophy, IBM, SMA, CMT, ALS, myasthenia gravis, or multiple sclerosis; sarcopenia; or cachexia) so as to increase muscle mass or maintain or improve muscle strength (e.g., reduce muscle weakness). The method of treatment can include diagnosing or identifying a subject as a candidate for treatment based on standard clinical tests for muscle diseases (e.g., blood test, muscle biopsy, genetic test, and/or electromyogram). To treat the subject, a physician of skill in the art can administer to the subject a composition containing a polypeptide of SEQ ID NO: 1. The composition containing the polypeptide may be administered to the subject, for example, by subcutaneous injection or by local administration (e.g., injection into the muscle) to treat muscle disease. The polypeptide of SEQ ID NO: 1 is administered in an amount of from 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) once every 28 days. The polypeptide of SEQ ID NO: 1 is administered in an amount sufficient to increase muscle mass or maintain or improve muscle strength (e.g., reduce muscle weakness).

Following administration of the composition to a patient, a practitioner of skill in the art can monitor the patient's improvement in response to the therapy by a variety of methods. For example, a physician can monitor the patient's muscle mass, muscle strength, and motor function. A finding that the patient exhibits increased muscle mass or maintains or improves muscle strength following administration of the composition compared to test results prior to administration of the composition indicates that the patient is responding favorably to the treatment. Subsequent doses can be determined and administered as needed.

### Example 3

### Treatment of a Bone Disease by Administration of a Polypeptide of SEQ ID NO: 1

According to the methods disclosed herein, a physician of skill in the art can treat a subject, such as a human patient, having a bone disease (e.g., osteoporosis, osteogenesis imperfecta, or osteopenia) so as to increase bone mineral density, increase bone formation, reduce bone resorption, reduce bone loss, or reduce the risk or occurrence of bone fracture. The method of treatment can include diagnosing or identifying a subject as a candidate for treatment based on standard clinical tests for bone mineral density (e.g., dual X-ray absorptiometry). To treat the subject, a physician of skill in the art can administer to the subject a composition containing a polypeptide of SEQ ID NO: 1. The composition containing the polypeptide may be administered to the subject, for example, by subcutaneous injection to treat bone disease. The polypeptide of SEQ ID NO: 1 is administered in an amount of from 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) once every 28 days. The polypeptide of SEQ ID NO: 1 is administered in an amount sufficient to increase bone mineral density, increase bone formation, reduce bone resorption, reduce bone loss, or reduce the risk or occurrence of bone fracture.

Following administration of the composition to a patient, a practitioner of skill in the art can monitor the patient's improvement in response to the therapy by a variety of methods. For example, a physician can monitor the patient's bone mineral density by performing dual X-ray absorptiometry. A finding that the patient exhibits increased bone mineral density, increased bone formation, reduced bone resorption, reduced bone loss, or a reduced risk or occurrence of bone fracture following administration of the composition compared to test results prior to administration of the composition indicates that the patient is responding favorably to the treatment. Subsequent doses can be determined and administered as needed.

### Example 4

### Treatment of Fibrosis by Administration of a Polypeptide of SEQ ID NO: 1

According to the methods disclosed herein, a physician of skill in the art can treat a subject, such as a human patient, having fibrosis (e.g., pulmonary fibrosis, myelofibrosis, or fibrosis associated with chronic kidney disease) so as to reduce the symptoms of fibrosis or slow or stop the progression of fibrosis. The method of treatment can include diagnosing or identifying a subject as a candidate for treatment based on clinical tests for fibrosis (e.g., imaging tests, such as X-ray or CT scan). To treat the subject, a physician of skill in the art can administer to the subject a composition containing a polypeptide of SEQ ID NO: 1. The composition containing the polypeptide may be administered to the subject, for example, by subcutaneous injection to treat fibrosis, or can be locally administered (e.g., injected) to the fibrotic tissue or organ. The polypeptide of SEQ ID NO: 1 is administered in an amount of from 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) once every 28 days. The polypeptide of SEQ ID NO: 1 is administered in an amount sufficient to reduce the symptoms of fibrosis or slow or stop the progression of fibrosis.

Following administration of the composition to a patient, a practitioner of skill in the art can monitor the patient's improvement in response to the therapy by a variety of methods. For example, a physician can monitor the patient's fibrosis by performing imaging tests and can monitor the patient's symptoms using standard clinical tests. A finding that the patient's symptoms are reduced or that progression of the patient's fibrosis slows or stops following administration of the composition compared to test results prior to administration of the composition indicates that the patient is responding favorably to the treatment. Subsequent doses can be determined and administered as needed.

### Example 5

### Treatment of Pulmonary Hypertension by Administration of a Polypeptide of SEQ ID NO: 1

According to the methods disclosed herein, a physician of skill in the art can treat a subject, such as a human patient, having pulmonary hypertension (PH, e.g., PAH) so as to reduce the symptoms of PH or slow or stop the progression of PH. The method of treatment can include diagnosing or identifying a subject as a candidate for treatment based on standard clinical tests for PH (e.g., echocardiogram, electrocardiogram, chest X-ray, or right heart catheterization). To treat the subject, a physician of skill in the art can administer to the subject a composition containing a polypeptide of SEQ ID NO: 1. The composition containing the polypeptide may be administered to the subject, for example, by subcutaneous injection to treat PH. The polypeptide of SEQ ID NO: 1 is administered in an amount of from 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) once every 28 days. The polypeptide of SEQ ID NO: 1 is administered in an amount sufficient to reduce the symptoms of PH or slow or stop the progression of PH.

Following administration of the composition to a patient, a practitioner of skill in the art can monitor the patient's improvement in response to the therapy by a variety of methods. For example, a physician can monitor the patient's symptoms using standard clinical tests and patient self-reporting. A finding that the patient's symptoms are reduced the symptoms of PH or that progression of the patient's PH slows or stops following administration of the composition compared to test results prior to administration of the composition indicates that the patient is responding favorably to the treatment. Subsequent doses can be determined and administered as needed.

### Example 6

### Treatment of Metabolic Disease by Administration of a Polypeptide of SEQ ID NO: 1

According to the methods disclosed herein, a physician of skill in the art can treat a subject, such as a human patient, having a metabolic disease (e.g., obesity) so as to reduce body weight, body fat or percent body fat, or improve the serum lipid profile of the subject. To treat the subject, a physician of skill in the art can administer to the subject a composition containing a polypeptide of SEQ ID NO: 1. The composition containing the polypeptide may be administered to the subject, for example, by subcutaneous injection to treat obesity. The polypeptide of SEQ ID NO: 1 is administered in an amount of from 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) once every 28 days. The polypeptide of SEQ ID NO: 1 is administered in an amount sufficient to reduce body weight, body fat or percent body fat, or improve the serum lipid profile of the subject. Following administration of the composition to a patient, a practitioner of skill in the art can monitor the patient's improvement in response to the therapy by a variety of methods. For example, a physician can monitor the patient's symptoms using standard clinical tests and patient self-reporting. A finding that the patient's body weight, body fat, or percent body fat is reduced, or that the patient's serum lipid profile is improved following administration of the composition compared to test results prior to administration of the composition indicates that the patient is responding favorably to the treatment. Subsequent doses can be determined and administered as needed.

### Example 7

### Treatment of Thrombocytopenia by Administration of a Polypeptide of SEQ ID NO: 1

According to the methods disclosed herein, a physician of skill in the art can treat a subject, such as a human patient, having thrombocytopenia (e.g., thrombocytopenia associated with a myelodysplastic syndrome or myelofibrosis) so as to increase platelet levels (e.g., increase platelet count), increase platelet production, and/or increase megakaryocyte differentiation and/or maturation. To treat the subject, a physician of skill in the art can administer to the subject a composition containing a polypeptide of SEQ ID NO: 1. The composition containing the polypeptide may be administered to the subject, for example, by subcutaneous injection to treat thrombocytopenia. The polypeptide of SEQ ID NO: 1 is administered in an amount of from 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) once every 28 days. The polypeptide of SEQ ID NO: 1 is administered in an amount sufficient to increase platelet levels (e.g., increase platelet count), increase platelet production, and/or increase megakaryocyte differentiation and/or maturation.

Following administration of the composition to a patient, a practitioner of skill in the art can monitor the patient's improvement in response to the therapy by a variety of methods. For example, a physician can monitor the patient's platelet count using a blood test. A finding that the patient's platelet levels are increased (e.g., a finding of an increased platelet count) following administration of the composition compared to test results prior to administration of the composition indicates that the patient is responding favorably to the treatment. Subsequent doses can be determined and administered as needed.

### Example 8

### Treatment of Neutropenia by Administration of a Polypeptide of SEQ ID NO: 1

According to the methods disclosed herein, a physician of skill in the art can treat a subject, such as a human patient, having neutropenia (e.g., neutropenia associated with a myelodysplastic syndrome or myelofibrosis) so as to increase neutrophil levels (e.g., increase neutrophil count), increase neutrophil production, and/or increase the differentiation and/or maturation of progenitor cells (e.g., myeloid progenitors, myeloblasts, or myelocytes) into neutrophils. To treat the subject, a physician of skill in the art can administer to the subject a composition containing a polypeptide of SEQ ID NO: 1. The composition containing the polypeptide may be administered to the subject, for example, by subcutaneous injection to treat neutropenia. The polypeptide of SEQ ID NO: 1 is administered in an amount of from 1.5 mg/kg to 4.5 mg/kg (e.g., 1.5, 1.75, 2, 2.25, 2.5, 2.75, 3, 3.25, 3.5, 3.75, 4, 4.25, or 4.5mg/kg) once every 28 days. The polypeptide of SEQ ID NO: 1 is administered in an amount sufficient to increase neutrophil levels (e.g., increase neutrophil count), increase neutrophil production, and/or increase the differentiation and/or maturation of progenitor cells (e.g., myeloid progenitors, myeloblasts, or myelocytes) into neutrophils.

Following administration of the composition to a patient, a practitioner of skill in the art can monitor the patient's improvement in response to the therapy by a variety of methods. For example, a physician can monitor the patient's neutrophil count using a blood test. A finding that the patient's neutrophil levels are increased (e.g., a finding of an increased neutrophil count) following administration of the composition compared to test results prior to administration of the composition indicates that the patient is responding favorably to the treatment. Subsequent doses can be determined and administered as needed.

### Example 9

### Treatment of End-Stage Renal Disease by Administration of a Polypeptide of SEQ ID NO: 1

According to the methods disclosed herein, a physician of skill in the art can treat a subject, such as a human patient, having end-stage renal disease so as to increase EPO levels. To treat the subject, a physician of skill in the art can administer to the subject a composition containing a polypeptide of SEQ ID NO: 1. The composition containing the polypeptide may be administered to the subject, for example, by subcutaneous injection to treat end-stage renal disease. The polypeptide of SEQ ID NO: 1 is administered in an amount of from 1.5 mg/kg to 4.5 mg/kg (e.g., 1.5, 1.75, 2, 2.25, 2.5, 2.75, 3, 3.25, 3.5, 3.75, 4, 4.25, or 4.5 mg/kg) once every 28 days. The polypeptide of SEQ ID NO: 1 is administered in an amount sufficient to increase EPO levels, increase EPO receptor levels, and/or slow progression of the disease.

Following administration of the composition to a patient, a practitioner of skill in the art can monitor the patient's improvement in response to the therapy by a variety of methods. For example, a physician can monitor the patient's EPO levels using a blood test and can measure kidney function using blood tests, urine tests, and imaging tests. A finding that the patient's EPO levels are increased or that the disease is progressing more slowly following administration of the composition compared to test results prior to administration of the composition indicates that the patient is responding favorably to the treatment. Subsequent doses can be determined and administered as needed.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent or patent application was specifically and individually indicated to be incorporated by reference in its entirety.

Other embodiments are within the following claims.

1. A method of treating a human subject having a bone disease or pulmonary hypertension (PH), the method comprising the step of administering to the subject a polypeptide of SEQ ID NO: 1 in an amount of 1.5 mg/kg to 4.5 mg/kg at a frequency of once every 28 days.

2. The method of claim 1, wherein the subject has a bone disease.

3. The method of claim 1 or 2, wherein the bone disease is osteoporosis, osteopenia, osteopetrosis, bone fracture, bone cancer or cancer metastasis-related bone loss, Paget's disease, renal osteodystrophy, treatment-related bone loss, osteogenesis imperfecta, neuromuscular disease-related bone loss, burn-induced bone loss, anorexia-related bone loss, diet-related bone loss, bone loss associated with the treatment of obesity, low gravity-related bone loss, or immobility-related bone loss.

4. The method of claim 3, wherein the bone disease is osteoporosis.

5. The method of claim 3 or 4, wherein the osteoporosis is primary osteoporosis.

6. The method of claim 3 or 4, wherein the osteoporosis is secondary osteoporosis.

7. The method of claim 3, wherein the bone disease is osteogenesis imperfecta.

8. The method of claim 1, wherein the subject has PH.

9. The method of claim 1 or claim 8, wherein the PH is pulmonary arterial hypertension (PAH), venous PH, hypoxic PH, thromboembolic PH, or miscellaneous PH.

10. The method of claim 9, wherein the PH is PAH.

11. The method of claim 9 or 10, wherein the PAH is idiopathic PAH, heritable PAH, or PAH associated with HIV infection, schistosomiasis, cirrhosis of the liver, a congenital heart abnormality, portal hypertension, pulmonary veno-occlusive disease, pulmonary capillary hemangiomatosis, a connective tissue disorder, an autoimmune disorder, drug use or abuse, a toxin, or congenital systemic-pulmonary intracardiac shunt.

12. The method of claim 9 or 10, wherein the method further comprises administering a PAH background therapy.

13. The method of claim 9 or 10, wherein the human subject is on a PAH background therapy.

14. A method of treating a human subject having fibrosis, a disease or condition involving muscle weakness or atrophy, a metabolic disease, thrombocytopenia, neutropenia, or a disease or condition that can be treated with erythropoietin or an erythropoiesis-stimulating agent, the method comprising the step of administering to the subject a polypeptide of SEQ ID NO: 1 in an amount of 1.5 mg/kg to 4.5 mg/kg once every 28 days.

15. The method of claim 14, wherein the subject has fibrosis.

16. The method of claim 14 or 15, wherein the fibrosis is chemotherapeutic drug-induced fibrosis, radiation-induced fibrosis, pulmonary fibrosis, hepatic fibrosis, renal fibrosis, corneal fibrosis, heart fibrosis, bone marrow fibrosis, myelofibrosis, mediastinal fibrosis, retroperitoneal fibrosis, osteoarticular fibrosis, arthrofibrosis, tissue fibrosis, a tumor stroma, a desmoplastic tumor, a surgical adhesion, a hypertrophic scar, or a keloid, or is fibrosis associated with a wound, a burn, hepatitis B or C infection, fatty liver disease, Schistosoma infection, kidney disease, chronic kidney disease, heart disease, macular degeneration, retinal or vitreal retinopathy, Crohn's disease, systemic or local scleroderma, atherosclerosis, or restenosis.

17. The method of claim 14, wherein the subject has a disease or condition involving muscle weakness or atrophy.

18. The method of claim 14 or 17, wherein the disease or condition involving muscle weakness or atrophy is a neuromuscular disease, sarcopenia, cachexia, disuse atrophy, treatment-related muscle loss or atrophy, hypotonia, muscle loss or atrophy associated with hypoxia, or muscle loss or atrophy associated with a burn injury.

19. The method of claim 14, wherein the subject has a metabolic disease.

20. The method of claim 14 or 19, wherein the metabolic disease is obesity, Type 1 diabetes, or Type 2 diabetes.

21. The method of claim 14, wherein the subject has thrombocytopenia.

22. The method of claim 14 or 21, wherein the thrombocytopenia is familial thrombocytopenia, immune thrombocytopenia, or is associated with a bone marrow defect, a myelodysplastic syndrome, bone marrow transplantation, myelofibrosis, myelofibrosis treatment, ineffective hematopoiesis, Gaucher disease, aplastic anemia, Fanconi anemia, Diamond Blackfan anemia, Shwachman Diamond syndrome, heavy alcohol consumption, cirrhosis of the liver, cancer, an autoimmune disease, a viral infection, a bacterial infection, an enlarged spleen, a vitamin deficiency, cancer treatment, thrombotic thrombocytopenia purpura, idiopathic thrombocytopenia purpura, disseminated intravascular coagulation, hemolytic uremic syndrome, paroxysmal nocturnal hemoglobinuria, a reduction of platelets caused by medication, a dilution of platelets caused by a blood transfusion, hematopoietic stem cell transplantation, acquired amegakaryocytic thrombocytopenia, Pearson syndrome, dyskeratosis congenita, or contraindication to transfusion.

23. The method of claim 14, wherein the subject has neutropenia.

24. The method of claim 14 or 23, wherein the neutropenia is familial neutropenia, chronic idiopathic neutropenia, or is associated with a bone marrow defect, a myelodysplastic syndrome, bone marrow transplantation, myelofibrosis, ineffective hematopoiesis, aplastic anemia, Fanconi anemia, Diamond Blackfan anemia, Shwachman Diamond syndrome, paroxysmal nocturnal hemoglobinuria, Pearson syndrome, dyskeratosis congenita, cancer, a vitamin deficiency, an enlarged spleen, an autoimmune disease, a viral infection, a bacterial infection, cancer treatment, a reduction in neutrophils caused by medication, inflammation, hematopoietic stem cell transplantation, or contraindication to transfusion.

25. The method of claim 14, wherein the subject has a disease or condition that can be treated with erythropoietin or an erythropoiesis-stimulating agent.

26. The method of claim 14 or 25, wherein the subject has end-stage renal disease, renal insufficiency, polycythemia, hemochromatosis, a disease or condition associated with dysfunction of endothelial progenitor cells, a disease or condition having an autoimmune or inflammatory component, a neurological disorder or inflammatory brain disease, gastrointestinal dysmotility, a disease of the endocrine system, a disease of the reproductive system, aging, pregnancy, a menstrual disorder, ischemia or an ischemic disorder or condition, hypoxia or a hypoxic disorder or condition, an ulcer, a burn, a wound, ischemia-reperfusion injury, asthma, hypertension, a viral disease or infection, a systemic microbial infection, a gastrointestinal disease, arterial sclerosis, cancer, psychosis, a genetic disease, an inflammatory disease, graft-versus-host disease, cardiovascular disease, an allergy, or arthritis.

27. The method of any one of claims 1-26, wherein the polypeptide of SEQ ID NO: 1 is administered in an amount of 1.5 mg/kg to 2.5 mg/kg.

28. The method of claim 27, wherein the polypeptide of SEQ ID NO: 1 is administered in an amount of 1.5 mg/kg.

29. The method of any one of claims 1-26, wherein the polypeptide of SEQ ID NO: 1 is administered in an amount of 2.5 mg/kg to 3.5 mg/kg.

30. The method of claim 29, wherein the polypeptide of SEQ ID NO: 1 is administered in an amount of 3 mg/kg.

31. The method of any one of claims 1-26, wherein the polypeptide of SEQ ID NO: 1 is administered in an amount of 3.5 mg/kg to 4.5 mg/kg.

32. The method of claim 31, wherein the polypeptide of SEQ ID NO: 1 is administered in an amount of 4.5 mg/kg.

33. The method of any one of claims 1-32, wherein the polypeptide of SEQ ID NO: 1 is administered subcutaneously.

34. The method of any one of claims 1-33, wherein the polypeptide of SEQ ID NO: 1 is administered as a homodimer.
